(19) United States
(12) Patent Application Publication
(10) Pub. No.: US 2022/0370777 A1
(43) Pub. Date: Nov. 24, 2022

(54) ACTIVE MICRONEEDLES FOR ENHANCED PAYLOAD UPTAKE

(71) Applicants: Joseph WANG, San Diego, CA (US); Nicole STEINMETZ, San Diego, CA (US); Miguel Angel LOPEZ RAMIREZ, La Jolla, CA (US); Fernando SOTO, La Jolla, CA (US); Nisarg SHAH, La Jolla, CA (US); The Regents of the University of California, Oakland, CA (US)

(72) Inventors: Joseph Wang, San Diego, CA (US); Nicole Steinmetz, San Diego, CA (US); Miguel Angel Lopez Ramirez, La Jolla, CA (US); Fernando Soto, La Jolla, CA (US); Nisarg Shah, La Jolla, CA (US)

(21) Appl. No.: 17/597,990

(22) PCT Filed: Jul. 31, 2020
(86) PCT No.: PCT/US2020/044660

§ 371 Date: Jan. 31, 2022

Related U.S. Application Data
(60) Provisional application No. 62/881,790, filed on Aug. 1, 2019.

(51) Int. Cl. A61M 37/00; A61K 47/32; A61K 47/38

(52) U.S. Cl. CPC A61M 37/0015; A61K 47/32; A61K 47/38; A61M 2037/0046

(57) ABSTRACT

Disclosed are devices, systems and methods for in situ payload delivery via a degradable microneedle. In some aspects, a microneedle therapeutic payload delivery device includes a substrate; an activation particle; and one or more degradable microneedle structures coupled to the substrate and including a polymeric matrix structured to embed the activation microparticle and one or more therapeutic payloads, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of a biofluid surrounding the microneedle structure to dissolve and allow the one or more therapeutic payloads and the activation particle to the surrounding biofluid.

### CROSS-REFERENCE TO RELATED APPLICATIONS

This patent document claims priorities to and benefits of U.S. Provisional Patent Application No. 62/881,790 entitled “ACTIVE MICRONEEDLES FOR ENHANCED PAYLOAD UPTAKE” filed on Aug. 1, 2019. The entire content of the aforementioned patent application is incorporated by reference as part of the disclosure of this patent document.

### TECHNICAL FIELD

This patent document relates to devices, systems and methods for in situ delivery of cargo payload to a biological organism or tissue, for example, via a degradable microneedle.

### BACKGROUND

Multiple methods have been proposed for the delivery of a wide variety of payloads for transdermal delivery, including diffusion transport, encapsulation in nano-microparticles, as well as enhanced delivery using external fields such as temperature.

### SUMMARY

The use of microneedles has facilitated the painless and localized delivery of drugs across the skin. Although their use has successfully addressed the treatment of multiple diseases, the efficacy of these devices has been limited by the slow diffusion of drug through the skin or the requirement of external triggers to achieve a faster active delivery. For example, although microneedles have been a very successful platform for the delivery of these active compounds, the main disadvantages of conventional approaches are: (1) a two-step administration process is typically needed; (2) not having morphology such as an insufficiently sharp tip (suffering decreased penetration ability); and (3) requiring an efficient coating procedure. Common diffusion technology incorporated into microneedle patches provide a well-established platform and commodity to users. However, diffusion alone presents a linear release kinetic behavior and may not be sufficient to overcome the challenges of barriers transdermal technology, such as dense local tissue.

A key challenge of transdermal technology is the fabrication of a robust delivery platform that can work autonomously, that presents long-lasting operation and that is fully biocompatible. Microneedles according to the currently disclosed technology can enhance the release kinetics of antibody and any desired payload compared to actual methods and can easily pierce the skin due to its sharp tip. Furthermore, this currently disclosed technology offers multiple flexibilities regarding operation, dosage, storage, particle tuning size and release enhancing percentage.

Disclosed are devices, systems and methods for in-situ delivery of a cargo payload into tissues, human or non-human (e.g., animals, plants), via microneedles, including a degradable microneedle loaded with the payload that controllably releases the payload when inserted within the tissue. The microneedles according to some embodiments of the disclosed technology are loaded with the targeted payload along with active particles, which can serve as an active pump, and can enhance payload uptake and delivery in the organism.

Various examples of embodiments and implementations are disclosed. For example, in some implementations, the particle localized convective fluid transport of the presently disclosed technology remains active for more than 20 min, and the stability of these without application can last for long periods of time. The disclosed technology presents vast flexibility in terms of the size of the particle, pH environment and the concentration of the payload, is not limited to specific dimensions (diameter and height), a specific material (polymer), or active particles, and lastly, has been demonstrated to work in the delivery of therapeutic agents, antibodies, proteins, genetic material, particles, viruses, virus-like particles as well as in small or big therapeutic molecules.

Such active microneedle delivery can offer a faster and greater distribution of the target payload into the target tissue as compared to conventional delivery techniques. The ease of use of the disclosed transdermal technology can provide a fast, autonomous, fully biocompatible, and reproducible way of delivering payloads into deep tissue, and without the need of externally triggered equipment, e.g., as compared to diffusion-based methods and hypodermic standard needles. The disclosed technology provides a good alternative for treatment delivery that is not associated with pain, injury and, in some cases, extreme fear in patients. It has great potential in clinical translation due to its fully biocompatible and autonomous nature.

In some aspects, an autonomous, degradable and active microneedle delivery platform employs magnesium (Mg) microparticles loaded within the microneedle patch, as the built-in engine for deeper and faster intradermal payload delivery. The active microneedle patch uses the bodily fluids to activate these Mg particles, leading to an enhanced transdermal delivery of the loaded payload which could result in shorter times and better distribution when compared to passive microneedles. The embedded Mg particles can substantially increase the displacement of tracer microparticles through localized fluid convection resulting from their microbubbles production.

Example data and results of various implementations are described herein. For example, the drug release kinetics of example implementations of various embodiments were tested in vitro by measuring the amount of IgG antibody (Ab) (model drug) that passed through phantom tissue and pigskin barriers. Moreover, the active microneedle delivery mechanism was shown to induce an immune response in a syngeneic B16F10 mouse melanoma model with significantly longer life expectancy when compared to immune therapy anti-CTLA-4 or corresponding use of passive microneedles.

In some embodiments, a microneedle patch is configured for combinatorial delivery using spatially-resolved active and passive microneedle zones, toward fast and deep delivery along with slow sustained release, respectively. Such versatile and effective autonomous dynamic microneedle delivery technology offers considerable promise for a wide range of biomedical and personal applications in connection to improved delivery of different drugs, vaccines, cosmetics and gene therapy modalities, towards greatly improved outcome, convenience and costs.

In some embodiments, a microneedle patch is configured for virus immunotherapy delivery and distribution into a tumor tissue more effectively, therefore targeting the tumor microenvironment more broadly as compared to soluble bolus administration. The combination of active delivery and enhanced distribution greatly enhances efficacy of a plant virus cancer immunotherapy applied as in situ vaccine.

In some aspects, a microneedle therapeutic payload delivery device includes a substrate; an activation particle; and one or more degradable microneedle structures coupled to the substrate and including a polymeric matrix structured to embed the activation microparticle and one or more therapeutic payloads, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of a biofluid surrounding the microneedle structure to dissolve and allow the one or more therapeutic payloads and the activation particle to the surrounding biofluid.

In some aspects, a method for autonomously delivering a payload into a biofluid via microneedles includes providing a microneedle patch device that includes a substrate and one or more degradable microneedle structures coupled to the substrate and including a polymeric matrix structured to embed a activation microparticle and one or more payload substances, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of the biofluid that would surround the microneedle structure; applying the microneedle patch device to skin of a subject such that the one or more degradable microneedle structures are inserted into a tissue; dissolving the one or more degradable microneedles in the fluid based on exposure of the one or more degradable microneedles to the environmental parameter; and reacting the activation particle to the biofluid to cause the one or more payload substances to disperse away from the one or more degradable microneedle structures, thereby enhancing penetration of the one or more payload substances into the tissue.

The subject matter described in this patent document can be implemented in specific ways that provide one or more of the following features.

### BRIEF DESCRIPTION OF THE DRAWINGS

FIG. 1A shows a block diagram depicting an example embodiment of a microneedle delivery platform device in accordance with the present technology.

FIG. 1B shows a schematic of a microneedle structure and diffusion of the therapeutic agent within a microneedle structure in accordance with example embodiments disclosed herein.

FIGS. 1C and 1D show a data plot and diagram depicting theoretical loading of an example embodiment of a microneedle patch in accordance with the present technology.

FIGS. 2A-2H show diagrams and images depicting an example embodiment and implementations of a degradable and autonomous active microneedle delivery platform based on a robust built-in micropump system for enhanced payload permeation in accordance with the present technology.

FIGS. 3A-3H show active particle microneedle performance, activation, degradation and mechanical testing in accordance with example embodiments disclosed herein.

FIGS. 4A-4C show time-lapse images of the dissolution rate, fluid mixing, and permeation of antibodies embedded within different microneedles in accordance with example embodiments disclosed herein.

FIGS. 5A-5H show active fluid transport (localized streaming) based on Mg particles embedded in dissolvable microneedles in accordance with example embodiments disclosed herein.

FIG. 6A is a schematic fabrication process for preparing microneedles comprising Mg particles with an enteric coating in accordance with example embodiments disclosed herein.

FIGS. 6B-6C show scanning electron micrograph (SEM) images and dissolution rates microneedles having an enteric coating over Mg particles of FIG. 6A, respectively in accordance with example embodiments disclosed herein.

FIGS. 7A-7C show a schematic of an active microneedle, a flow trace of the Mg particles, and time lapse images of the active microneedles, respectively in accordance with example embodiments disclosed herein.

FIG. 8 are images of COMSOL multiphysics simulation of the flow generated without and with particles within the microneedle structure in accordance with example embodiments disclosed herein.

FIGS. 9A-9L show an example evaluation of the in vitro payload release performance of common passive and active microneedles by fluorescence, electrochemical and spectrophotometric techniques in accordance with example embodiments disclosed herein.

FIGS. 10A-10D show evaluation of ex vivo dye release performance of passive and active microneedles in accordance with example embodiments disclosed herein.

FIGS. 11A-11D show in vivo skin cancer treatment using anti-CTLA-4 antibodies delivered by active vs. passive microneedles and intratumoral injection in B16F10 dermal melanoma model in accordance with example embodiments disclosed herein.

FIGS. 12A-12I show an example combinatorial drug microneedle patch for the simultaneous dual delivery of different payload and methods of fabricating the same in accordance with example embodiments disclosed herein.

FIGS. 13A-13G show a formulation of a dissolvable active microneedle (MN) patch and corresponding characterization for the delivery of plant virus nanoparticles (cowpea mosaic virus, CPMV) in accordance with example embodiments disclosed herein.

FIGS. 14A-14F show CPMV in situ vaccination administered by active microneedle (MN) patches, passive MN patches or intratumoral injection in B16F10 dermal melanoma model in accordance with example embodiments disclosed herein.

FIGS. 15A-15F show tumor growth suppression and survival with CPMV in situ vaccination administered by active MN, passive MN, and intratumoral injection in B16F10 dermal melanoma model in accordance with example embodiments disclosed herein.

FIGS. 16A-16D show in vivo and ex vivo imaging of Cy5-conjugated CPMV (Cy5-CPMV) in situ vaccination of B16F10 melanomas administered by active MN, passive MN, and intratumoral injection in accordance with example embodiments disclosed herein.

FIGS. 17A-17B show percentage of total cells analyzed that were CD45+ at 4 hours (left) and 24 hours (right) after treatment with passive MN or active MN for C57BL/6 mice bearing dermal B16F10 tumors in accordance with example embodiments disclosed herein.

FIGS. 18A-18J show innate immune tumor infiltrate profiles and percentages of intratumoral CD45+ cells after treatment passive MN or active MN for C57BL/6 mice bearing dermal B16F10 tumors in accordance with example embodiments disclosed herein.

FIGS. 19A-19D show Cy5-CPMV loaded active and passive MN patch characterization in accordance with example embodiments disclosed herein.

FIGS. 20A-20B show systemic anti-tumor immune response following CPMV microneedle administration in accordance with example embodiments disclosed herein.

FIGS. 21A-21D show intratumoral innate immune cell profile following CPMV microneedle administration in accordance with example embodiments disclosed herein.

FIG. 22 shows images of a gating strategy for detection of CD44^{hi}IFN-γ+CD8+ T cells in splenocyte interferon gamma release assay in accordance with example embodiments disclosed herein.

### DETAILED DESCRIPTION

Current efforts and innovations on drug delivery platforms have the potential to enhance therapeutic efficiency. Existing therapy modalities (e.g., pill- or liquid-based oral delivery, needle-injections, suppository delivery, etc.) have successfully addressed basic medicine delivery requirements. Yet, there are urgent needs to develop local delivery platforms that can address and overcome the pain and fear from hypodermic injections and poor absorption and toxicity-related problems associated with the systemic delivery by pills-all while maintaining cost-efficacy and ease of use.

One plausible solution relies on the use of microneedles towards painless and localized delivery of drugs across the skin. For example, microneedles have proved useful for enhancing the drug permeability through the epidermis. Furthermore, this route offers autonomy and ease of use, as the therapeutic payload remains released over prolonged periods based on the material properties or by the inclusion of encapsulated smart drug-loaded particles.

Existing microneedle drug-delivery platforms usually rely on passive diffusion, which limits the penetration depth and distribution of the therapeutic payloads. While a gradual release of equivalent doses of therapeutics from transdermal microneedles can improve the efficacy compared to conventional treatment methods, diverse clinical circumstances may benefit from a rapid, burst payload release. For example, passive diffusion of drugs from the microneedles may still exhibit limited permeation within tumor microenvironments, where tissue penetration based solely on diffusion-based methods may also be more restricted. In this direction, diverse external stimuli have been employed to enhance the drug permeation through the epidermis; these external triggers include electroporation, ultrasound, light, and temperature, Yet, the requirement of external (often costly and bulky) equipment limits the widespread use of these stimuli to specialized centralized lab settings but not to field/remote locations. Thus, it is desirable to provide advantages of both autonomous and active delivery into a single platform, towards coupling of the benefits of autonomous delivery and reducing the time necessary for achieving high therapeutic efficiency.

Disclosed are devices, systems and methods for in-situ, active delivery of a payload into tissues via degradable microneedles. The disclosed devices, systems and methods utilize an environmental parameter of the biofluid to trigger degradation of one or more materials of the microneedles that encapsulate both a payload and an activation particle (e.g., microparticle or nanoparticle) to react in the biofluid and autonomously facilitate delivery of the payload to the surrounding biological environment. The disclosed devices, systems and method stand in contrast to convention microneedle techniques, which suffer from the limited diffusion (e.g., penetration depth) and inefficient distribution of the payload; and thus, require costly and bulky equipment.

In some aspects, disclosed is a degradable and autonomous active microneedle delivery platform based on a robust built-in micropump system, for enhanced payload permeation. For example, in some embodiments, a microneedle therapeutic payload delivery device includes a substrate; an activation particle; and one or more degradable microneedle structures coupled to the substrate and including a polymeric matrix structured to embed the activation microparticle and a therapeutic payload, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of a biofluid surrounding the microneedle structure to dissolve and allow the therapeutic payload and the activation microparticle to the surrounding biofluid.

In some example embodiments, the microneedle patch includes a degradable polymeric microneedle body loaded with a therapeutic payload and active motor-Mg microparticles. In implementations, for example, once the microneedle patch is attached to a patient's skin and the polymeric microneedles are exposed to subcutaneous biofluid (e.g., interstitial fluid (ISF)), the polymeric microneedles are able to dissolve such that the embedded Mg particles react (e.g., instantaneously) with the surrounding the subcutaneous biofluid, resulting in a rapid generation of hydrogen bubbles, that induces distinct vortex flow fields, and lead to a powerful pumping-like action, and dynamic transport of the embedded therapeutic payload.

Several example embodiments and implementations of the disclosed degradable microneedle payload-delivery technology are described below. For example, as discussed in further detail later in this patent document, implementations of an example embodiment of the degradable microneedle patch examined the drug release kinetics (in an example model) to test in vitro the degradable and autonomous active microneedle delivery platform, e.g., by measuring the amount of therapeutic payload that passed through phantom tissue and pigskin barriers, which presented enhanced permeation and distribution when compared to passive microneedles. Moreover, the advantages of the active platform's therapeutic efficacy in an animal melanoma model are discussed herein.

The versatility of the example approach is demonstrated by integrating spatially-resolved active and passive microneedles in the same patch, which can provide combinatorial drug delivery including active and passive segments. Such active microneedle delivery offers ease of use as an “all in one device,” providing an autonomous, biocompatible, and efficient alternative for faster release kinetics of payloads through the skin while preventing the need of external activation (and related trigger equipment). Thus, it holds considerable promise to reduce the time necessary to achieve high therapeutic efficiency of different drugs, vaccines, and gene therapy modalities towards diverse biomedical applications.

### Active Microneedle Enhanced Drug Delivery

In some aspects, the microneedle according to the presently disclosed technology can include a sharp array (solid or hollow) which serves to penetrate into the tissue. The microneedle material can be made from diverse transient degradable materials (e.g., polyvinylpyrrolidone (PVP), carboxymethylcellulose (CMC), hyaluronic acid (HA), sodium alginate (SA), Pullulan, starches and/or sugars), that start to dissolve upon contact with physiological and low pH medium (e.g., less than pH 7). The use of the optional systems to monitor the delivery can be integrated into the needle.

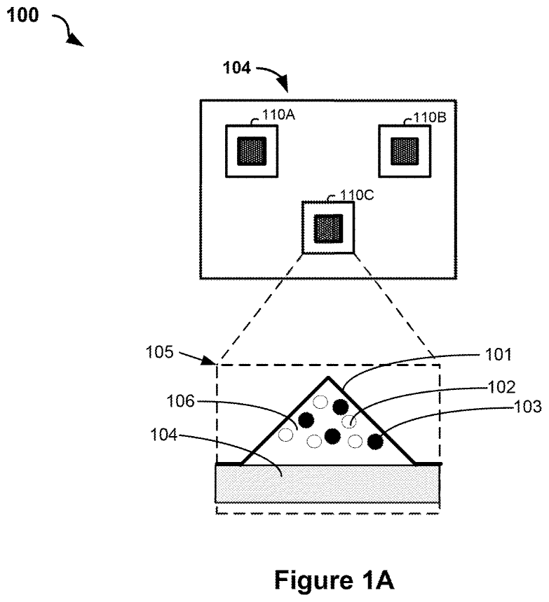

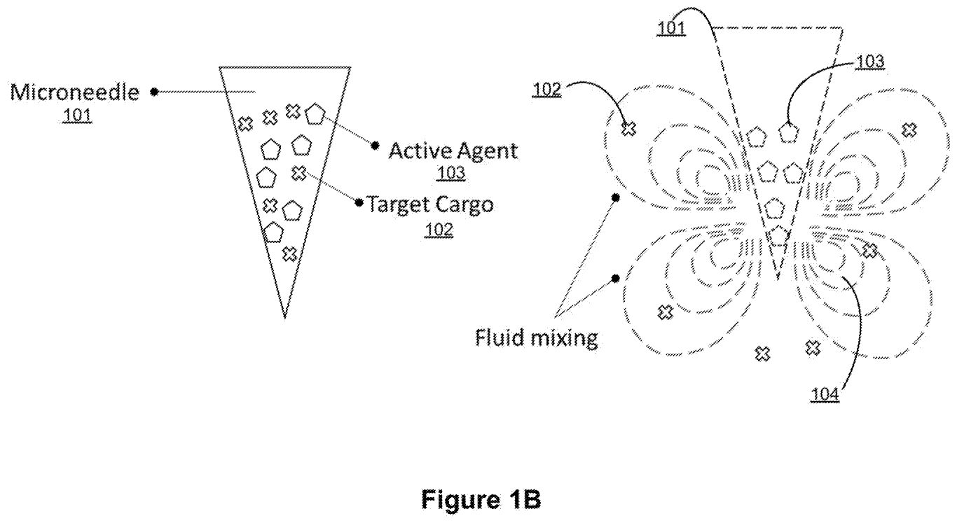

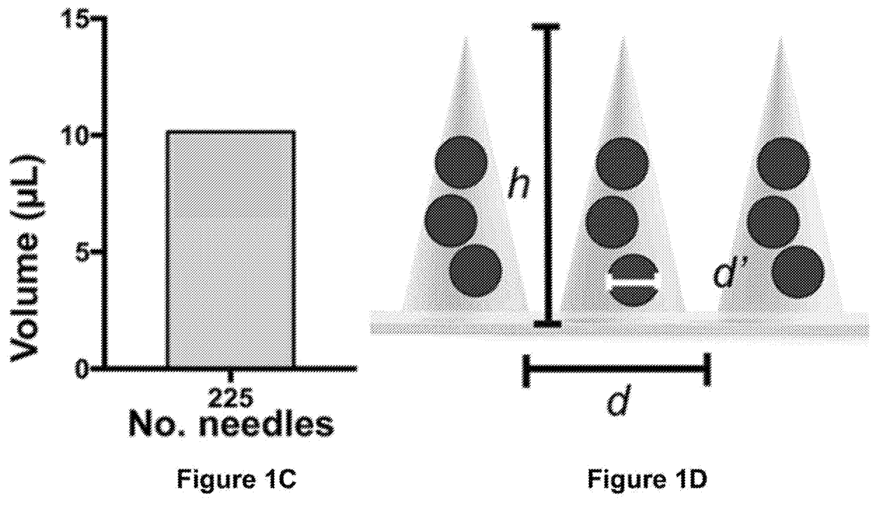

FIG. 1A shows a block diagram of an example embodiment of an active microneedle delivery system 100 in accordance with the present technology. The active microneedle delivery system 100 includes one or more microneedle structures 110 (e.g., shown as microneedle structures 110A, 110B, 110C in this example) disposed on a substrate 104. The active microneedle delivery system 100 is configured to embed a payload 102 (e.g., molecular payload, viral payload, etc.) within the one or more microneedle structures 110, which can include a degradable material such that the microneedle structure(s) 110 degrade (e.g., dissolve) in a biofluid upon insertion of the microneedle structure(s) 110. In some embodiments of the active microneedle delivery system 100, the one or more microneedle structures 110 include the payload 102 and an activation particle 103 to facilitate the delivery and/or uptake of the payload in the biofluid. The activation particle 103 is sometimes referred to as an “active particle.”

The block diagram of FIG. 1A includes an inset 105 showing a microneedle structure 101 (of the one or more microneedles structures 110) interfaced with the substrate 104 and depicting a plurality of the payload substance 102 and a plurality of the activation particle 103 embedded in a body 106 of the microneedle structure 101. For example, in some embodiments, the payload 102 and the activation particle 103 are embedded within the microneedle structure 101 by a polymeric matrix that constitutes the body 106 of the microneedle structure 101.

In various implementations of the active microneedle delivery system 100, the payload 102 is a therapeutic agent, which can be one or more of drugs, particles, molecules, genetic material, proteins, virus or viral vectors, and/or enzymes (or a combination thereof).

In various embodiments, the activation particle 103 can include a nanoparticle or a microparticle. For example, the activation particle 103 can be configured as a Mg microparticle that generates a propulsive force to drive the payload 102 into an affected area of the host to which the system 100 is deployed (e.g., a tumor).

In some embodiments of the active microneedle delivery system 100, for example, the substrate 104 includes an adhesive layer that adheres to the microneedle structure 101; and in some embodiments, the substrate 104 is a single adhesive material that binds to the base surfaces of the microneedle structure(s) 110. In some embodiments, the substrate 104 can include a medical adhesive (e.g., Tegaderm) and/or non-dissolving polymeric materials, e.g., polydimethylsiloxane (PDMS), polyvinyl alcohol (PVA), or other.

FIG. 1B shows a schematic diagram of the microneedle structure 101 including the payload 102 (e.g., target cargo) and the activation particle 103 (e.g., active agent) embedded therein (left schematic) and of the microneedle structure 101 undergoing degradation (right schematic) to autonomously and controllably release the payload 102, such that the activation particle 103 facilitates the release and uptake of the payload 102 by the surrounding environment (e.g., local cells, tissue or other biological entity of the surrounding biofluid). As shown in the schematic diagram, the microneedle structure 101 including the payload 102 and the active particle 103 is soluble upon contact with a fluid so as to allow the rapid dispersion of the payload 102 and active particles 103 into an affected area of the host. In some implementations, for example, the active particles 103 (e.g., Mg) are consumed by the interstitial fluid, resulting in the rapid production of H₂ bubbles, thus inducing distinct vortex flow fields 104 at the localized application site, which results in the enhancement of the permeation and diffusion of the loaded payload 102.

FIG. 1C shows a data plot depicting a theoretical loading profile of an example embodiment of the active microneedle delivery system 100, e.g., containing 225 conical microneedles in the array. As shown in the data plot, a peak value of payload packing (e.g., sphere payload particles) within each conical structure was determined to be ˜66.6%. Based on the volume of the example conical microneedle structures and payload, it was estimated that the microneedle tips can load a total volume of ˜10.13 μL in the tips.

FIG. 1D shows a diagram of the relative dimensions of the example conical microneedle structures estimated for payload packing in FIG. 1C. Calculations to determine the loading amount were based on the volume of the example conical microneedle structures (V=πr²h/3) and the volume of example spherical payload particles (V=4πr′³/3), where r is the radius (i.e., d=2r) of the conical microneedle structure, h is the height of the conical microneedle structure, and r′ is the radius of the payload (i.e., d′=2r′).

In some example implementations, the activation entity is an active microparticle comprised of Magnesium (Mg⁰) metal. Yet, notably, the disclosed microneedles can include but are not limited to the use of activation entities such as micro/nanoparticles (e.g., Mg⁰, calcium carbonate CaCo₃, zinc Zn⁰), chemically modified micro/nanoparticles (micro/nanomotors) with biocatalytic enzymes (e.g., urease, catalase, glucose oxidase), spherical (Janus) and rocket type micro/nanomotors made of inorganic materials (e.g., Mg⁰, Zn⁰, gold Au⁰, titanium dioxide TiO₂), or micro/nanoparticles modified metal organic frameworks (MOF) as catalytic engines and porous carriers (e.g., zeolitic imidazolate framework-67 and 8). These activation entities incorporated in microneedle patches include materials that enable the conversion of local chemical fuels (e.g., interstitial fluid, sweat), or external fields (e.g., magnetic, ultrasound, light) into leading forces (micropumps) within the application site, inducing fluid transport, enhancing and improving the distribution, penetration and efficacy of a therapeutic.

A wide variety of cargo payloads can be loaded and released, which include but are not limited to therapeutic agents, drugs, particles, molecules, genetic material, proteins, enzymes, etc., using singular or combination therapy. Diverse cargo therapeutics can be efficiently loaded within the microneedle embodiment, entrapped or coated in their free form or labeled/within micro/nanoparticles, which include but are not limited to: immune oncology agents, such as immune checkpoint inhibitors (e.g., anti-CTLA-4, anti-PD-1/PD-L1), chemotherapeutic agents (e.g., alkylating agents, mitotic inhibitors, antimetabolites, topoisomerase inhibitors), chronic pain medication (e.g., antidepressants, anticonvulsants, muscle relaxants, nonsteroidal anti-inflammatory drugs), cardiovascular medication (e.g., anticoagulants, ACE inhibitors, cholesterol-lowering agents, vasodilators), anti-aging agents (e.g., retinoids, ascorbic acid, hydroxy acids, antioxidants), antiviral therapeutics (e.g., non-retroviral antiviral agents, antiretroviral agents), vaccines (e.g., live attenuated, inactivated, recombinant, conjugated, toxoid), antibacterial drugs (e.g., inhibition of cell wall synthesis, inhibition of protein synthesis, inhibition of bacterial nucleic acid synthesis), micronutrients (e.g., vitamins, minerals) and gene editing effectors (e.g., CRISPR-Cas9, TALE nucleases), using singular or combinatorial microneedle patches for a fast or sustained delivery.

In some example implementations, the microneedles with active fluid transport can work as a pump towards enhancing mass transport and fluid mixing to increase the release kinetics and distribution of the payload, for example, to deliver insulin to a patient or towards oral delivery of anesthetic (e.g., like that illustrated in FIG. 1).

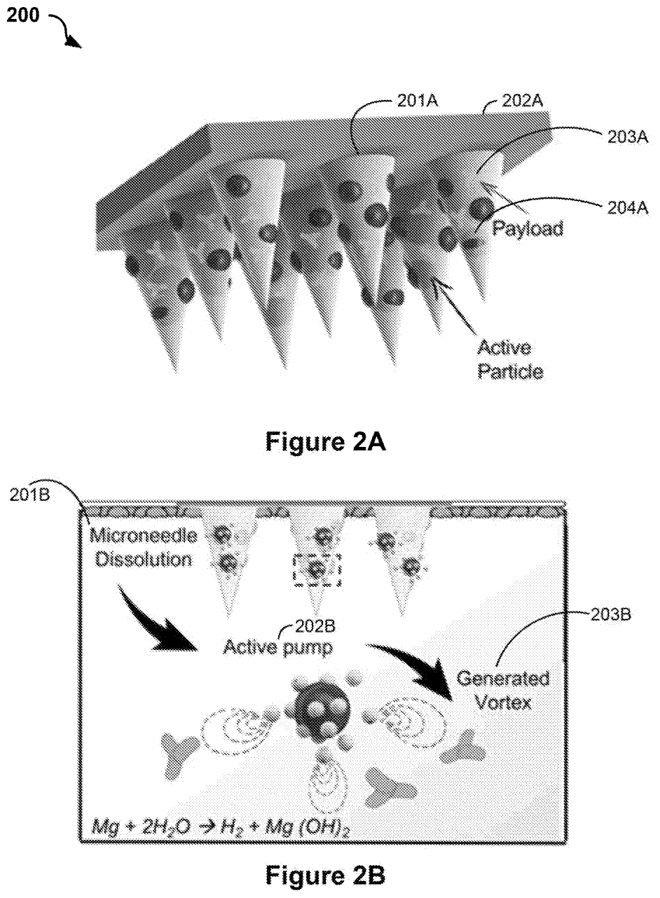

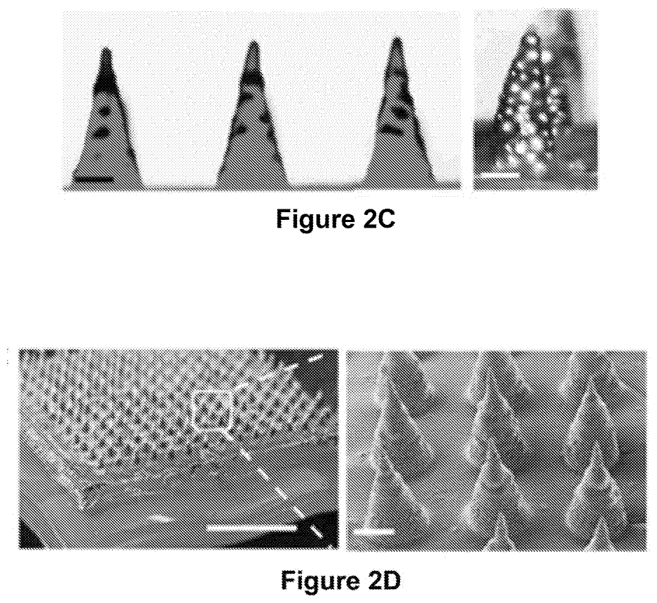

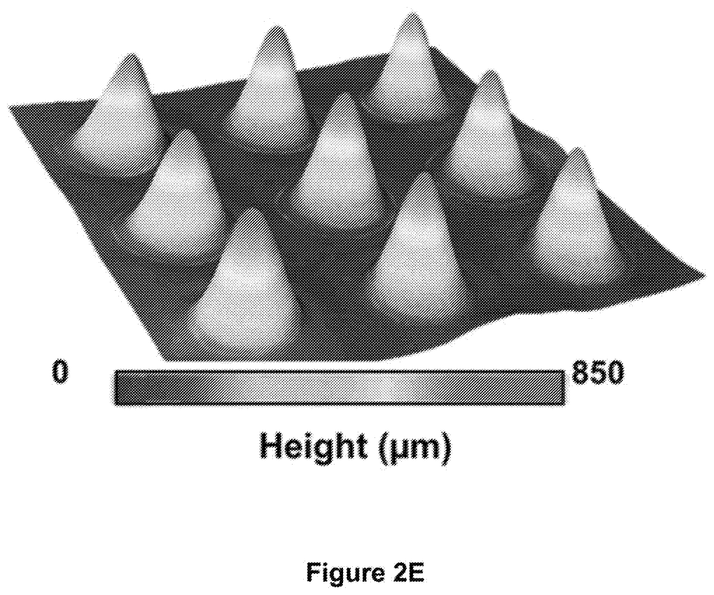

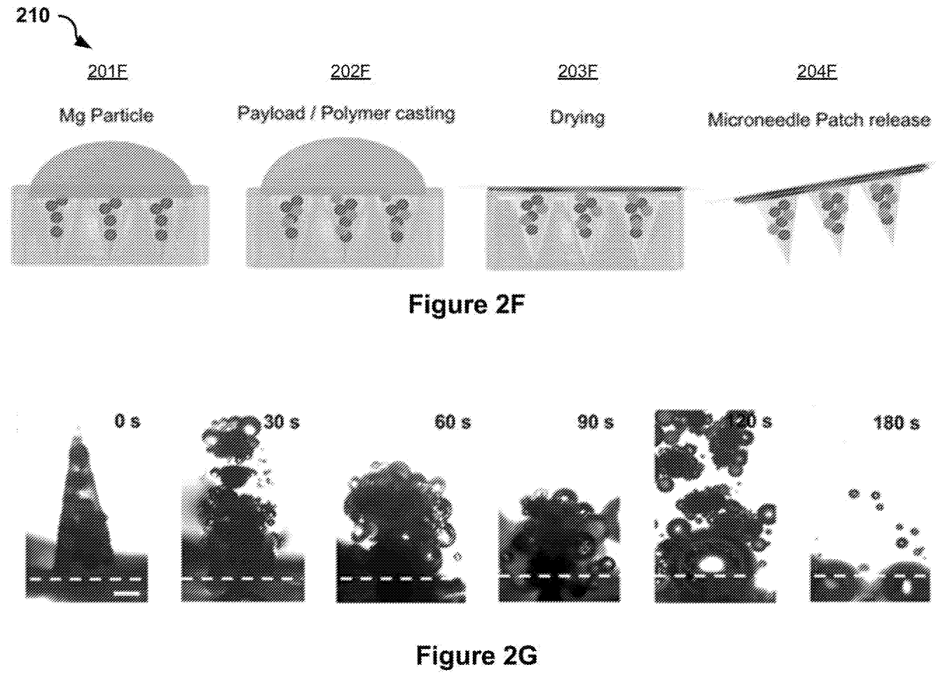

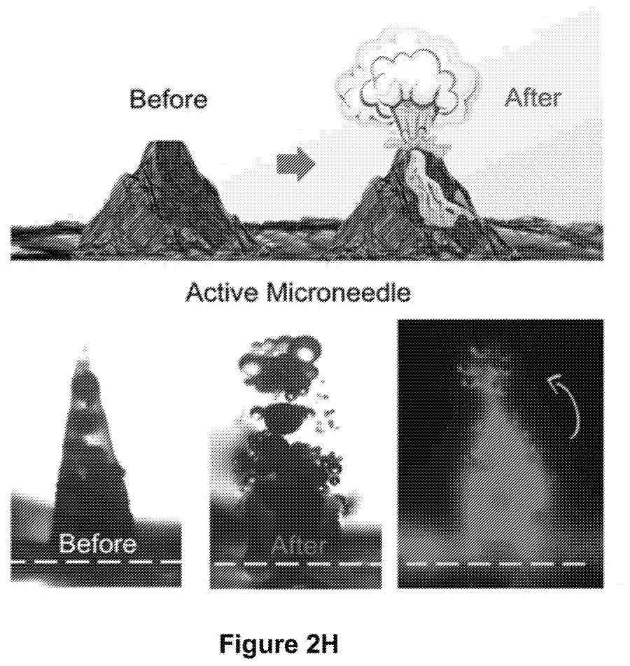

FIGS. 2A-2H shows an example active microneedle for enhanced drug delivery. FIG. 2A shows an illustrative diagram depicting an example embodiment of an active microneedle payload delivery patch 200. FIG. 2B shows an illustrative diagram depicting built-in Mg particle activation as pumps when in contact with bodily fluids, e.g., leading to an enhanced drug release. FIG. 2C shows a panel of fluorescent microscopy and digital photography images of active microneedle tips loaded with Mg particles (with scale bars: 200 μm). FIG. 2D shows a digital photograph (left) showing an example microneedle patch having a 15×15 microneedle array and a scanning electron micrograph (right) of active microneedles of the array (with scale bars: 5 mm (left image) and 400 μm (right image). FIG. 2E shows a three dimensional height microscopy profile image of featuring an example 3×3 microneedle array. FIG. 2F shows an illustrative flow diagram depicting a method 210 for fabricating a microneedle patch device, like the microneedle payload delivery patch device 200, in accordance with the disclosed technology. FIG. 2G shows a panel of microscopy time-frame images taken from a single active microneedle tip clearly showing polymer dissolution and particle activation (before, 30, 60, 90, 120, and 180 s after, respectively), scale bar, 200 μm. FIG. 2H shows time-series images of an example design of active microneedles releasing an encapsulated payload juxtaposed to a graphic illustration of a volcanic eruption.

In some example implementations, a schematic illustration of the active payload delivery microneedle patch 200 is illustrated in FIG. 2A. The active payload delivery microneedle patch 200 includes an array of active microneedle structures 201A that each contain Mg particles 204A as well as the therapeutic payload 203A within a polymer matrix, attached to a substrate 202A. FIG. 2B shows that once the microneedle patch pierces the skin, the polymer microneedle tip starts to dissolve in step 201B exposing the Mg particles 204A surface to surrounding biological fluids and creating an active pump in step 202B. Mg particles 204A are consumed by the interstitial fluid, resulting in the rapid production of H₂ bubbles, thus inducing distinct vortex flow fields at the localized application site in step 203B, which results in the enhancement of the permeation and diffusion of the loaded payload 203A.

The ability to load both active particles and a therapeutic payload in a single needle is illustrated by the fluorescence and digital microscopy photographs shown in FIG. 2C, where the microneedles are loaded with Rh6G (red color) and Mg particles (black dots) (scale bars, 200 μm). The zoom-in view of a single microneedle shows the close packing of the Mg particles in the microneedle structure. FIG. 2D shows a 15×15 needle array illustrating the efficient loading of multiple microneedles (e.g., 400 μm diameter base and 850 μm height, scale bar 5 mm) that preserve a robust hard structure and a sharp, stiff tip (e.g., less than 5 μm), as illustrated in the corresponding scanning electron micrograph (SEM; right, scale bar 400 μm). The consistency in the dimensions of the individual tips of the microneedle array is further supported by the microneedle height profile shown in FIG. 2E.

In some example implementations, active microneedles can be fabricated by a micromolding technique in accordance with the method 210 (FIG. 2F). In this example embodiment, briefly, Mg particles can first be infiltrated into a microneedle mold (e.g., negative PDMS mold) in step 201F, followed by the addition of the therapeutic payload and polymer matrix in step 202F. The polymer can then be dried (e.g., by ambient condition, overnight) in step 203F to form the degradable microneedles in the mold. A final “microneedle patch” can be created by attaching an adhesive side of a substrate to the formed degradable microneedles, and which can be released from the mold in step 204F and stored, e.g., at room temperature (25° C.).

The active microneedles can react quickly after contacting fluids, as shown by the time-lapse microscopy images in FIG. 2G. The images in FIG. 2G were, taken from a single active microneedle tip fabricated according to the methods shown in FIG. 2F and show polymer dissolution and particle activation (before, 30, 60, 90, 120, and 180 s after, respectively scale bar, 200 μm). The dissolution and hydrogen generation of a single active microneedle tip is dissolved in less than 60 sec. These example results are further corroborated by the microscopy time-frame images shown in FIG. 2G polymer showing the dissolution and particle activation (before, 30, 60, 90, 120, and 180 s after, respectively, scale bar, 200 μm). FIG. 2H further illustrates the dissolution of the microneedles upon contact with fluids with images of the microneedle before and after contract with fluids.

### Characterization, Activation, Life-Time Performance and Mechanical Testing of an Example Microneedle Patch

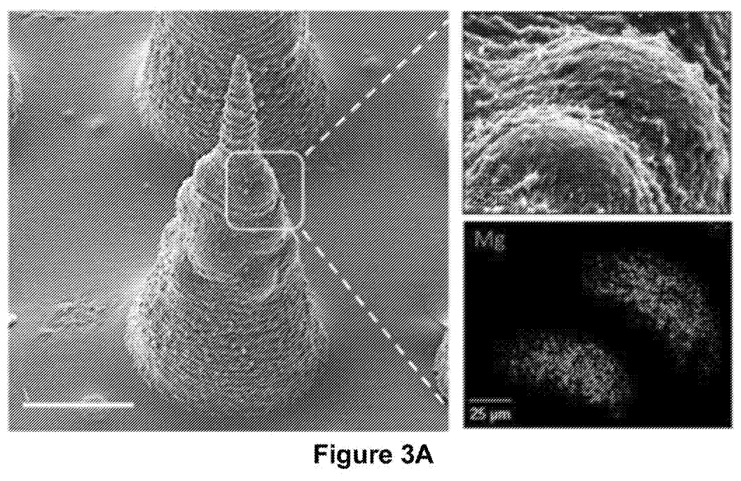

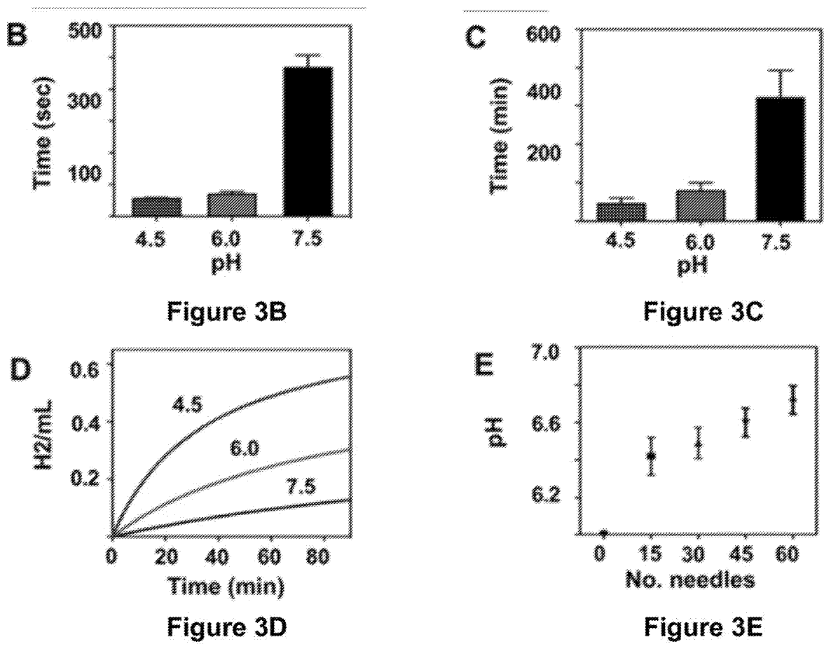

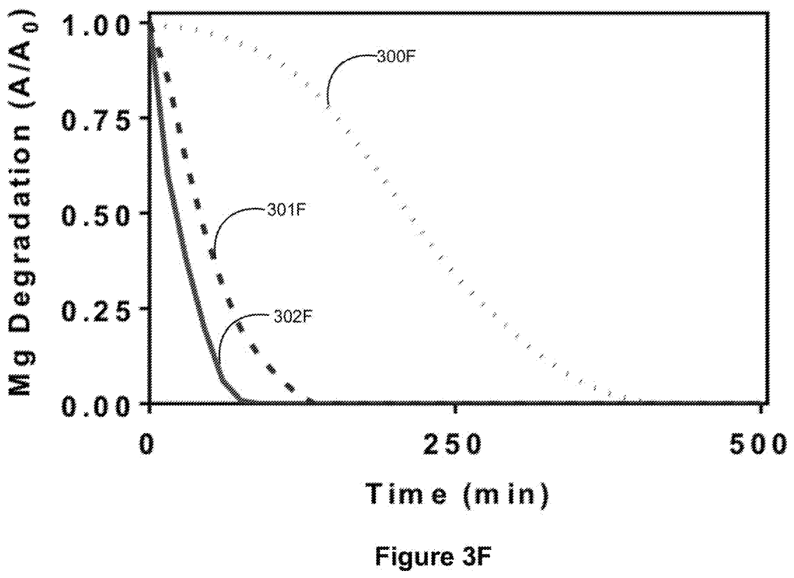

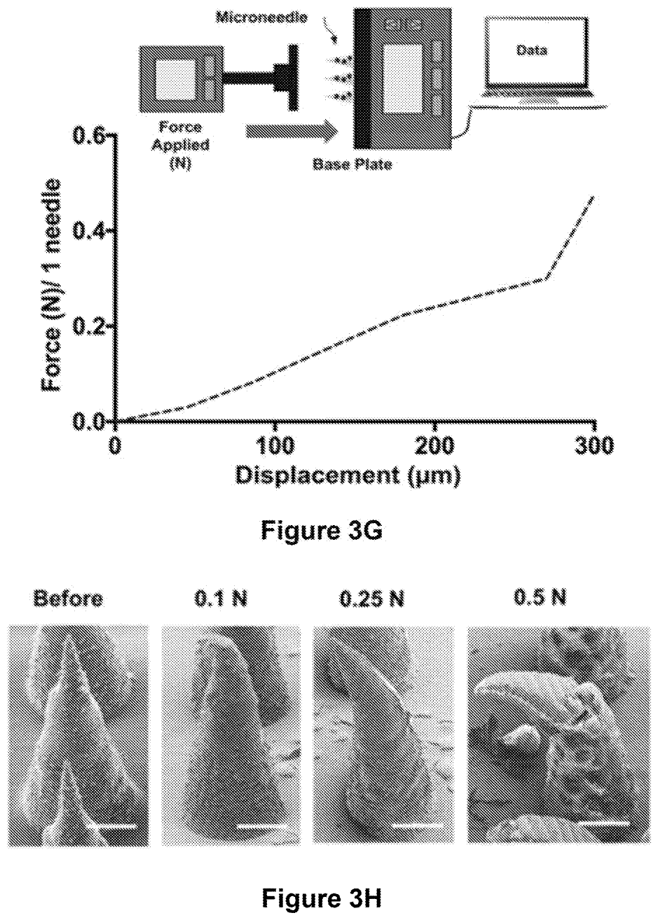

In some aspects, the presently disclosed technology has several advantages such as faster release kinetics, painless application, low cost, sharp tip, no sharp waste and easy of fabrication and practical use. The microneedle structure can have diverse dimensions and shapes, such as a 400 μm base and 850 μm height and have a sharp stiff tip similar to a bee sting (e.g., less than 50 μm). The microneedles can be filled with Mg particles, where polymer (e.g., PVP) casting and polymerization is subsequently carried out on top of the Mg particles, embedding the Mg particles in a polymer matrix. The outcome of this process allows the formation of shaped microneedle tips (FIGS. 3A-3H).

In some example implementations, the parameters relevant to the Mg active-delivery performance were examined to understand the active microneedles under different environments. The enhanced mixing of the active needle is dependent on the number of magnesium particles loaded. Based on findings that the peak value of random sphere packing of conical structures is 66.64%, it is estimated that a microneedle array that includes a total of 225 microneedle tips can load a total volume of ˜10.13 μL in the tips, thus providing flexible delivery possibilities. The theoretical volume loading capabilities of a single microneedle tip was calculated and plotted as shown in FIGS. 1C-1D

FIGS. 3A-3H show example active particle microneedle performance, activation, degradation and mechanical testing. FIG. 3A shows a SEM image of an example embodiment of a single active microneedle tip containing Mg particles, and a corresponding energy dispersive x-ray spectroscopy (EDX) image of the Mg embedded within the example embodiment of the single active microneedle tip (scale bar, 400 μm and 25 μm, respectively). FIG. 3B shows a data plot depicting the time necessary for an example embodiment of active microneedle tip containing Mg particles to start reacting (n=5). FIG. 3C shows a data plot depicting the degradation time of an example embodiment of active microneedle tip containing Mg particles (n=5). FIG. 3D shows a data plot depicting the rate of H₂ generation induced by the active microneedle tip containing Mg particles in the presence of different pH environments. FIG. 3E shows a data plot depicting the pH solution variation of an example embodiment of active needles after complete dissolution (15 min). FIG. 3F shows a graph of the Mg particle degradation for an example embodiment of an active microneedle Mg. FIG. 3G shows a graph and a schematic of depicting the mechanical testing of an example embodiment of the microneedle patch, the schematic illustrates the experiment and mechanical analysis of the example embodiment of a single microneedle tip under different loads. FIG. 3H shows a panel of scanning electron micrographs of an example embodiment of active microneedle tips before and after the application of a load (scale bar, 200 μm).

In some example implementations, the close packing of the Mg particles within the microneedle is illustrated by an SEM of a single microneedle tip, filled with Mg particles, along with its corresponding EDX, corroborating the Mg successful inclusion within the microneedle structure (FIG. 3A). Furthermore, the parameters relevant to the Mg reaction were tested. The main parameter that affects the Mg reaction is the pH of the biofluid solution as shown in FIGS. 3B and 3C. Low pH solutions (e.g., pH 4 and 6) induced faster activation time and smaller life-time of single Mg particles, when compared to neutral pH which presented delayed activation of the particles and a longer lifetime. The main difference between pH can be attributed to the hydrogen generation, as shown in FIG. 3D, where the H₂ production rate increases with decreasing pH environment. It should be noted that the byproduct of the Mg reaction (protons depletion) results in a local increase of pH, which is shown in FIG. 3E. It is contemplated that such pH dependent actuation could result in tailored therapies and therapeutic modalities.

The degradation rate of the Mg particles in the active and passive microneedles shows a slow degradation rate of Mg (FIG. 3F). FIG. 3F includes a plot 300F representing Mg particles in a pH media of 7.5, a plot 301F representing Mg particles in a pH media of 6.0, and a plot 302F representing Mg particles in a pH media of 4.5. Measurement of the mechanical strength of the active microneedle under tensile compression (FIGS. 3G-3H) displayed a fracture point of 0.5N per needle, corroborating its potential capabilities to pierce skin and scalability for in vivo applications. Microneedle tips were also visualized by SEM before and after the application of different loads (e.g., 0.1, 0.25 and 0.5N), where Mg particles can be seen inside the microneedle structure after fracturing (FIG. 3F).

### Dissolution Rate, Fluid Mixing, and MSD of Microneedles

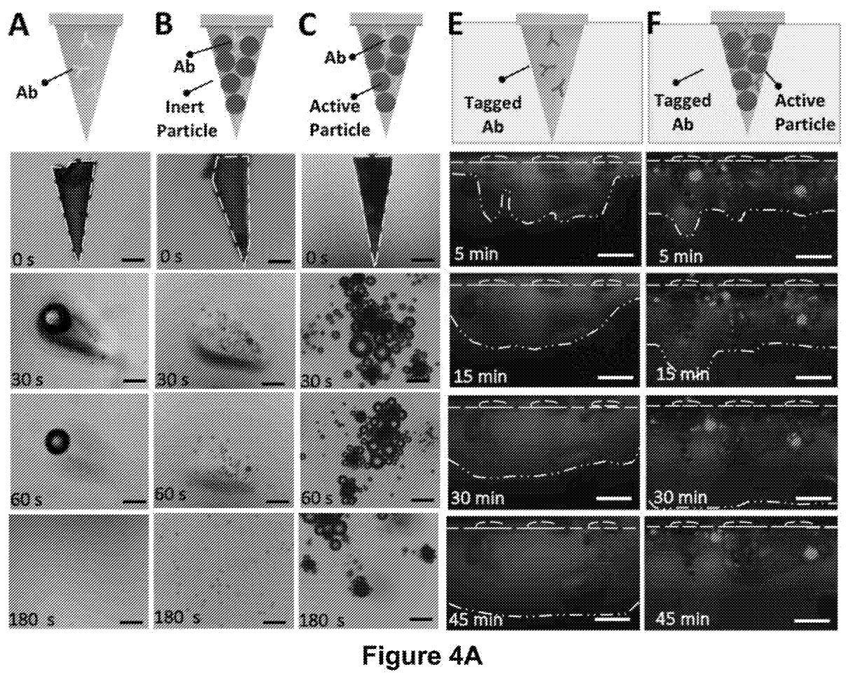

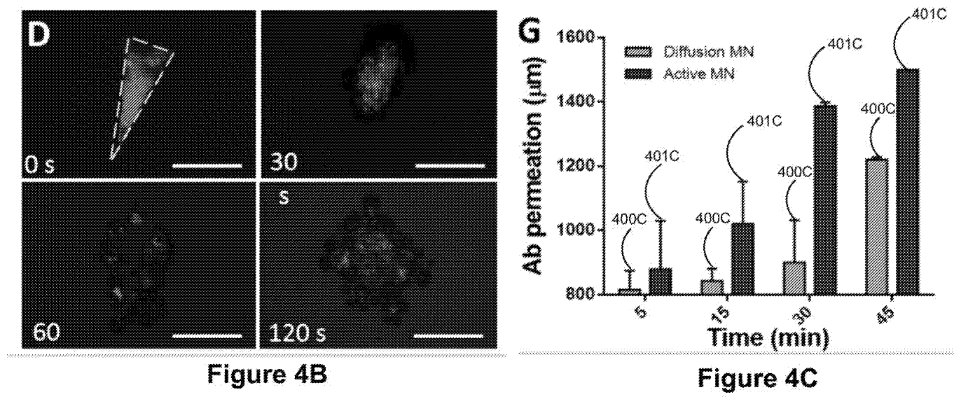

In some aspects, a combination of the presently disclosed transient polymer and active biodegradable particles upon its activation in biological media enhance the delivery of a payload. This technology can work for, but not limited for the enhanced delivery of antibody, protein, enzyme, and drug-small-large molecules. The microneedle dissolution rate changes significantly if active particle microneedles are compared to static and inert particles as well as only polymer microneedles. Mg active particles can pertain activity for more than 30 min in diverse pH environment, generating local fluid transport phenomena in the applied area thus, dissolving faster the microneedle and consequently a greater dispersion of the payload (FIG. 4A-4C). Active particle degradation can be tuned depending on the pH of immersed media, as well as change local pH within time.

In some example implementations, prior to performing in vitro release kinetic experiments and further testing of the enhancing capabilities of active microneedles in payload delivery, the fluid mixing performance of the active Mg particles was evaluated in the presence of smaller tracer particles in solution. The polymer dissolution of different microneedle tips, by the variation of active particles, inert particles, or without any, varied as expected.

FIGS. 4A-4C show time-lapse images of the dissolution rate, fluid mixing, and permeation of antibodies embedded within different microneedles of an example embodiment of a degradable microneedles patch. In some example implementations, schematic illustration of each microneedle tip used as a control in experiments is shown (FIGS. 4A-AC). FIG. 4A shows example schematic illustrations and time lapse images (in each vertical panel) of the dissolution rate and fluid mixing of an example PVP microneedle under different experimental conditions. Specifically, FIG. 4A shows: at panel A, an example embodiment of a PVP microneedle without an embedded active particle; at panel B, an example embodiment of a PS inert particle PVP loaded microneedle; at panel C, an example embodiment of a Mg PVP loaded active microneedle; at panel E, an example embodiment of a PVP microneedle with a tagged antibody; and at panel F, an example embodiment of a PVP microneedle embedded with tagged antibody and active particles in PBS buffer pH 6 (scale bar, 200 μm). FIG. 4B shows images of depicting the dissolution of an example embodiment of the present disclosure embedded with an antibody and active particles. FIG. 4C shows a bar graph depicting the antibody permeation of example embodiments of microneedles embedded an antibody and active particles (MN active) as compared to the permeation microneedles embedded with an antibody but not an active particle (MN diffusion).

Time-lapse images of the dissolution rate in bare phosphate buffered solution pH 6 of different microneedles are presented as follows: polymer (e.g., PVP) (FIG. 4A, Panel A), polymer (e.g., PVP)+inert PS particles (FIG. 4A, Panel B), polymer (e.g., PVP)+active particles (e.g., Mg) (FIG. 4A, Panel C), antibody (FIG. 4A, Panel E), and active particles (e.g., Mg) tagged with an antibody (FIG. 4A, Panel F). FIG. 4A shows that the microneedle dissolution rate changes significantly upon incorporating the active particles due to the localized active H₂ bubble generation when compared to static particles and bare polymer microneedles. At 60 seconds post-dissolution the conical microneedle structure can still be perceived in images, but not with active microneedles, where active particles enhance and accelerate the dissolution of the polymer (e.g., compare FIG. 4A, Panel B to FIG. 4A, Panel C). As mentioned before, the inclusion of Mg active particles in addition to the production of local fluid transport alters also the PVP degradation time significantly, which as well can be tuned depending on the pH media of which is immersed and change local pH within time (alkalize). The dissolution of the microneedles with antibodies as the active load are further shown in FIG. 4B at 0 seconds, 30 seconds, 60 seconds, and 120 seconds after contact with the fluid. FIG. 4C shows the permeation of the antibody into the fluid with active microneedles 400C and passive microneedles 401C at 0 seconds, 30 seconds, 60 seconds, and 120 seconds after contact with the fluid. As shown in FIG. 4C, the permeation of the antibodies for the active microneedles is greater as compared to the permeation of passive particles at each time point.

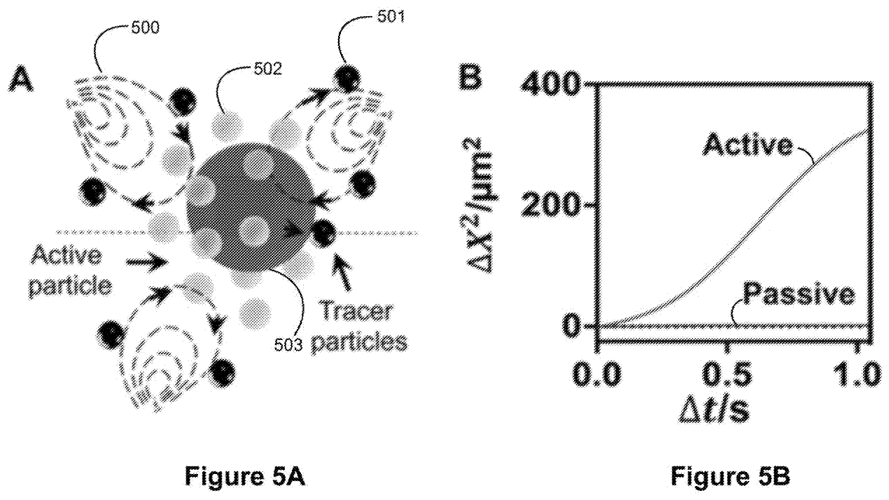

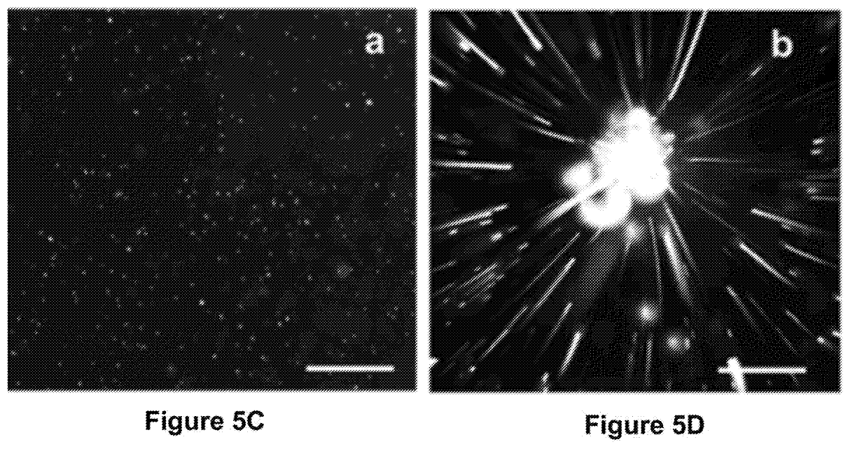

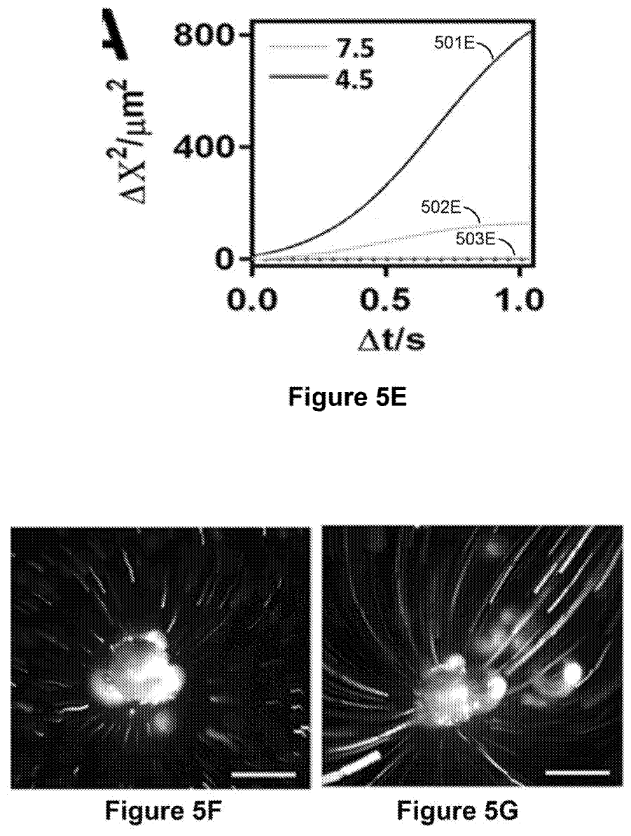

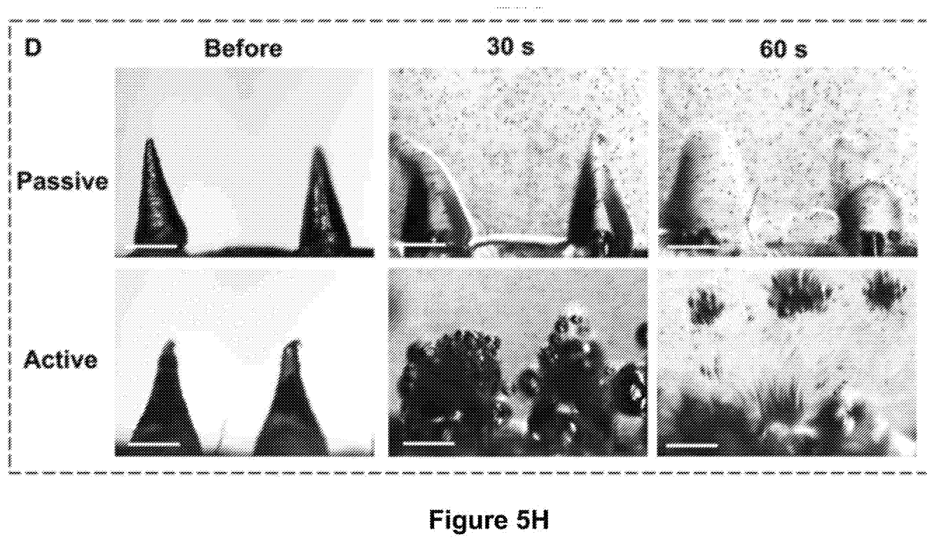

FIGS. 5A-5H show example results from implementations pertaining to the active fluid transport of example active microneedles for enhanced drug delivery. The example data depicted in FIGS. 5A-5F show example active fluid transport (localized streaming) based on Mg particles embedded in dissolvable microneedles. Specifically, FIG. 5A shows an illustrative schematic of the fluid transport of an example embodiment of active Mg particles in the presence of tracer particles. FIG. 5B is a graph depicting the mean square displacement of tracer particles as Brownian motion as control (passive) or in the presence of an example embodiment of active Mg particles at pH 6.0 (active) (scale bar, 100 μm). FIG. 5C shows flow trace images of the trajectory (is) of 0.9 μm polystyrene tracer particles at pH 6.0 without (a) the presence of active particles (Brownian motion), and with active particles (b) at pH 6.0 (Scale bar, 100 μm). FIGS. 5E-5G, similar to FIGS. 5B-5D, show the mean square displacement of tracer particles as Brownian motion of the passive vs. active particles and the corresponding tracer images, respectively. FIG. 5H shows time-lapse images of two example embodiments of microneedle tips without active particles showing the dissolution of the transient polymer by diffusion (a) and loaded with Mg particles accelerating the dissolution of the transient polymer due to their fast activation (b) (scale bar, 400 μm).

In some example implementations, the embedded Mg particles were found to substantially increase the displacement of tracer microparticles through fluid convection resulting from their microbubbles production. A schematic of the fluid transport of Mg active particles in the presence of tracer particles is presented in FIG. 5A. FIG. 5A shows the Mg active particles including Mg particles 503 and active particles 502 immersed in a solution containing 0.9 μm fluorescent beads used as tracer particles 501 to compare the corresponding flow trace 500 capabilities of each Mg particle 503 at different pH environment.

The Mean Square Displacement (MSD) of the tracer particle trajectories, in the absence and presence of the Mg particles 503, is shown in FIG. 5B. FIG. 5B shows the Brownian motion as a control (trace labeled passive) or in the presence of the active Mg particles 503 (trace labeled active) at pH 6.0. The data demonstrates that there is a significant increase in Brownian motion due to the induced built-in mixing compared to the passive diffusional conditions. Flow trace images of the trajectory over 1 s at pH 6.0 is shown in FIGS. 5C-5D, in the absence of Mg particles (Brownian motion) as a control FIG. 5C, and with Mg particles FIG. 5D (scale bar 100 μm). FIGS. 5E-5G show similar data using a variable pH between 4.5-7.5.

FIGS. 5E-5G similarly show MSD of active Mg particles at different pH levels of 4.5 and 7.5. FIG. 5E includes plot 501E depicting the MSD of active particles at pH 4.5, a plot 502E depicting the MSD of the active particles at pH of 7.5, and a control plot 503E. Time lapse images of the flow trace of 0.9 μm fluorescent beads used as tracer particles at pH 4.5 and 7.5 are shown in FIGS. 5F and 5G, respectively. (scale bar 100 μm).

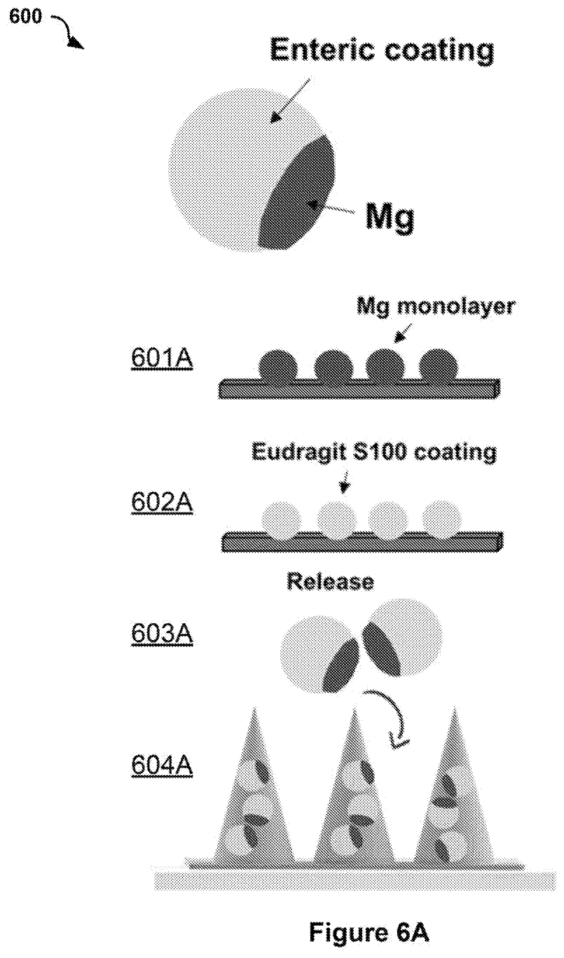

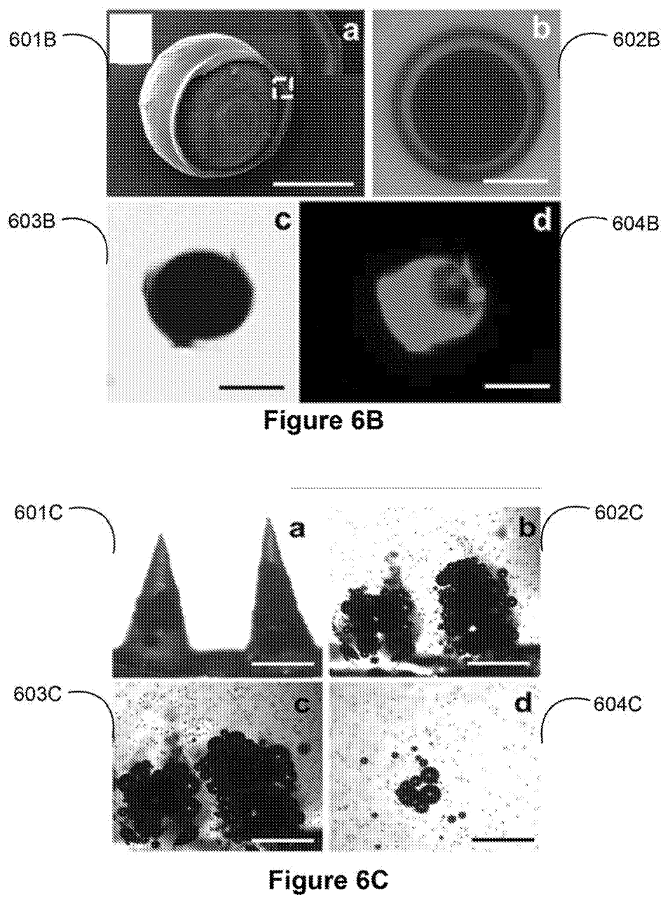

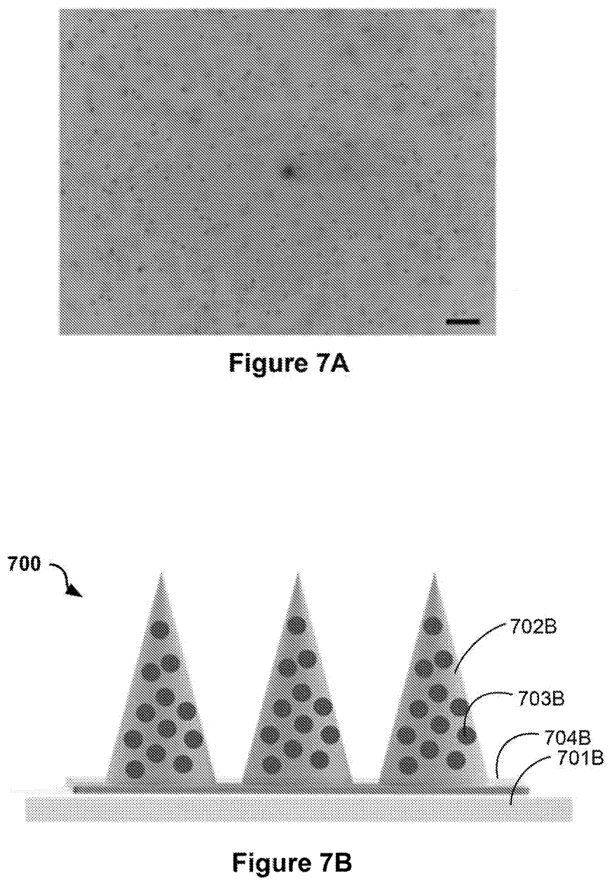

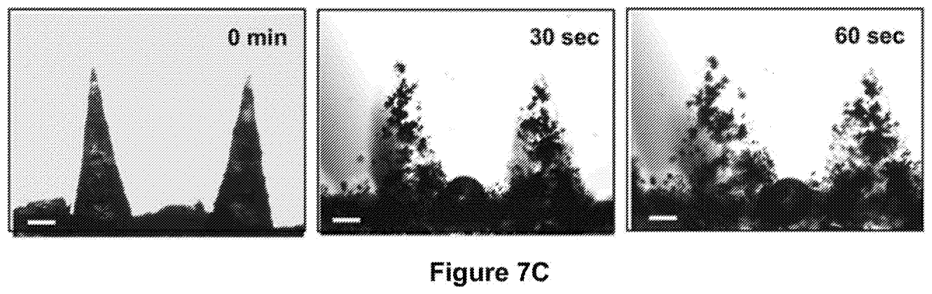

FIG. 5H shows time-lapse images of two microneedle tips without and with Mg particles. FIG. 5H shows that localized mixing due to the previously loaded particles accelerates the dissolution of the polymer significantly due to faster activation of the particles, but more notorious is the accelerated collision of the tracer particles in solution compared to passive microneedles. In some implementations, active microneedles can be loaded with Rhd6G, which can result in accelerated dissolution and Mg particle activity. In some implementations, the active microneedles can be tuned so as to delay the activation of the particles. For example, to tune the capabilities of Mg particles to control a delayed activation and to highlight the high localized fluid mixing these 50-100 um Mg particles pertain, studies have been developed showing that the use enteric coating on the surface of the particles, as well as the inclusion of smaller Mg particles within microneedles can influence the rate of activation as represented in FIGS. 6A-6C and 7A-7C, respectively.

FIGS. 6A-6C show illustrative diagrams and example data from implementations using an active microneedle for enhanced drug delivery where the embedded Mg particles are coated with an enteric coating. In FIGS. 6A-6C, example embodiments of microneedles embedded with Mg particles are shown to be tuned so as to control a delayed activation of the particles. FIG. 6A shows an illustrative schematic depicting an example fabrication process of an example embodiment of an active microneedle with Mg particles coated with an enteric coating. FIG. 6B shows a panel of SEM and fluorescent images of an example embodiment of Mg particles coated with an enteric coating (scale bar, 50 μm). FIG. 6C shows a panel of time lapse images of an example embodiment of microneedles with embedded Mg particles coated with an enteric coating (scale bars, 400 μm).

FIG. 6A shows a method 600 for coating the Mg particles with an enteric coating to control the delayed activation of the particles. Method 600 includes a process 601A in which a monolayer of Mg particles are formed onto a substrate. Method 600 then includes a process 602A in which an enteric coating of a polymer (e.g., an Eudragit 5100 polymer) (solubility >pH 7.0) is formed over the monolayer of the Mg particles. Next, the method 600 includes a process 603A to release the enteric coated Mg particles from the substrate. The method 600 includes a process 604A to load the (released) enteric coated Mg particles in microneedles to form an array of active degradable microneedles.

FIG. 6B shows SEM images 601B of the enteric coated Mg particles, optical microscopy images 602B and 603B of the enteric coated Mg particles and fluorescent images 604B of the enteric coated Mg particles. FIG. 6C shows time lapse images of two microneedle tips loaded with coated Mg particles accelerating the dissolution of the transient polymer but with less reactivity compared to uncoated ones where image 601C was captured at 0 min, 602C at 30 sec of lapsed time, and 603C at 60 sec of lapsed time (scale bars, 400 μm). Lastly, image 604D is an image of the coated Mg particles showing that coating the Mg particles adds directionality to particles, thus reducing reactivity but extending degradation time.

FIGS. 7A-7C show example active microneedles for enhanced drug delivery loaded with 20 μm of Mg particles. FIG. 7A is an SEM image of 20 μm of Mg particles (scale bar, 200 μm). The SEM image of FIG. 7A shows a flow trace of Mg particles with tracer 20 μm PS particles. FIG. 7B is an example schematic depicting an example embodiment of active microneedles for enhanced drug delivery loaded with 20 μm of Mg particles. FIG. 7B shows a panel of time lapse images depicting the dissolution of an example embodiment of active microneedles for enhanced drug delivery loaded with 20 μm of Mg particles (scale bars, 200 μm). FIG. 7C shows time lapse images of two microneedle tips loaded with 20 μm Mg particles at different time points of 0 min (Panel A), 30 sec (Panel B), 60 sec (Panel C), illustrating the degradation of the microneedles and active release of the activation particles.

The illustrative schematic of FIG. 7B shows microneedles of an example microneedle patch 700 loaded with activation particles (e.g., the 20 μm Mg particles of FIG. 7A), where the microneedle patch 700 includes a substrate 701 comprising a base substrate 701B with an adhesive material 704B on top of the base substrate 701B. In various embodiments of the active microneedle delivery system 100, including the microneedle patch 700, the substrate (e.g., substrate 701) can include, but is not limited to, medical adhesives, and non-dissolving polymeric films such as: polyvinyl alcohol. The microneedle patch 700 includes microneedles 702B disposed on the adhesive material 704B, and comprised of activation particles 703B (e.g., m Mg particles) embedded within each of the microneedles 702B.

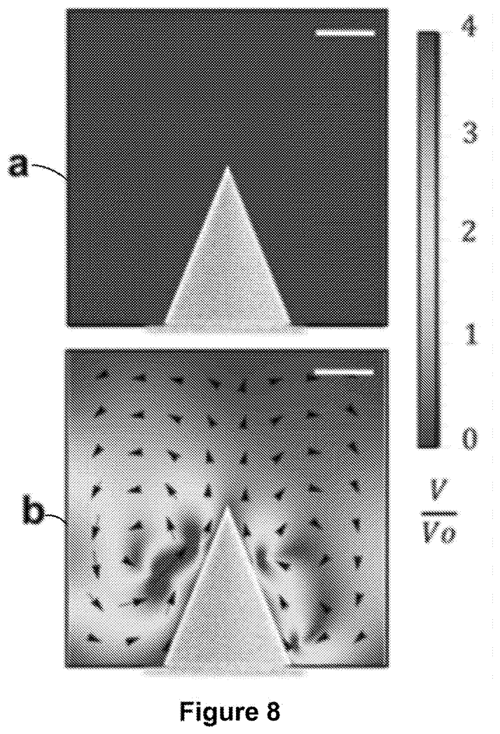

In some example implementations, the accelerated and enhanced localized particle mixing was corroborated by a COMSOL Multiphysics simulation of the flow generated with and without particles in the microneedle structure. FIG. 8 shows the COMSOL Multiphysics simulation of the flow generated without particles (panel (a)) and with particles (panel (b)) within the microneedle structure, for the early stages of microneedle dissolution and activation. Upon reaction of Mg particles with the solution, gas bubbles nucleate and move upward due to buoyancy force. To obtain a simple picture and capture the essential physics of the problem, the effect of bubble motion was approximated by point forces Fᵢ at the location of the ith bubble. While a rough approximation, the results provide a qualitative picture consistent with experimental observations. The fluid field u obeys the Stokes equation

−∇p+∇²u=ΣᵢFᵢ

∇·u=0

where the summation is over the bubbles present in the fluid. As shown in FIG. 8, panel (b) the effect of bubble motions, modeled by point-forces, leads to pumping effect in the fluid. Due to the confined geometry, the fluid follows a circular pattern and results in enhanced mixing. A similar pattern is observed in the experimental set up with tracer particles. Upon the motion of the bubbles, the tracer particles near the substrate move toward the active Mg particle while near the top of the liquid surface, the particles move away from the center.
In Vitro Enhancing Payload Release from Microneedles

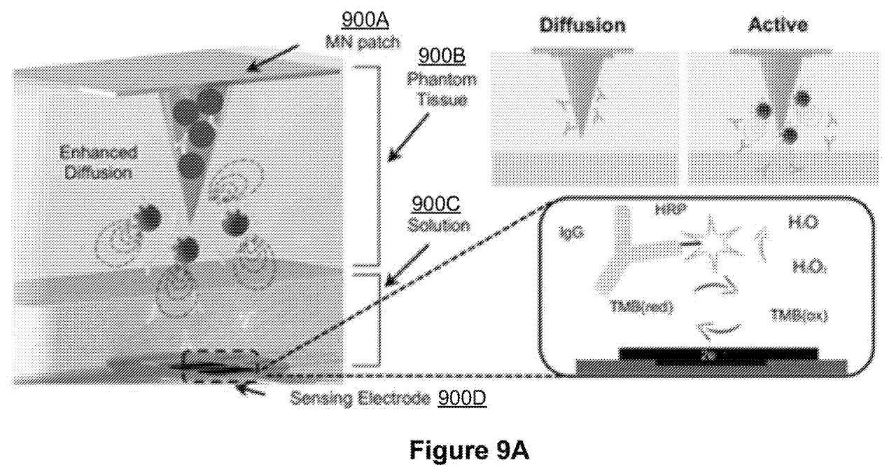

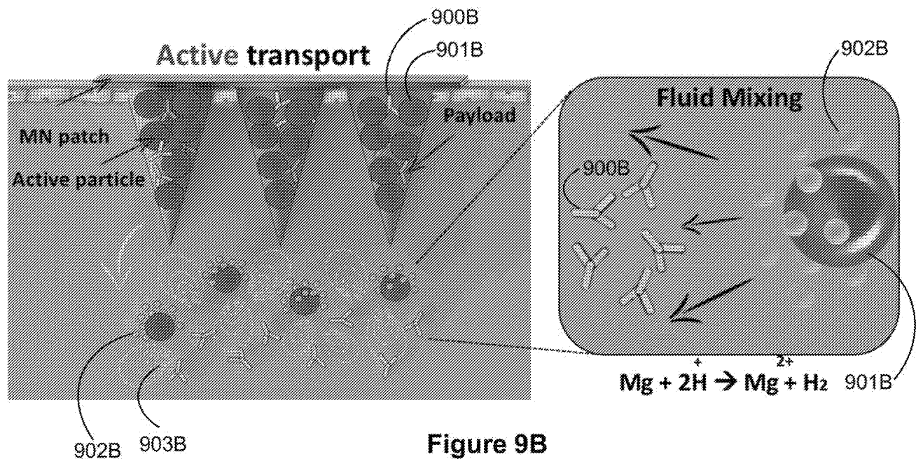

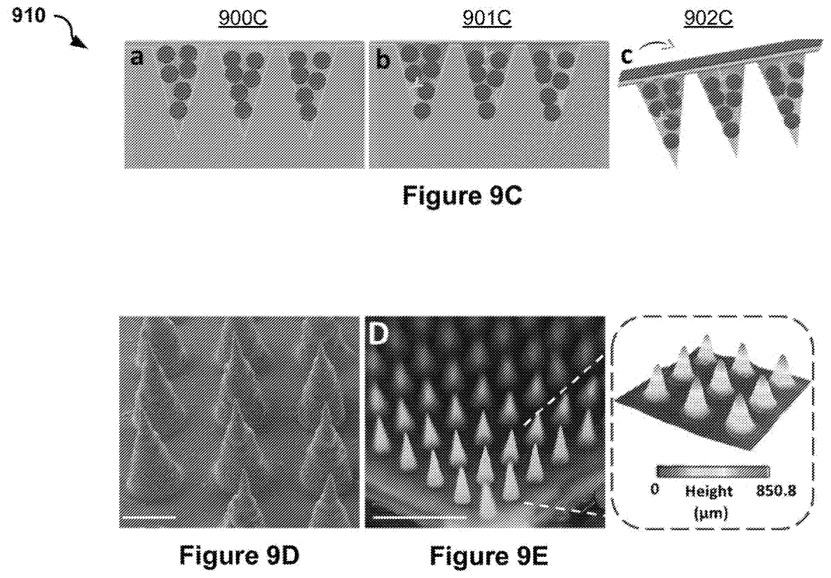

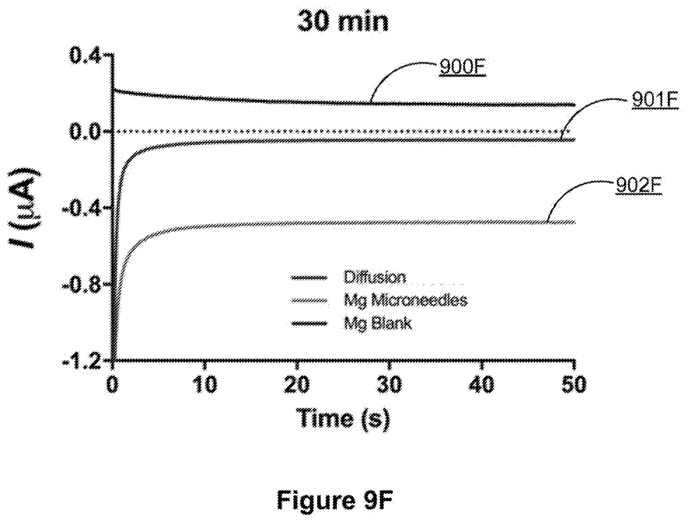

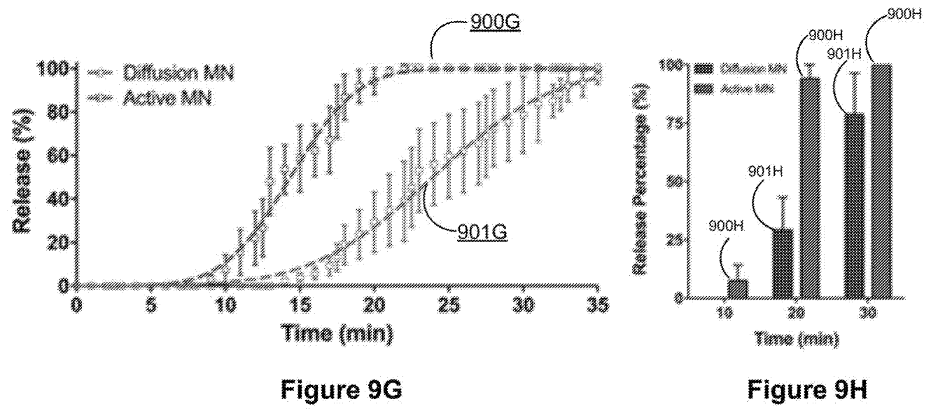

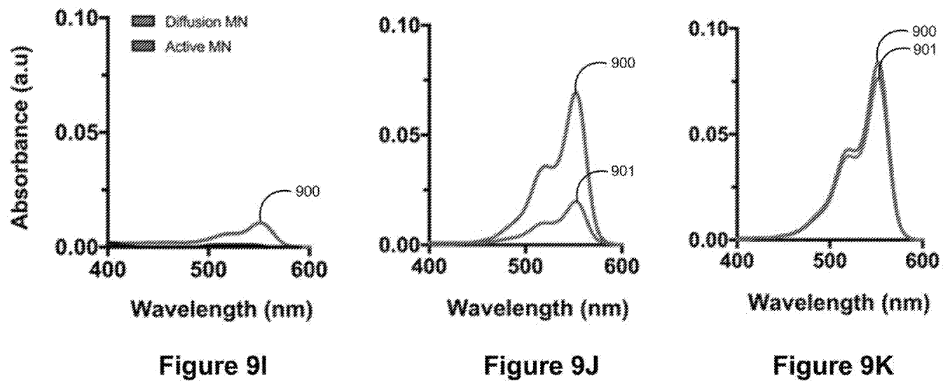

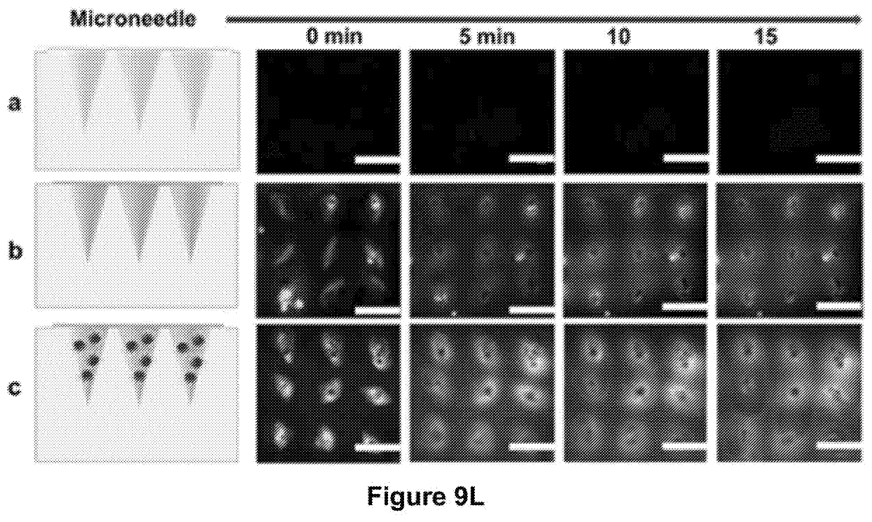

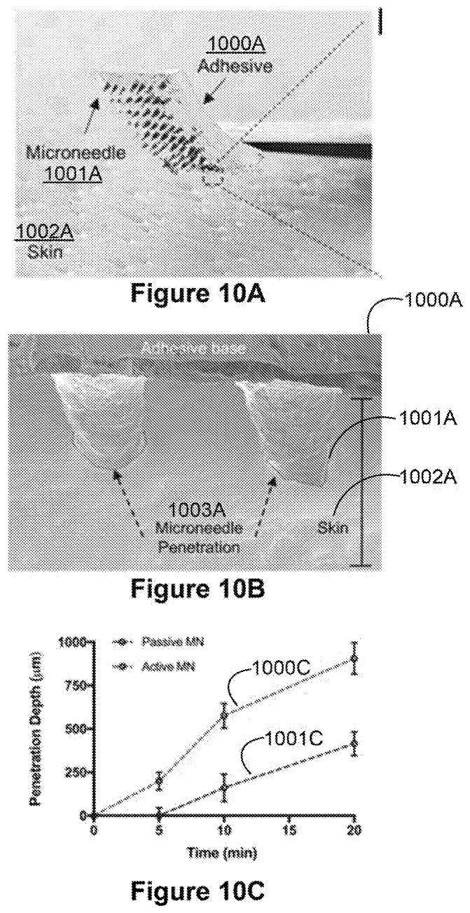

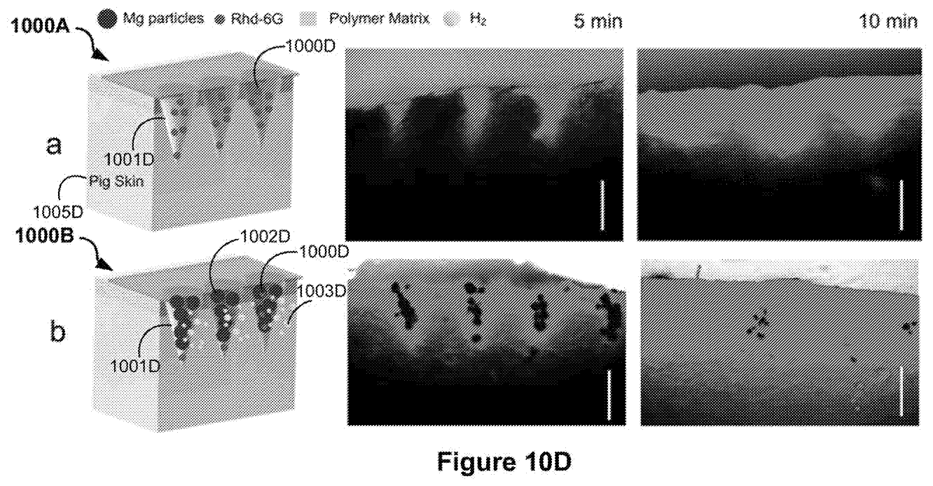

In some implementations of example embodiments of the active microneedle delivery system 100, the activation of the particles for enhancing the dissolution of the microneedle material (faster than normal) was investigated in vitro. It was studied in the example implementations, active microneedles loaded with a fluorescence-tagged antibody improved its distribution and permeation into phantom mimicking tissue when compared to actual diffusion methods (FIGS. 9A-9L). IgG antibody was loaded within microneedles as a payload model and its electrochemical detection was performed to compare the release of diverse approaches. I-t curves were performed for this measurement. The release kinetics of the payload is clearly enhanced for a valuable period of time when the active particle approach is compared to diffusion microneedles (FIG. 10A-10D).

In some example implementations, the in vitro payload release and the potential use of active particle microneedles was evaluated towards enhanced and accelerated therapeutic efficiency. The active microneedle release kinetics were evaluated by employing 3 different techniques: electrochemical, spectrophotometric, and fluorescence. Briefly, for the electrochemical measurements of the payload, active microneedles were loaded with a fixed concentration of 50 μg of a tagged IgG-HRP, this antibody was used as a payload model while its electrochemical detection was performed to compare the release kinetics of diverse approaches.

FIGS. 9A-9L show an example evaluation of the in vitro payload release performance of common passive and active microneedles by fluorescence, electrochemical and spectrophotometric techniques. FIG. 9A shows an example schematic illustrating the electrochemical set up of antibody (Ab) detection based on an amperometric technique. FIG. 9B shows an illustrative diagram depicting built-in Mg particle activation as pumps when in contact with bodily fluids, e.g., leading to an enhanced release of antibodies. FIG. 9C shows an illustrative schematic for a method of fabrication an example embodiment of an active microneedle for enhanced drug delivery comprising antibodies and active Mg particles. FIGS. 9D-9E show a panel of images depicting the tip shape of an example embodiment of active microneedles. FIG. 9F show a graph depicting the release profile of an example embodiment of active microneedles as compared to a control. FIG. 9G shows a graph depicting the release kinetics of corresponding Ab delivery of both example embodiments of passive and active microneedles at pH 6.0 (passive microneedles (PVP and IgG-HRP) and active microneedles (PVP, IgG-RP and Mg particles, n=5). FIG. 9H shows a graph depicting the corresponding release percentage of Ab at different time points, n=5. FIG. 9I-9J shows absorbance spectra of IgG-Alexa Fluor-555 that passed through the phantom mimicking tissue measured at different time points (10, 20 and 30 min) with the use of bare and active microneedles. Ab content was measured from PBS buffer reservoir located below a 1.5 mm of thickness phantom tissue. FIG. 9L shows a panel of depicts as panel of time-lapse fluorescent images (top view) of microneedles placed on top of a 1.5 mm phantom tissue and taken at different time points (0-15 min). Blank microneedles (a), FITC-loaded microneedles (b), and FITC-loaded active microneedles (scale bar, 1 mm).

In some example implementations, the electrochemical set up used for the detection of the payload (e.g., antibodies) (represented in FIG. 9A) included an MN patch 900A, a phantom mimicking tissue 900B (1.5 mm of thickness), a reservoir of PBS buffered solution 900C at pH 6, and a sensing electrode 900D (e.g., screen-printed carbon electrode).

A schematic of the microneedles including an antibody payload are further depicted and characterized in FIGS. 9B-9E. FIG. 9B includes a schematic showing a depiction of the microneedles penetrating that skin where the microneedles include an antibody payload 900B and Mg particles 901B. Upon contact with the skin, the Mg particles are oxidized from Mg⁰ to Mg⁺² after reacting with H⁺, resulting in the production of gaseous H₂ 902B, generating a vortex 903B. The generation of the vortex then permits the mixing of the fluid and the release of the antibody payload into the fluid.

FIG. 9C shows a schematic of a method 910 for fabricating the degradable microneedles comprising a payload substance, such as the antibody payload according to some embodiments of the present disclosure. The method 910 includes a process 900C to provide a mole or template of the microneedle structures and to infiltrate the activation particles (e.g., the Mg particles) in the mold, e.g., a negative polymeric model. The method 910 includes a process 901C to add the payload substance (e.g., antibodies) in the polymeric matrix, added to the Mg particles. The method 910 includes a process 902C to release the payload-, activation particle-loaded degradable microneedles from the mold or template, e.g., where a microneedle patch is created by attaching an adhesive substrate to the base surface of the degradable microneedles and dislodging the microneedles from the mold, e.g., thereby transferring the payload-, activation particle-loaded degradable microneedles to an adhesive base.

FIGS. 9D-9E are images of the resulting microneedles having antibodies as the payload. For the electrochemical detection of the antibody, an amperometric i-t curve technique was performed for this measurement as repetitive runs for a fixed period. The microneedle patch 900A depicted in FIG. 9A was placed over the 1.5 mm in thickness phantom mimicking tissue 900B and further pierced; below the phantom tissue, a reservoir of TMB+H₂O₂ solution 900C was placed for the correct detection of current changes over electrode (e.g., HRP coupled to the antibody); the corresponding amperometry curves at 30 min shown in FIG. 9F.

FIG. 9F shows an amperometry control plot 900F of active microneedles loaded with Mg particles that do not contain antibody (e.g., Mg blank), a plot 901F of passive microneedles loaded with antibody (e.g., conventional microneedle diffusion), and a plot 902F of the release antibody from active microneedles in accordance with the present technology (e.g., containing both Mg and antibody). The release of antibody from active or passive diffusion microneedles that went through phantom tissue can be seen in FIG. 9G.

FIG. 9G includes a plot 900G of the active microneedle particles and a plot 901G of the microneedle particles diffusion (i.e., not active microneedle particles). As shown in FIG. 9G, the diffusion is greater for the active microneedle particles. The results in FIG. 9H show a bar graph depicting the % particle release for the active microparticles particles (900H) and the passive microneedle particles (901H).

FIG. 9H shows an average 15-time fold advantage at the 15 min mark where passive diffusion presents a release percentage of 3.8±1.8% while the active delivery results in 58.5±15.1% release; similarly, the active delivery results in a 3-time fold at 20 min, with 29.3±13.8% bare vs. 94.1±5.8% active. Active microneedles were loaded with IgG Alexa Fluor-555 in order to corroborate the enhanced payload distribution and permeation into phantom mimicking tissue when compared to those of passive (diffusion-based) microneedles. Similar to the electrochemical measurements, setup including a phantom mimicking tissue 900B of a thickness of 1.5 mm and pierced with a microneedle array.

FIGS. 9I-9K show the corresponding absorbance spectra of IgG-Alexa Fluor-555 that passed through the phantom mimicking tissue measured at different time points (10, 20 and 30 min) with the use of bare and active microneedles. FIGS. 9I-9K include a plot 900 representing the active microneedle particle absorbance and a plot 901 representing the diffusion of the passive microneedle particles (i.e., particles not embedded within the microneedle). The antibody content was measured from PBS buffer reservoir located below a 1.5 mm of thickness phantom tissue 900B (see FIG. 9A).

In some example implementations, in FIG. 9L, microneedles were loaded with a relatively low molecular weight molecule (FITC) and the enhanced payload delivery (lateral diffusion) with active microneedles was visualized via fluorescence images (top view) by piercing the microneedle array into a 1.5 mm in thickness phantom tissue. Active microneedles were compared against passive microneedles, and as expected, the top view images taken at different time intervals (e.g., 0, 5, 10 and 15 min) corroborate the accelerated diffusion of FITC significantly at the mark of 10 min.

FIGS. 10A-10D show an example evaluation of ex vivo dye release performance of passive and active microneedles. FIG. 10A shows a digital photograph of an example embodiment of an active microneedle patch (7×7 array) loaded with Rhd-6G before piercing porcine skin. FIG. 10B shows images of colored scanning electron micrograph of an example embodiment of 2 active microneedles piercing porcine skin. FIG. 2C shows graphs depicting the corresponding penetration depth of Rh-6G by microneedles at different time points, n=3. FIG. 10D shows an example schematic illustrating the experimental set up of both passive and active microneedles penetrating into porcine skin. Passive (a) and active (b) microneedle arrays are shown respectively, as well as fluorescence microscopy cross section images at different times (scale bars, 500 μm).

In some example implementations, an evaluation of the dye release performance of both passive and active microneedles was performed ex vivo onto porcine skin. FIG. 10A, illustrates a digital photograph of an active microneedle patch 1001A including 49 needles (7×7) on top of an adhesive base 1000A, the microneedle patch 1001A is loaded with the dye Rhd6G, before piercing the porcine skin 1002A. Porcine skin 1002A was previously fixed with glutaraldehyde before piercing with microneedles in order to characterize the piercing capabilities. A colored scanning electron micrograph is illustrated in FIG. 10B, showing the successful penetration of two tips into deep tissue. FIG. 10B shows the active microneedles 1001A attached to the 1000A adhesive base penetrating the skin 1002A. The depth penetration 1003A results by piercing porcine skin with both passive and active microneedles was plotted and represented in the graph of FIG. 10C. FIG. 10C includes plot 1000C depicting the penetration depth of the active MN patch as compared to the penetration depth of the passive MN patch depicted by plot 1001C. As show in FIG. 10C, the active MN patch achieves a greater penetration depth as compared to the passive MN patch. The penetration depth was plotted as a function of time, where dye diffused across the skin from the tip. A schematic illustration of both passive and active microneedles piercing into porcine skin is illustrated in FIG. 10D. FIG. 10D shows a passive microneedle array 1000A that includes the microneedle 1001D comprised of a polymer matrix and embedded within the microneedle 1001D is a Rhd-6G dye 1000D where the microneedle 1001D is penetrating pig skin 1005D. For comparison, FIG. 10D shows an active microneedle array 1000B that includes the microneedle 1001D comprised of a polymer matrix and embedded within the microneedle 1001 is a dye Rhd-6G dye 1000D and Mg particles 1002D where the microneedle 1001D is penetrating pig skin 1005D. As depicted by the active microneedle array 1000D, H₂ 1003D is generated due to the presence of the Mg particles 1002D. The microneedle arrays were placed into a porcine skin 1001D rectangular area of 2 cm² and further examined at several time points (e.g., 5, 10 and 20 min). Fluorescence time lapse images of both controls are also shown in FIG. 10D (Right). At the mark of 5 min, it is shown that the entrapped Mg particles 1002D enhanced the delivery of the model dye Rhd6G 1000D by lateral and vertical routes. Such improved permeation is dramatically more pronounced at 10 min, where passive microneedles have an average value of 111±79 μm vs. active microneedles 526±71 μm.

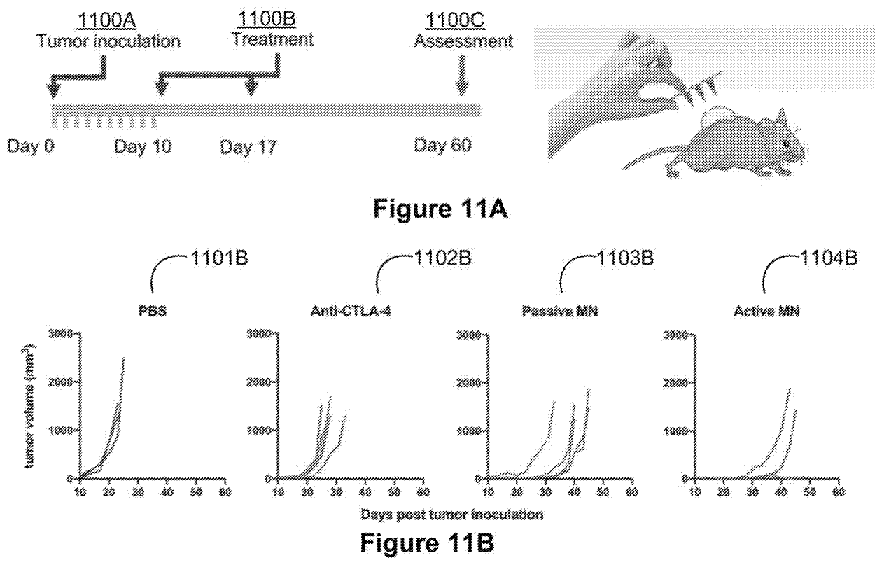

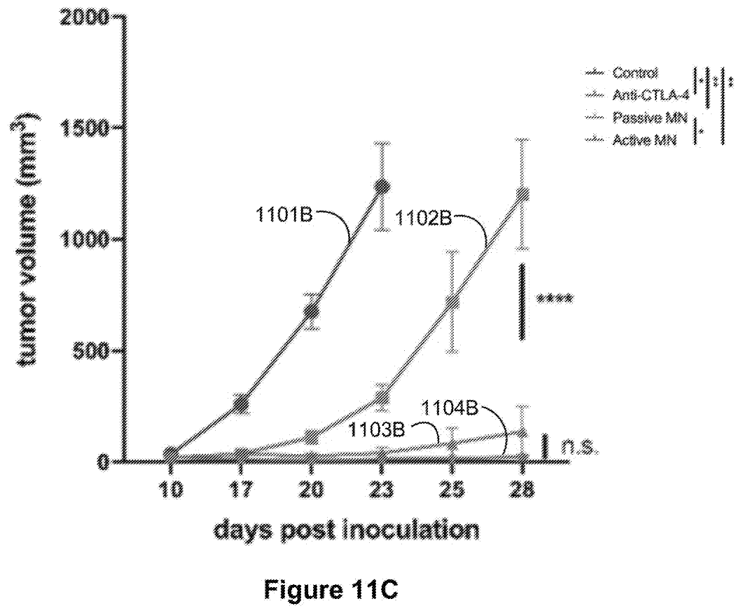

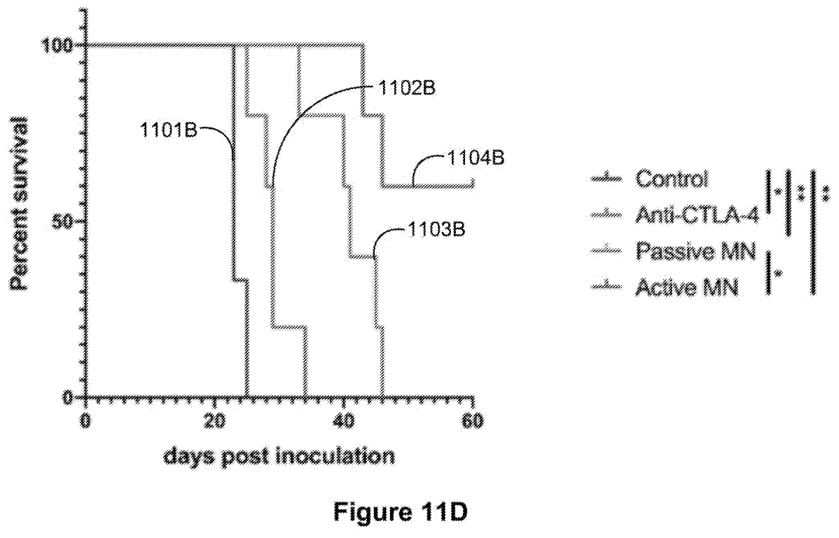

FIGS. 11A-11D show an example in vivo skin cancer treatment using anti-CTLA-4 antibodies delivered by active vs. passive microneedles and intratumoral injection in B16F10 dermal melanoma model. FIG. 11A show an example schematic depicting a study protocol, including tumor inoculation, and treatments. The schematic shows the application of an example embodiment of a microneedle patch applied to a melanoma area in mice. FIG. 11B shows graphs depicting tumor volumes growth curves according to an example embodiment of the present disclosure of individual mice receiving PBS, free anti-CTLA-4, anti-CTLA-4 passive microneedle, and anti-CLTA-4 active microneedle (data are means±SEM (n=3-5)). FIG. 11C shows graphs depicting the averaged tumor volumes of mice under the same controls according to an embodiment of the present disclosure. Tumor growth over time was compared by two-way ANOVA with Tukey's test: ****p<0.0001, n.s. no significant difference. FIG. 10D shows survival rates of the mice after inoculation according to an example embodiment of the present disclosure. Statistical significance was calculated using Log-rank (Mantel-Cox) test: *p<0.05, **p<0.01.

In some example implementations, the in vivo therapeutic efficacy of the active transport method was evaluated by treatment of syngeneic B16F10 mouse melanoma model under different conditions. Anti-CTLA-4 monoclonal blocking Ab was chosen as a model payload to treat melanoma, because it was one of the first immune checkpoint inhibitors tested and approved for cancer patients. Prior to treatment, B16F10 tumor cells were intradermally induced in the right flank of female C57BL/6 mice. FIG. 11A shows a study protocol including tumor inoculation and treatments. The schematic depicted in FIG. 11A includes a tumor inoculation step 1100A from day 0 to day 10, a treatment step 1100B that includes treatment at day 10 and day 17, and lastly, an assessment step 1100C after day 60 (data are means±SEM (n=3-5)). On day 10 and day 17, different treatments were administrated into the tumors.

The immunotherapeutic efficacy of different treatment regimens was assessed by measuring the growth rate of the tumors over time as shown in FIGS. 11B-11D. FIGS. 11B-11D include plots depicting the tumor growth for mice receiving PBS (plot 1101B), an injection of anti-CTLA-4 (plot 1102B), passive microneedles loaded with anti-CTLA-4 (plot 1103B), and active microneedles loaded with anti-CTLA-4 (plot 1104D). FIG. 11B shows the tumor volume growth curves of individual mice receiving each of the aforementioned treatments, FIG. 11C shows the average tumor volumes for mice receiving the same treatments (tumor growth over time was compared by two-way ANOVA with Tukey's test: ****p<0.0001, n.s. no significant difference), and lastly FIG. 11D shows the survival rates of the mice receive the treatments (statistical significance was calculated using Log-rank (Mantel-Cox) test: *p<0.05, **p<0.01). The treatments include injection of PBS shown by plot 1101B, injection of anti-CTLA-4 shown by plot 1102B, passive microneedles loaded with anti-CTLA-4 as shown by plot 1103B, and active microneedles loaded with anti-CTLA-4 as shown by plot 1104B. Free anti-CTLA-4 antibody treatment delayed tumor growth compared to untreated animals (PBS control group plot 1101B). However, both passive microneedles and active microneedles showed significantly enhanced antitumoral effects, achieving significant tumor suppression (FIGS. 11B and 11C, plot 1103B compared to plot 1104B). Passive microneedles delivering the therapeutic anti-CTLA-4 Ab significantly delayed tumor growth. Nevertheless, by day 46 all animals in this group had to be sacrificed due to tumor burden exceeding 1500 mm³. In stark contrast, 60% of the animals treated using the active delivery microneedle platform showed a complete response and were tumor-free and survived past day 50. It is contemplated that the dramatic improved survival is due to the activity of the loaded Mg active particles within microneedles. This built-in pump could enhance the permeation of anti-CTLA through the tumor, therefore improving its distribution to dense metastatic tissue as well as leads to changes in tumor pH (e.g., increased) environment due to the hydrogen depletion of Mg particles.

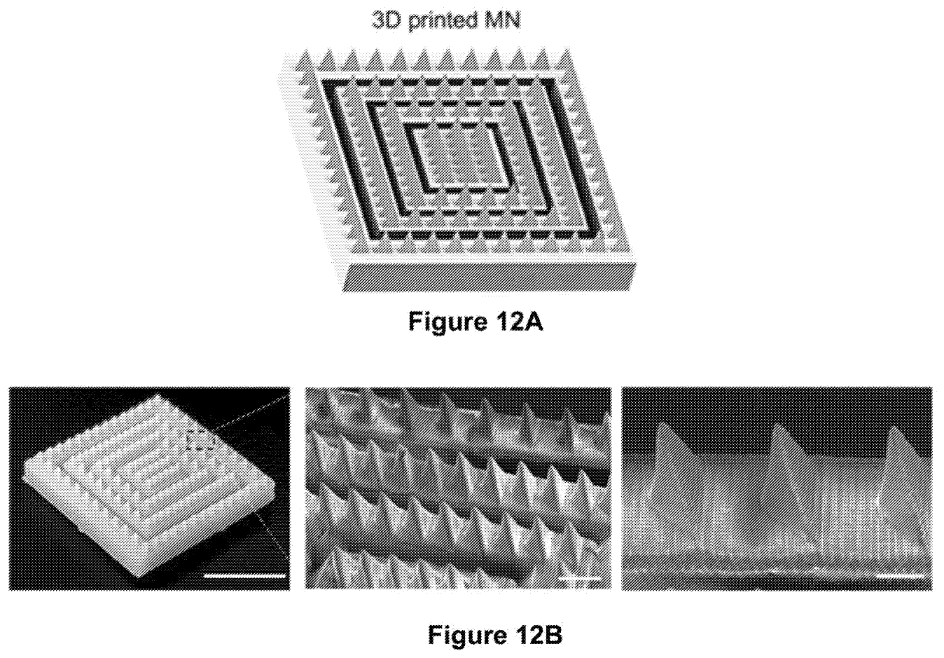

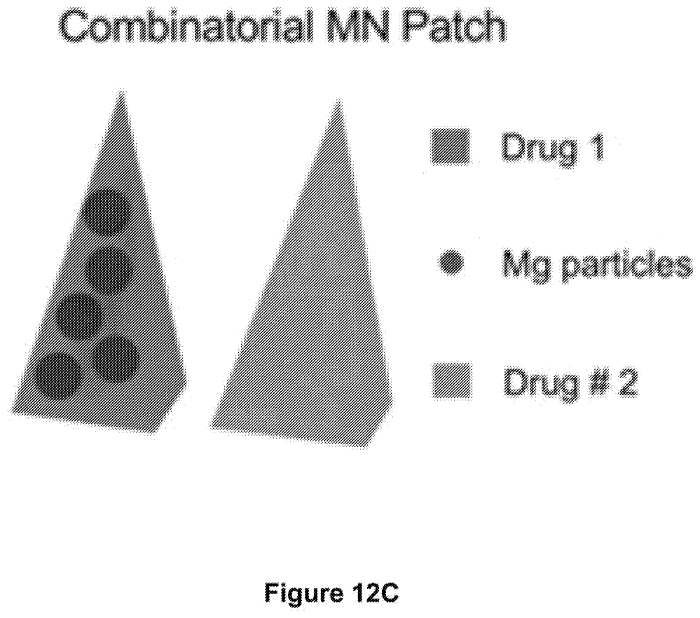

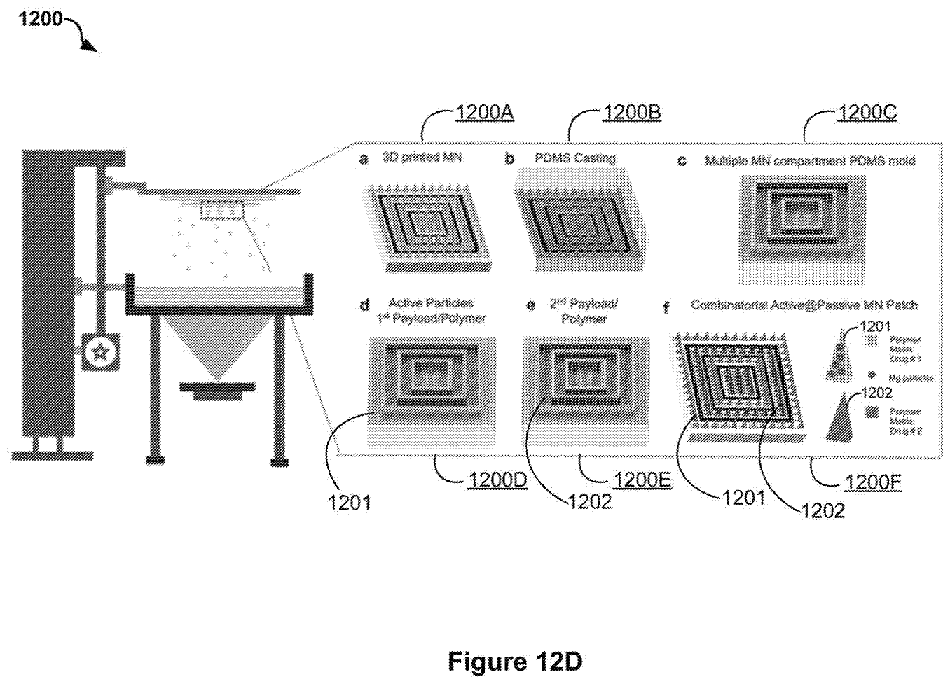

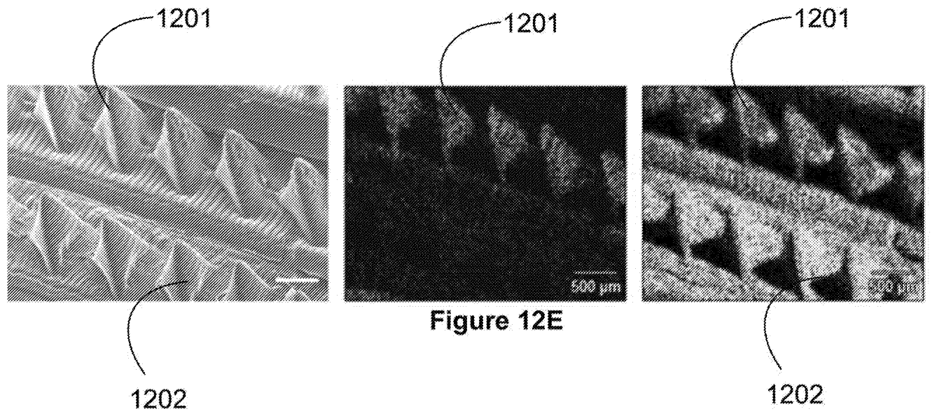

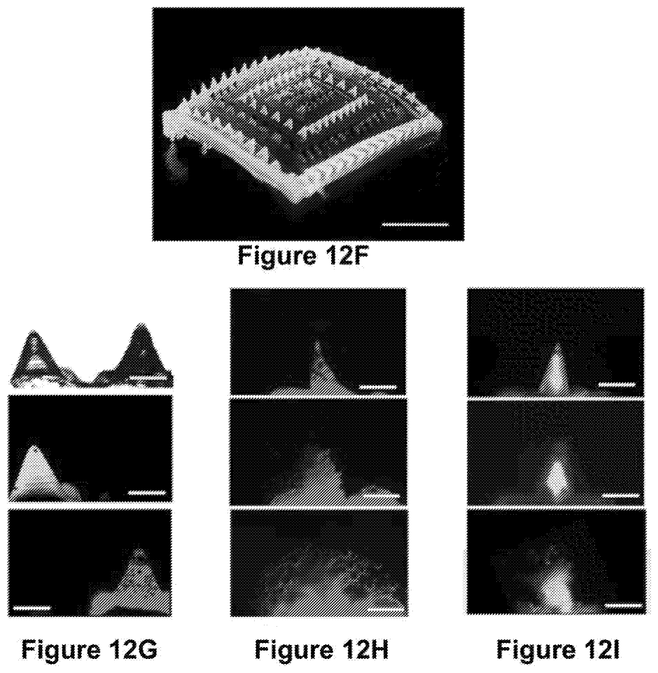

FIGS. 12A-12I show an example combinatorial drug microneedle patch for the simultaneous dual delivery of different payload. FIG. 12A shows an example schematic illustrating an example embodiment of square microneedle arrays as different compartments. FIG. 12B shows a digital photograph of a 3D printed microneedle array by stereolithography and scanning electron micrographs of needle rows with equal spacing (scale bar, 5 mm, 1 mm and 500 μm). FIG. 12C shows an example schematic of a combinatorial dissolvable microneedle patch with 2 different needle compartments (active and passive). FIG. 12D shows an illustrative schematic depicting an example fabrication process of an example embodiment of a combinatorial drug microneedle patch with different compartments. FIG. 12E shows a panel of SEM images depicting side by side active and passive microneedles of an example embodiment of a combinatorial drug microneedle patch; corresponding EDX illustrates Mg (middle panel) and C (right panel) (scale bars, 500 μm). FIG. 12F is a digital photograph of an example embodiment of a combinatorial dissolvable microneedle patch loaded with FITC (passive delivery compartment) and Rhd6G+Mg particles (active delivery compartment) (scale bar, 5 mm). FIG. 12G shows side by side optical and fluorescence microscopy images showing example embodiments of active and passive microneedles (scale bar, 500 μm). FIG. 12H shows fluorescence time-lapse images showing the dissolution of an example embodiment of an active microneedle tip (scale bars, 500 μm). FIG. 12I shows fluorescence time-lapse images depicting the dissolution of an example embodiment of a passive microneedle tip (scale bars, 500 μm).

In the example implementation shown in FIG. 12A, the versatility of the presently disclosed approach is demonstrated by the integration of spatially-resolved active and passive microneedles onto a single patch towards fast/deep and slow sustained combinatorial delivery, respectively. The patch can fabricated by using a stereolithography 3D printer to generate a positive microneedle mold in which topological barriers/walls were engineered to separate groups of microneedles into compartments, thus allowing to combine different materials and cargoes in the same patch design (FIG. 12A). FIG. 12A shows a schematic of square microneedle arrays as different compartments. A digital photograph and scanning electron micrographs of the positive micromold (FIG. 12B) show in detail the distinct groves that separate the microneedles into different sub-sections. FIG. 12B shows digital photographs of a 3D printed microneedle array by stereolithography and scanning electron micrographs (SEM) of needle rows with equal spacing (scale bar, 5 mm, 1 mm and 500 μm). The SEM images of FIG. 12B illustrate in more detail the uniform spacing between such sections, and reproducible tip sharpness. Next, PDMS negative microneedle molds were fabricated and used to fabricate the combinatorial patch following the micromolding method described in the previous sections, but with the added capability of selecting different materials and cargo in each of the microneedle sub-sections (FIG. 12C).

FIG. 12C shows a schematic of a combinatorial dissolvable microneedle path with two different needle compartments (e.g., passive and active). Detailed process fabrication is further illustrated in FIG. 12D.

FIG. 12D depicts a fabrication process 1200 for making combinatorial microneedles according to an embodiment of the present disclosure. The fabrication process includes a step 1200A in which the microneedles are 3D printed onto an array. Next, in step 1200B, a PDMS coating is cast onto the 3D microneedles. Step 1200C shows the formation of multiple microneedle compartments that are formed within the PDMS mold. In step 1200D, the active particles in a first polymer matrix with a first payload 1201 is applied to select compartments of the PDMS mold. In step 1200E, a second polymer matrix with a second payload 1202 is applied to the remainder of the compartments of the PDMS mold. In a final step 120F, a combinatorial (e.g., active and passive) microneedle patch is formed comprised of compartments including active particles 1201 and passive particles 1202.

A scanning electron micrograph of the polymeric combinatorial microneedle patch is shown in FIG. 12D, where both passive needle rows 1202 and active needle rows 1201 are side by side (see FIG. 12E, left panel) and as clearly depicted from the EDX, the Mg particles are only present in the active compartment (see FIG. 12E, middle panel). Each compartment was loaded with model fluorescent payloads. The passive microneedle compartment was loaded with FITC, and the active compartment with Rh6G.

The combinatorial loading of both dyes in a single microneedle patch is further illustrated in FIG. 12F, which shows a digital photograph of the dual compartment under the excitement of UV light. The dual microneedle patch was characterized by fluorescence microscopy, where a side by side image shows both needles located together but with different cargo. In order to visualize the activity of the microneedle tips, FIGS. 12G-12I illustrate fluorescence time-lapse images, showing the dissolution of passive and active microneedles, respectively. It should be noted, that each microneedle is expected to have different release kinetics, were a burst-sustained release profile could help manage acute and chronic pain.

In some embodiments, microneedle patch is a combinatorial path comprising one or more therapeutic payloads. In some embodiments, the one or more therapeutic pay loads include a first therapeutic agent and a second therapeutic agent, wherein the microneedle patch is configured to release the first therapeutic agent in to a subcutaneous fluid prior to a release of the second therapeutic agent.

### Characterization of Plant Virus Nanoparticles for Cancer In Situ Vaccination for Immunotherapy

The solid tumor microenvironment (TME) poses a significant structural and biochemical barrier to immunotherapeutic agents. To address the limitations of tumor penetration and distribution, and to enhance antitumor efficacy of immunotherapeutics, provided herein is autonomous active microneedle (MN) system for the direct intratumoral (IT) delivery of a potent immunoadjuvant, cowpea mosaic virus nanoparticles (CPMV) in vivo.

In some embodiments, the active microneedle delivery system 100 includes magnesium (Mg) microparticles embedded into active microneedles that react with the interstitial fluid in the TME, generating a propulsive force to drive the nanoparticle payload into the tumor.

Example implementations are described showing active delivery of CPMV payload into B16F10 melanomas in vivo, which demonstrated substantially more pronounced tumor regression and prolonged survival of tumor-bearing mice compared to that of passive MNs and conventional needle injection. Active MN administration of CPMV also enhanced local innate and systemic adaptive antitumor immunity. This approach represents an elaboration of conventional CPMV in situ vaccination, highlighting substantial immune-mediated antitumor effects and improved therapeutic efficacy that can be achieved through an active and autonomous delivery system-mediated CPMV in situ vaccination.

Melanoma exhibits high levels of IT heterogeneity, with clonal populations arising due to a variety of selection pressures. For in situ vaccination, it is important to distribute CPMV evenly throughout the tumor tissue to attract adenomatous polyposis coli (APCs) to sample and present tumor antigens from all clonal populations within a single tumor. The MN patches described herein distribute the therapeutic payload over an array of MNs; and thus, offer more uniform delivery throughout the tumor volume. This reduces the reliance on the nonuniform techniques and abilities of administering physicians to disperse the therapeutic throughout the tumor. In addition, CPMV is a macromolecule (30 nm) than most immunotherapeutic agents that have been successfully administered via passive, diffusion-based MNs; thus, active delivery-based administration can enhance CPMV tissue delivery beyond that achieved with passive MNs.

Described herein are autonomous and biocompatible delivery platforms incorporating immunostimulatory CPMV nanoparticles and Mg-based active MN delivery into dissolvable biodegradable MN patches. Provided herein is the characterization of the active MN delivery patch, corresponding spatiotemporal distribution of the payload CPMV, the subsequent immune response, a demonstration of the enhanced therapeutic efficacy of such rapid CPMV release from active MNs for improved in situ vaccination against the B16F10 model of melanoma.

In some example implementations, the active MN delivery system is incorporated with a patch design to facilitate application in treatment of a murine dermal melanoma model. In designing the MN delivery system each of the following were factors considered in order to optimize the system: the materials of which the MNs as composed, the size of the MNs, and the size of the patch serving as the platform from which the MNs extend.

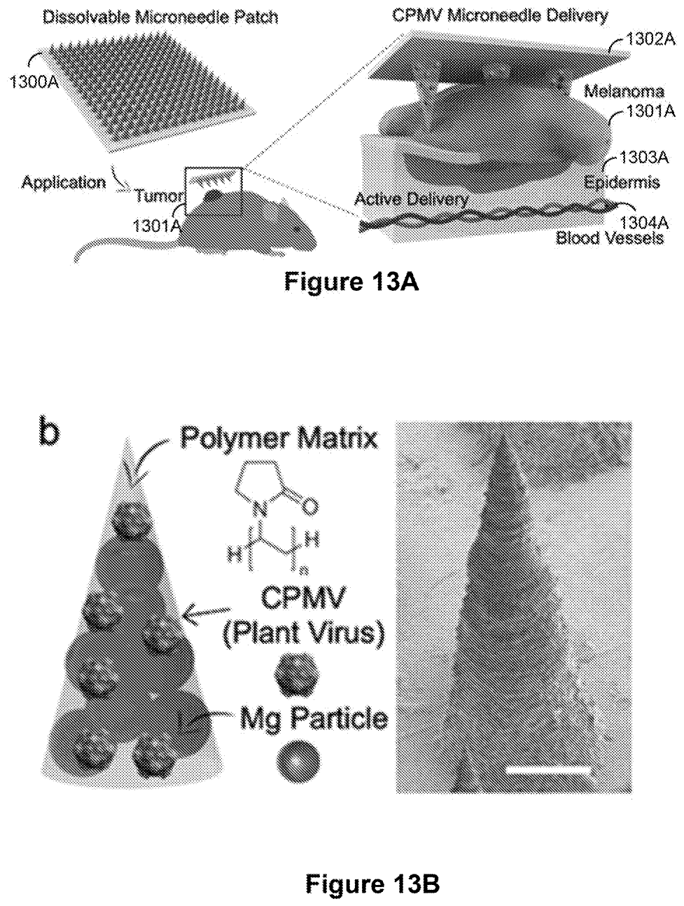

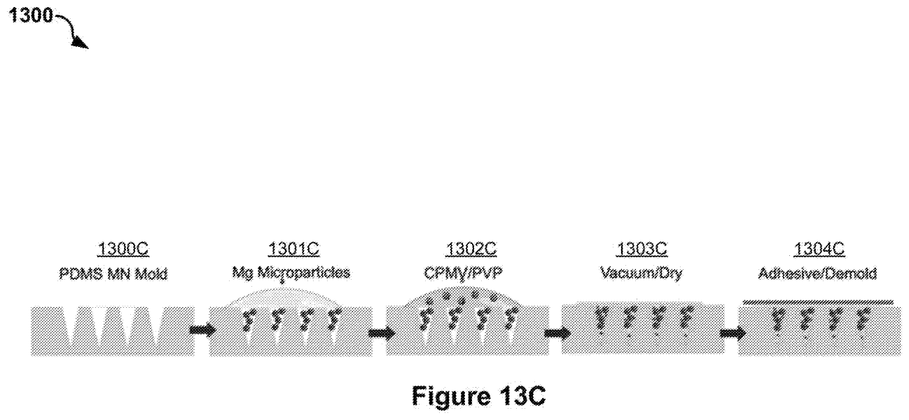

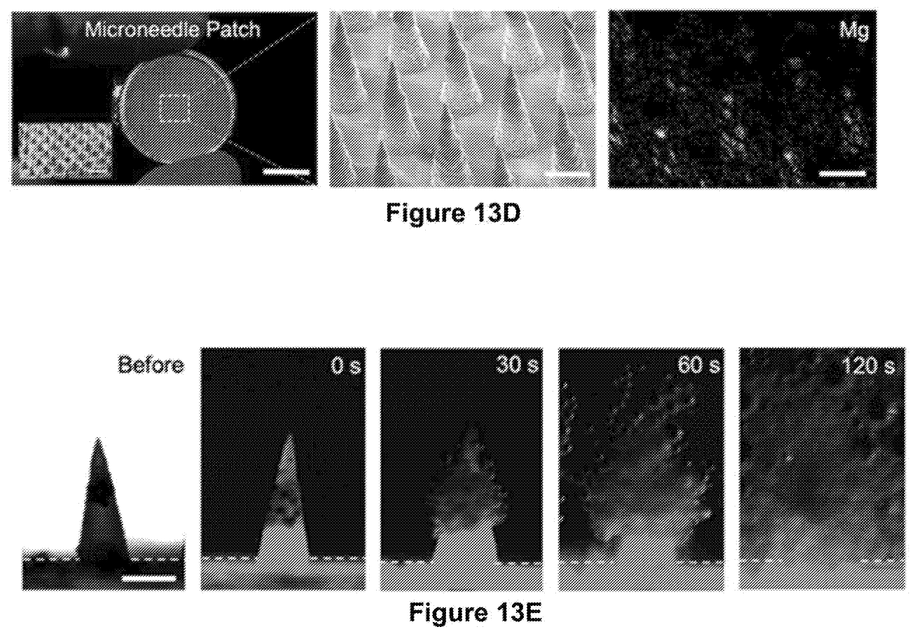

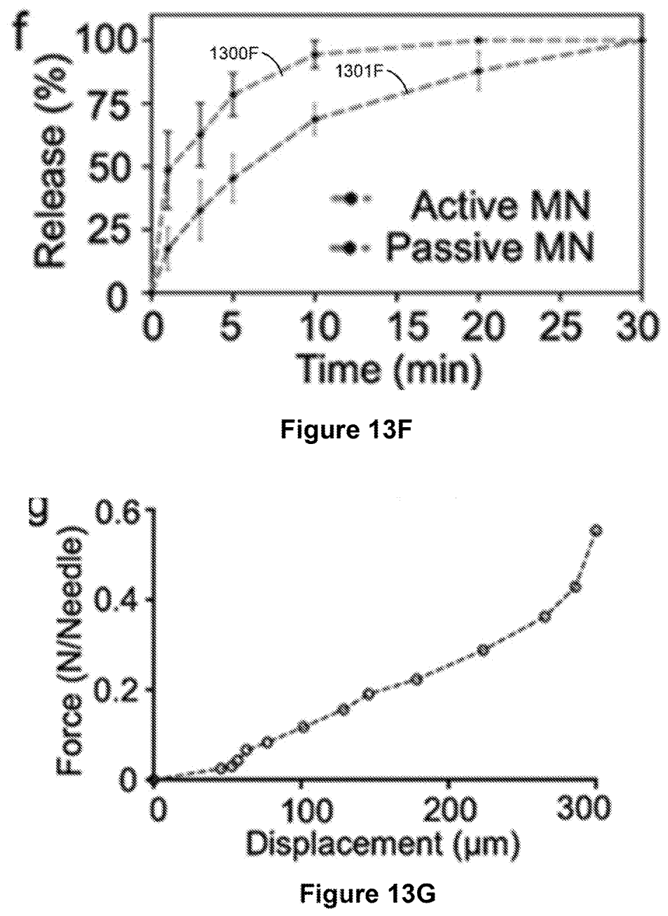

In some example implementations, the active MN patch is comprised of a water-soluble polymer matrix made of a high molecular weight Polyvinylpyrrolidone (PVP), which serves as an enclosure for the active Mg microparticles (e.g., 30-100 μm in diameter) and the therapeutic payload of CPMV nanoparticles, both loaded within the structure (FIGS. 13A-13B). The PVP has been shown to be biocompatible and highly dissolvable with broad use in a variety of biomedical applications.

FIGS. 13A-13B show the formulation of an example embodiment of a dissolvable active microneedle (MN) patch and corresponding characterization. FIG. 13A is a schematic illustrating in situ vaccination with an example embodiment of an autonomous dissolvable active MN patch for the treatment of B16F10 melanoma by the release and delivery of plant virus nanoparticles (cowpea mosaic virus, CPMV). FIG. 13B is a schematic and corresponding SEM image of an example embodiment of a microneedle tip (scale bar, 200 μm). FIG. 13C is an example schematic depicting the steps of fabricating an example embodiment of a dissolvable active MN array to include infiltration of Magnesium (Mg) microparticles onto the negative MN features of PDMS mold, polymer and CPMV loading, drying, and demolding. FIG. 13D shows a digital photograph of a dissolvable active MN patch comprised of 225 MN tips, corresponding SEM image, and Energy Dispersive X-Ray (EDX) elemental analysis of Mg within the active MN tips (scale bars, 5 mm, and 400 μm respectively). FIG. 13E depicts a panel of fluorescent microscopy time-frame images of the dissolution of an example embodiment of an active MN tip, displaying the rapid polymer matrix dissolution and Mg microparticle hydrogen reaction (0, 30, 60 and 120 s intervals) (scale bars, 400 μm). FIG. 13F shows a graph depicting the release kinetics of the delivery of Cy5-conjugated CPMV (Cy3-CPMV) from example embodiments of active and passive MNs. FIG. 13G shows a graph depicting a mechanical strength analysis of an example embodiment of a dissolvable active MN array.

FIG. 13A shows a dissolvable microneedle patch 1300A applied to a tumor 1301A of a mouse. The CPMV microneedle delivery system 1302A then penetrates the tumor 1301A (e.g., melanoma) resting upon the epidermis 1303A without disruption the blood vessels 1304A. The microneedle patches are minimally invasive, and the penetration depth easily controlled. This contrasts with conventional bolus injection of soluble CPMV, where the user variability in terms of how the injections are performed is high and often results in the CPMV injection too deep within the subcutaneous tissue and blood vessels. If the CPMV is injected too deep, this can washout the active ingredient (CPMV) from the tumor through the underlying blood vessel. However, this is avoided using the active MN patch since the length of the microneedles are defined (e.g., <1000 microns long) they are minimally invasive and do not reach subcutaneous tissue nor the blood vessels, preventing washout of the active ingredient. The schematic in FIG. 13B shows the CPMV and Mg particles within a polymer matrix, forming the microneedle.

In some example implementations, the active MN patches comprising CPMV are fabricated by a micromolding process involving a negative polydimethylsiloxane (PDMS) MN molds as reusable templates (FIG. 13C). FIG. 13C shows a process 1300 for fabricating active microneedle patches comprising CPMV according to an embodiment of the present disclosure. The process 1300 includes a step 1300C of obtaining a negative PDMS mold with conical cavities. The process then includes a step 1301C in which the conical cavities of the negative PDMS molds are infiltrated with Mg microparticles followed by a step 1302C in which a polymeric blend of PVP and CPMV nanoparticles are added to the Mg microparticles. In step 1303C, the final patch is obtained by air drying the molds at room temperature. Once dried, the active MN patches were demolded in step 1304C and transferred to medical adhesive base where they are then stored at room temperature (25° C.) prior to application. Passive (control) MN patches were produced in the same manner, except that the Mg loading step was omitted. In some implementations, the MN patches were produced under room temperature conditions and without harsh organic solvents to avoid inactivation or modification of the CPMV.

In some example implementations, the microneedle system includes a circular patch with a thin polymeric base of ˜100 μm in thickness and ˜12 mm in diameter, attached to an array of microneedles (see FIG. 13D, left panel). The MN array size can be designed to accommodate the size of the dermal melanomas on the day of treatment. In some implementations, the array is 15×15 MN array (10 mm×10 mm). In some implementations, the active MN array is comprised of 225 conical-shaped CPMV- and Mg-loaded tips, measuring 400 μm in diameter at the base and 850 μm in length, as shown in a digital photograph in FIG. 13D (left panel). Characterization of MN array structure included scanning electron microscopy (SEM) and Energy Dispersive X-Ray (EDX) elemental analysis to visualize the structure of the MNs and the Mg microparticles contained within them as shown in FIG. 13D (middle and left panel, respectively).

In some implementations, the MN microneedles including the CPMV dissolve upon contact with a fluid. To evaluate the release of CPMV nanoparticles from MNs in vitro, active MN patches were loaded with Cyanine3 dye (Cy3) conjugated-CPMV (Cy3-CPMV). The Cy3-CPMV was distributed within the MN structure and along the base (e.g., a thin polymeric film ˜100 μm in thickness). The Mg microparticles were confined and concentrated within each MN tip. Brightfield (e.g., Nikon Eclipse Instrument Inc. Ti-S/L100) and fluorescence microscopy images (e.g., EVOS FL microscope, RFP fluorescent filter) with immersion in phosphate buffered saline (e.g., PBS, pH 6.5) were then collected. Rapid dissolution of the active MN tips when immersed in solution with vigorous and spontaneous H₂ bubble generation and Cy3-CPMV release was observed (see FIG. 13E). FIG. 13E shows dissolution of an active MN tip, displaying the rapid polymer matrix dissolution and Mg microparticle hydrogen reaction (0, 30, 60 and 120 s intervals, scale bars, 400 μm). The release kinetics and the permeation of Cyanine5 dye (Cy5) conjugated-CPMV (Cy5-CPMV) released by active MNs was then compared to that of passive MNs. Active MN and passive MN patches were applied to a phantom tissue model (˜3 mm thickness) for different durations and Cy5-CPMV release measured by UV-Visual spectrophotometric technique. The release curve results from the active MNs (48.6±15.2%) represented by plot 1300F showed an average 3-fold advantage over the passive MNs (17.4±8.3%) represented by plot 1301F with application for 1 minute. After 5 minutes, the active MNs (78.5±8.4%) showed a 1.5-time fold enhanced release when compared to passive MN (45.3±9.3%) (see FIG. 13F, compare plot 1300F (active MN) with plot 1301F (passive MN)). Finally, the active MNs delivered the full dose 10 minutes after application while the passive MNs had released only a partial dose (68.7±6.0%) (FIG. 13F). The active MNs enhanced and accelerated the Cy5-CPMV delivery relative to that of the passive, diffusion-based MN patch in vitro.

In some implementations the active and passive MNs patches demonstrate a high degree of stability. For example, the active and passive MNs patches can be stable when maintained at room temperature and dry conditions for up to 2 months. The dry polymer PVP matrix can provide stability to both therapeutic payload and Mg microparticles without requiring refrigeration. In some implementations, the MN have a high degree of mechanical stability. Mechanical stability and strength requirements allow the MN to breach dermal barriers in vivo. Accordingly, an axial mechanical compression test on each MN was performed to evaluate its failure force. The mechanical strength results yielded a fracture force of 550 mN per MN tip (FIG. 13G), demonstrating that the active MNs are sufficiently robust to withstand the force necessary for application of the patch to skin.

CPMV In Situ Vaccination with Transdermal MN Patch Application for B16F10 Melanoma

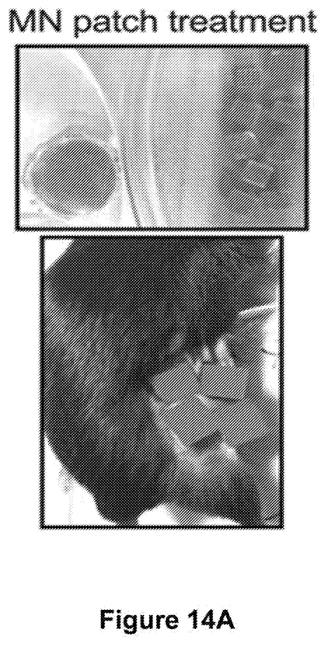

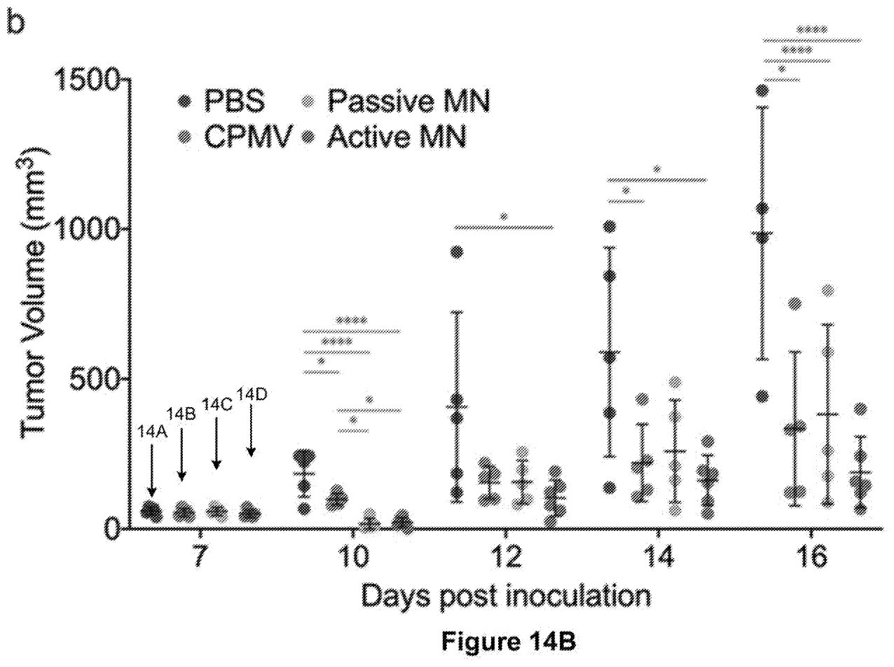

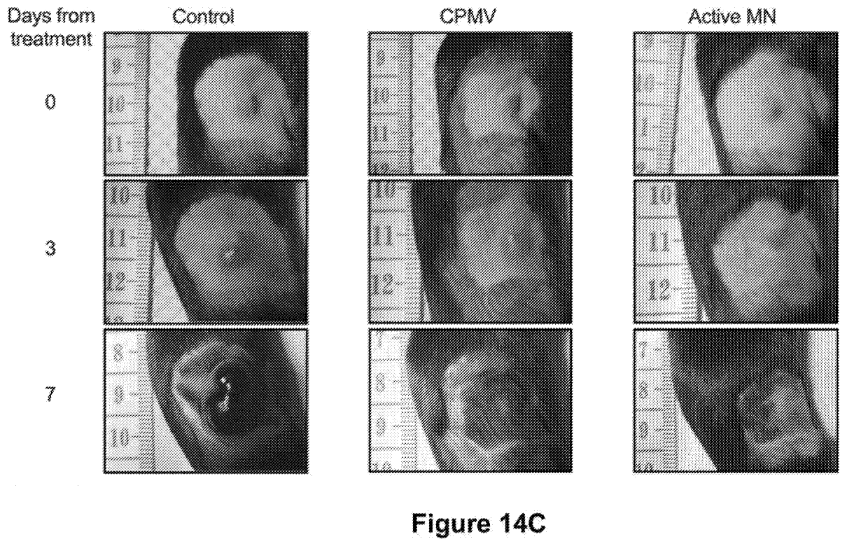

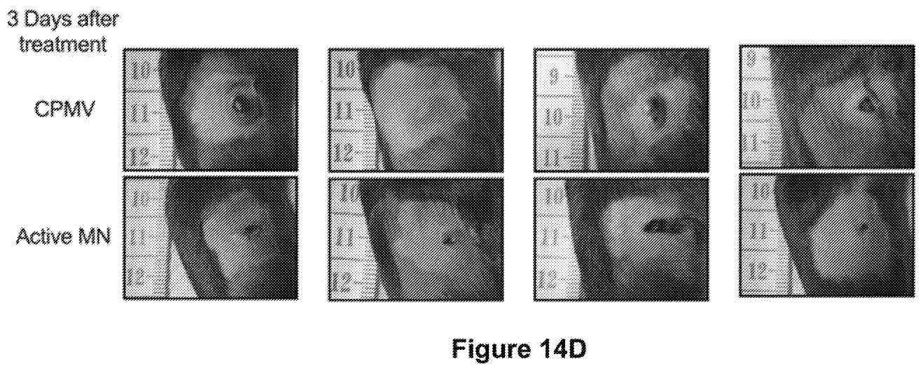

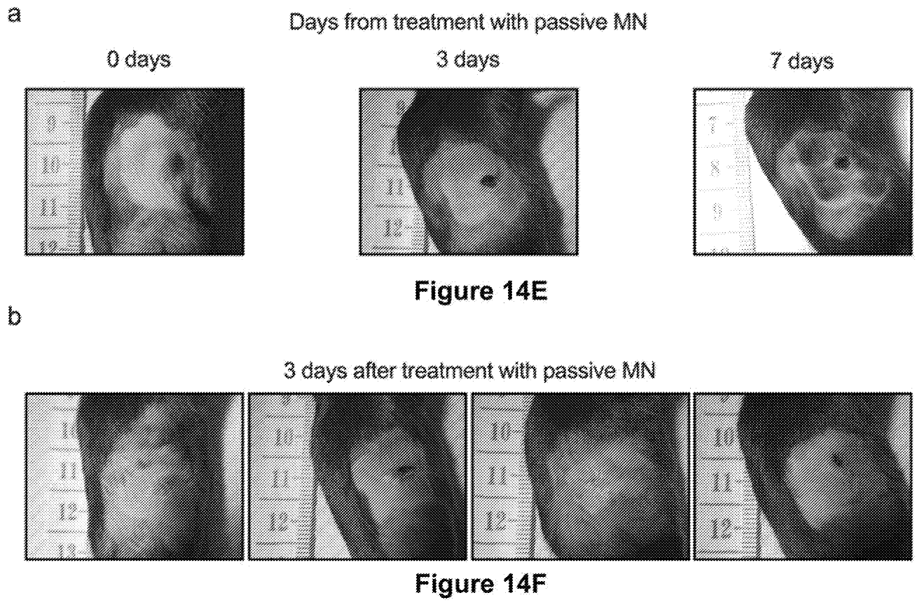

In some example implementations, the active MN patches can be used for CPMV in situ vaccination. In other example implementations, the active MN patches can be used for intertumoral in B16F10 dermal melanoma (see FIGS. 14A-14F). Initial investigations were aimed at determining the differences in IT delivery of CPMV via conventional needle injection and passive and active transdermal MNs in vivo. Adequate IT distribution and permeation of an immunoadjuvant in large or irregularly shaped tumors with conventional injection can be challenging, requiring multiple injections (e.g., 2 to 4 injections administered weekly) to induce a durable antitumor response and therapeutic efficacy. Dermal melanomas were produced by intradermal injection of B16F10 tumor cells in the right flank of C57BL/6 mice. When tumors reached approximately 40-80 mm³ in volume, 100 μg CPMV-loaded passive or active MNs were applied to the cutaneous surface of tumors until the needles completely dissolved (e.g., for 5-15 minutes).

FIGS. 14A-14F show CPMV in situ vaccination administered by an example embodiment of an active microneedle (MN) patches, passive MN patches or intratumoral injection in B16F10 dermal melanoma model. FIG. 14A shows images depicting an example embodiment of microneedle patches cut into smaller pieces (total of 4 or 9 pieces equaling full 100 μg CPMV dose) to cover tumor area. FIG. 14B shows a graph depicting the tumor volumes of mice receiving 30 μL PBS injection (PBS), 100 μg in 30 μL CPMV injection (CPMV), CPMV passive microneedle (MN), and CPMV active MN after treatment administration (on day 7 after intradermal B16F10 melanoma cell inoculation) (data are mean standard deviation (SD), n=5 for all treatment groups except for active MN group, n=6). Tumor growth was compared on different time points by one-way ANOVA with Tukey's test: *P<0.05, **P<0.01, ***P<0.001. FIG. 14C-14F shows a panel of images depicting the clinical appearance of representative PBS-, CPMV injection-, and active MN-treated tumors 3 and 7 days after treatment. FIG. 14D shows images at day 3 post treatment, CPMV injection-treated tumors vary between exophytic and involuted appearance. Active MN treated-tumors more consistently have an involuted appearance.

To facilitate patch placement on exophytic or irregular shaped larger tumors, the patches were cut to smaller pieces (e.g., 4-9 pieces) such that a full dose was administered over the contours of the mass with greater coverage of the tumor (see FIG. 14A). FIG. 14B shows the volumes of mice receiving 30 μL PBS injection (data labeled 14A), 100 μg in 30 μL CPMV injection (data labeled 14B), CPMV passive microneedle (data labeled 41C), and CPMV active MN (data labeled 14D) after treatment administration (on day 7 after intradermal B16F10 melanoma cell inoculation). The control mic for this experiment were intratumorally injected with either PBS (30 μL) or CPMV (100 μg/30 μL PBS). All animals received a single treatment administration.

In some implementations, the CPMV is potent when administered intratumorally via injection or MN patches. Greater tumor regression was observed in the passive and active MN-treated groups compared to injected CPMV-treated groups within the first three days post treatment. Injected CPMV did not lead to tumor regression in this time period, but rather appeared to slow progression relative to the PBS injection. Over the next 7 days, tumor progression was delayed the most in the active MN group, and progression was observed earlier in the CPMV and passive MN groups (see FIGS. 14B-14F). Furthermore, treatment with active MN patches appears to result in a more consistent clinical appearance of tumors. Physical examination of tumors 3 days after treatment, demonstrated that all melanomas treated with active MN and passive MN patches had an involuted appearance and were flattened on palpation (FIGS. 14C and 14F). In contrast, the appearance of tumors treated with CPMV injection varied from involuted to exophytic and on palpation varied from firm, rounded masses to a flattened center encircled by a rim of firm tissue (FIG. 14D). These tumor changes observed within 7 days of treatment have previously been related to changes in the TME and infiltrating innate immune cells responding directly to the presence of CPMV within the tumor. Hence, these findings suggest more uniformity in the delivery of CPMV with the active MN patches than with conventional injection, leading to consistent innate immune system-mediated antitumor effects.

In some example implementations, the active MNs exhibit an overall improvement in efficacy with a single treatment as compared to conventional techniques. When B16F10 melanomas reached approximately 25-30 mm³ in volume, 100 μg CPMV-loaded passive or active MN patches were applied to the tumors. Control mice were intratumorally injected with either PBS (30 μL) or CPMV (100 μg/30 μL PBS). The tumors were small and flat, permitting treatment without cutting the MN patches.

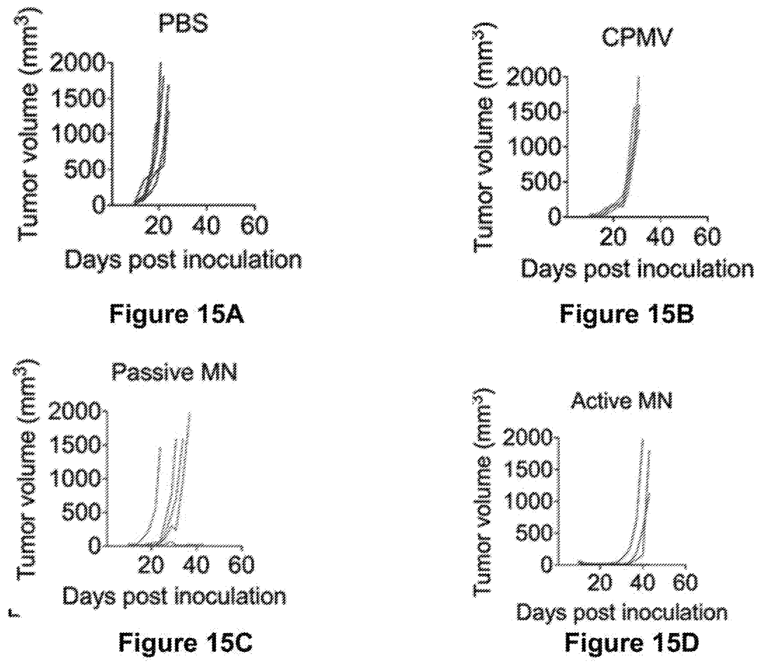

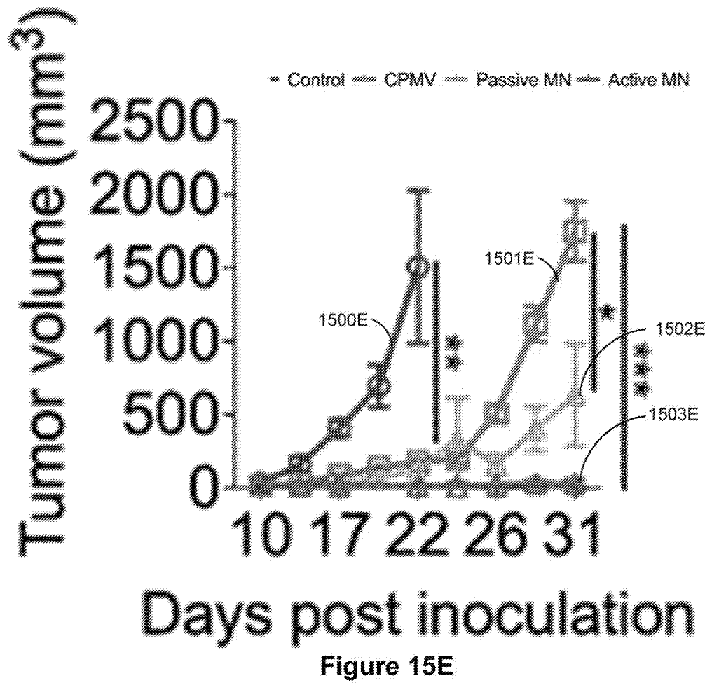

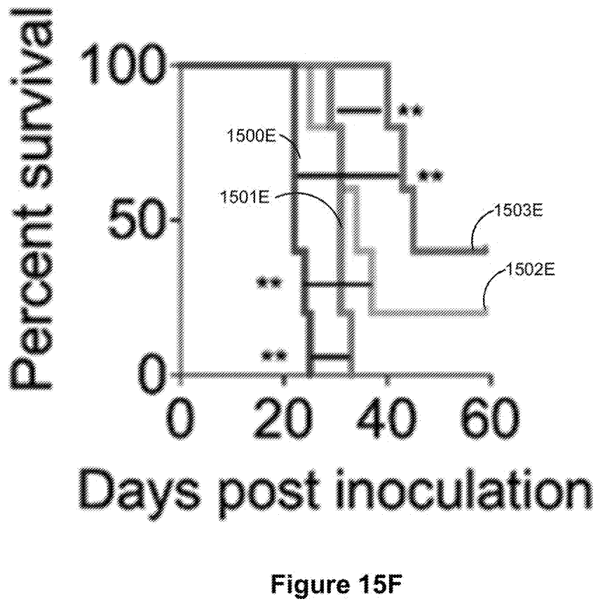

FIGS. 15A-15F show tumor growth suppression and survival with CPMV in situ vaccination administered by example embodiments of active MN, passive MN, and intratumoral injection in B16F10 dermal melanoma model. FIG. 15A-15D show tumor volumes growth curves of individual mice. FIG. 15E-15F show averaged tumor volumes of mice receiving PBS, CPMV injection, CPMV passive MN, and CPMV active MN (data are means±SEM (n=5)). Tumor growth was compared on different time points by one-way ANOVA with Tukey's test: *P<0.05, **P<0.01, ***P<0.001. Survival rates. Statistical significance was calculated using Log-rank (Mantel-Cox) test: **P<0.01.

FIGS. 15A-15D show that the tumor growth rate was slowest over time for mice administered active microneedles embedded with CPMV and Mg particles. FIG. 15E shows the average tumor volume of mice receiving PBS (plot 1500E), receiving CPMV injection (plot 1501E), CPMV passive MN injection (plot 1502E), and lastly, CPMV active MN injection (plot 1503E). As shown by FIG. 15E, the CPMV active MN injection resulted in the lowest average tumor size. (see FIG. 15E, plot 1503). However, all mice treated with CPMV injection had appreciable tumor growth after day 26 and were euthanized by day 31. CPMV-loaded passive MN patches caused further delay in tumor progression compared to injected CPMV (Passive MN: 464.4 (68.5-1364), CPMV: 1826 (1328-2095), p<0.05 on day 31). Mice treated with CPMV-loaded active MN patches had substantial suppression of tumor growth relative to those treated with CPMV injection (Active MN: 14.5 (9.1-68.6), p<0.001 on day 31, FIG. 15E, plot 1503E). With respect to survival, passive MN patches slightly improved the overall survival of mice, without a significant difference in median survival compared to CPMV injection (injected CPMV: 31 days vs passive MN: 34 days, FIG. 15E, plot 1502E). Moreover, 40% of the active MN-treated mice demonstrated durable survival with complete tumor rejection and prolongation of median survival to 45 days (FIG. 15F). Administration of CPMV in active MN patches led to enhanced early tumor regression, delayed tumor progression, and increased overall survival after a single administration. Similar efficacy level has been observed for CPMV administered intratumorally via injection in other preclinical studies of mouse tumors, however, it requires multiple treatments.

### In Vivo and Ex Vivo Imaging of CPMV Administration by IT Needle Injection, Passive MN, and Active MN

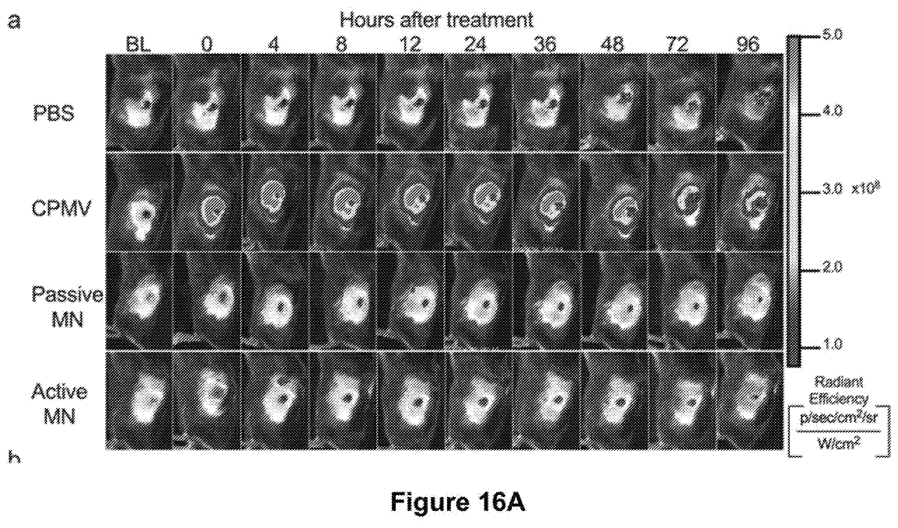

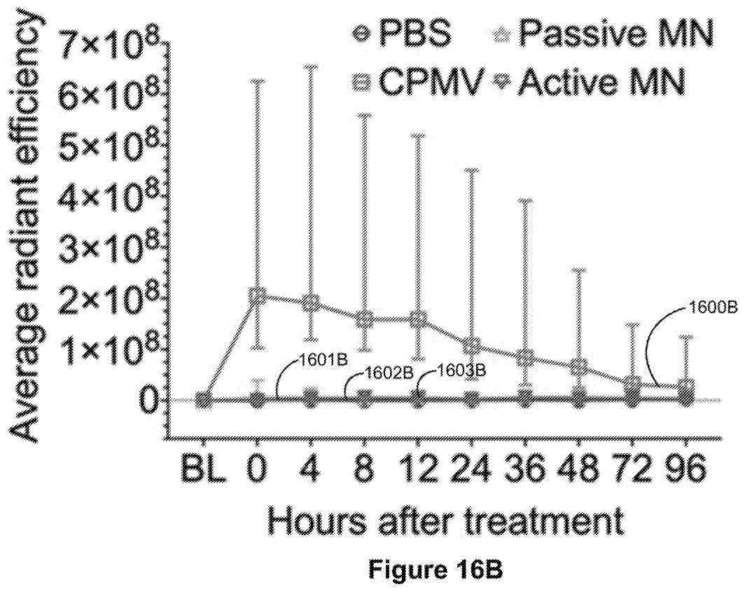

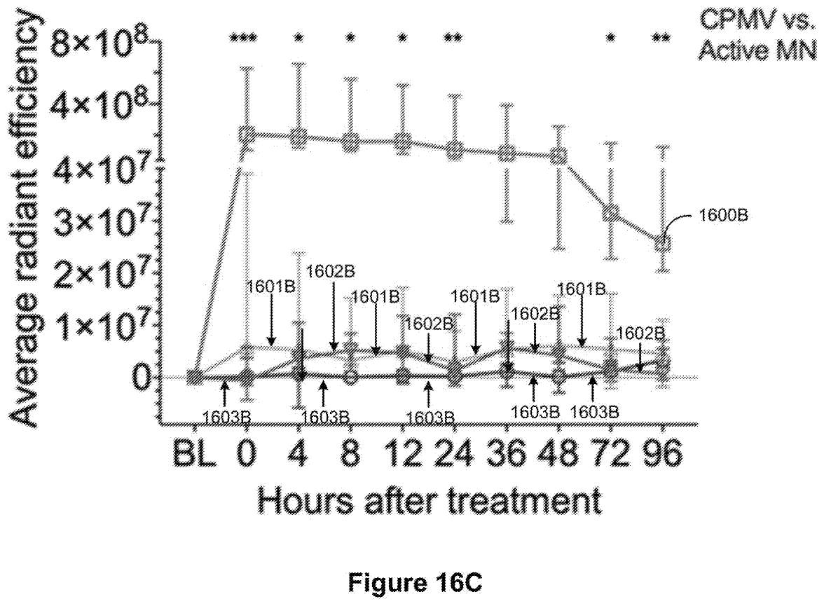

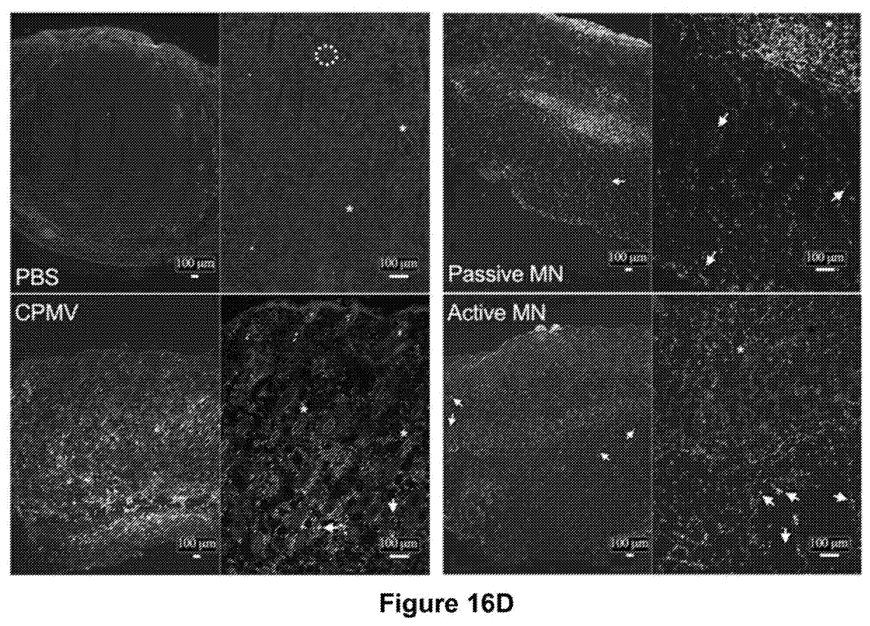

In some example implementations, after release from the active MNs, CPMV is widely distributed throughout the animal. Initial investigations involved determining that differences in distribution of CPMV released in vivo from active and passive MNs compared to IT injection may underlie differences in efficacy. Cy5-CPMV was employed to allow fluorescence imaging of CPMV nanoparticles (FIGS. 16-16D). Mice were inoculated with B16F10 melanoma isografts and Cy5-CPMV was administered to the resulting tumors (at volumes 60-100 mm³). For in vivo imaging, mice received 100 μg of intratumorally injected Cy5-CPMV, 100 μg of passive MN patch-administered Cy5-CPMV, or 100 μg of active MN patch-administered Cy5-CPMV. Tumors were imaged (IVIS Xenogen 200, Cy5.5 filter with excitation/emission range 615-665 nm/695-770 nm) serially to monitor CPMV release and retention at treatment site over time.

FIGS. 16A-16D show in vivo and ex vivo imaging of Cy5-conjugated CPMV (Cy5-CPMV) in situ vaccination of B16F10 melanomas administered example embodiments of active MN, passive MN, and intratumoral injection. FIG. 16A shows representative time course of in vivo fluorescence images of B16F10 melanomas treated with PBS injection, Cy5-CPMV injection, Cy5-CPMV passive MN, or Cy5-CPMV active MN. Colors denote radiant efficiency ((p/sec/cm²/sr)/(μW/cm²)) of Cy5-CPMV fluorescence. FIGS. 16B-16B show graphs depicting the quantification (average radiant efficiency) of Cy5-CPMV fluorescence in tumor ROI in vivo at different timepoints after treatment with PBS (n=4), Cy5-CPMV injection (n=5), Cy5-CPMV passive MN (n=7), or Cy5-CPMV active MN (n=7). Data are medians ±interquartile range. No significant differences in radiant efficiency was observed in Active MN vs. Passive MN and CPMV vs. Passive MN at any timepoints. Tumor growth was compared on different time points by Kruskal-Wallis one-way ANOVA with Dunn's multiple comparisons correction: *P<0.05, **P<0.01, ***P<0.001. FIG. 16C shows a panel of images showing the immunofluorescence of tumors 24 hours after treatment (blue: nucleus, pink: blood vessels, green: leukocytes, yellow: Cy5-CPMV, arrow: Cy5 CPMV, *: blood vessel, dashed circle: leukocyte). Abbreviations: BL, baseline; p, photon; sr, steradian; W, watt).

Injection of CPMV resulted in high levels of fluorescence at the tumor site that gradually decayed over time. FIG. 16A shows a representative time course of in vivo fluorescence imaging of B16F10 melanomas treated with PBS injection, Cy5-CPMV injection, Cy5-CPMV passive MN, or Cy5-CPMV active MN. The contrast in the images denote radiant efficiency ((p/sec/cm²/sr)/(μW/cm²)) of Cy5-CPMV fluorescence. As demonstrated in representative images (FIG. 16A), the fluorescence signal was not evenly dispersed throughout the tumor, but rather with peaks of fluorescence within discrete regions of the tumor. This suggested that CPMV was accumulating in various regions of the tumor, consistent with the heterogeneous structure of tumors and physical barriers with in the TME. Densely packed tumor cells and extracellular matrix structures may slow the rate of, or even impede passive diffusion of CPMV throughout the tumor. Studies of conventional needle-based injections demonstrate the initial production of discrete “depots” of the injected drug within the tissues, the shape and subsequent spread of which is influenced by the structures within the tissue itself. The injection technique could have produced depots of Cy5-CPMV within the tumor, which collectively may create a bright, but nonuniform fluorescent signal over the tumor.

For the active- and passive-MN treated tumors, the overall fluorescent signal detected was low and not significantly different from each other, nor the PBS group. The early tumor regression was still observed in these animals suggesting that the enclosed Cy5-CPMV was in fact released within these tumors. Some animals showed transient increase in signal in regions just inferior to the tumors, related to the tissue under the tumor into which the MNs were applied (FIG. 16A). The low fluorescence signal could result from the 100 μg Cy5-CPMV dose being dispersed over the area of the array of MNs. Prior studies of release from the active and passive MNs demonstrated initial formation of an array of smaller depots of payload drug, of roughly the size and shape of the MN. Over the course of 15 minutes, the payload diffuses away from the MN depot in a less concentrated, more uniform distribution over the area treated by MN array. This occurred more rapidly for payload released by active MNs than for that released by passive MNs. The Cy5-CPMV signal of the more thinly dispersed particles is likely to be relatively low, and may have been below the detection limit of the imaging system. Whereas for the conventional needle injections of Cy5-CPMV, the collective Cy5-CPMV particles in the depots could have led to a brighter fluorescent signal.

To further visualize the IT distribution of Cy5-CPMV, ex vivo immunofluorescence for employed of the in vivo treated tumors. B16F10 melanomas were generated and treated, as described above. Animals were euthanized and tumors resected en bloc and flash frozen in OCT at 24 h post treatment. Tumor sections were stained for blood vessels (CD31/PECAM-1), leukocytes (CD45), and cell nuclei (DAPI). Immunofluorescence of PBS treated tumor showed a vascular tumor with rare leukocytes. The Cy5-CPMV injection-treated tumor demonstrated uneven distribution of CPMV, with areas of greater Cy5-CPMV clustering seen as bright yellow regions at low power and high power (4× and 10× magnification, respectively). These brighter fluorescent areas of clustered Cy5-CPMV could be the appearance of the depots 24 h after injection. For the active and passive MN-treated tumors, Cy5-CPMV was observed to be less clustered within the tissues, bright yellow regions are not visible at low power. While at high power, discrete yellow puncta of Cy5-CPMV were observed, as compared to the larger, brighter clusters of Cy5-CPMV observed with conventional injection. Reduced clustering of Cy5-CPMV nanoparticles administered via MNs, would be consistent with the low fluorescence signal detected in the in vivo fluorescence microscopy images.

FIGS. 16B-16C show that widespread CD45 staining was also observed, indicating leukocyte infiltration of Cy5-CPMV treated tumors. FIGS. 16B-16C show quantification (average radiant efficiency) of Cy5-CPMV fluorescence in tumor ROI in vivo at different timepoints after treatment with PBS (plot 1603B, n=4), Cy5-CPMV injection (plot 1600B, n=5), Cy5-CPMV passive MN (plot 1601B, n=7), or Cy5-CPMV active MN (plot 1602B, n=7) where the data are medians ±interquartile range. No significant differences in radiant efficiency was observed in Active MN vs. Passive MN and CPMV vs. Passive MN at any timepoints. Lastly, FIG. 16D shows the immunofluorescence of tumors 24 hours after treatment, this data suggests that these early infiltrating immune cells are critical mediators of in situ vaccination efficacy.

### In Vivo Infiltration of Activated Antigen Presenting Cells and Tumoricidal Innate Immune Cells

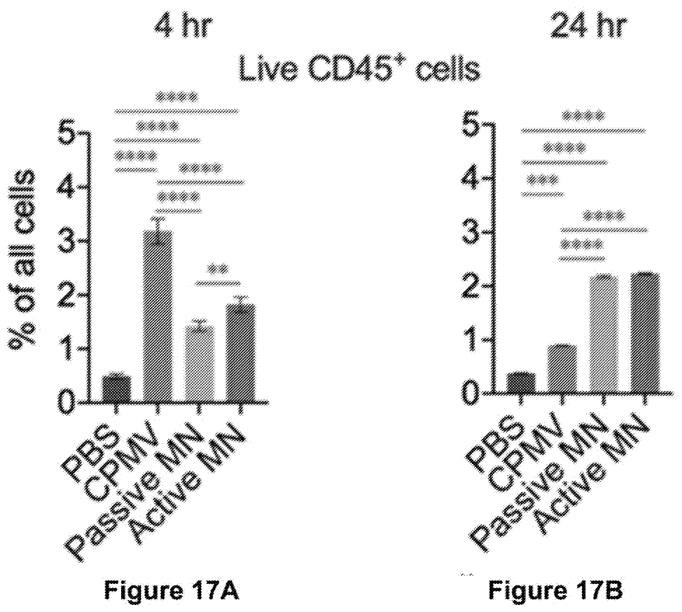

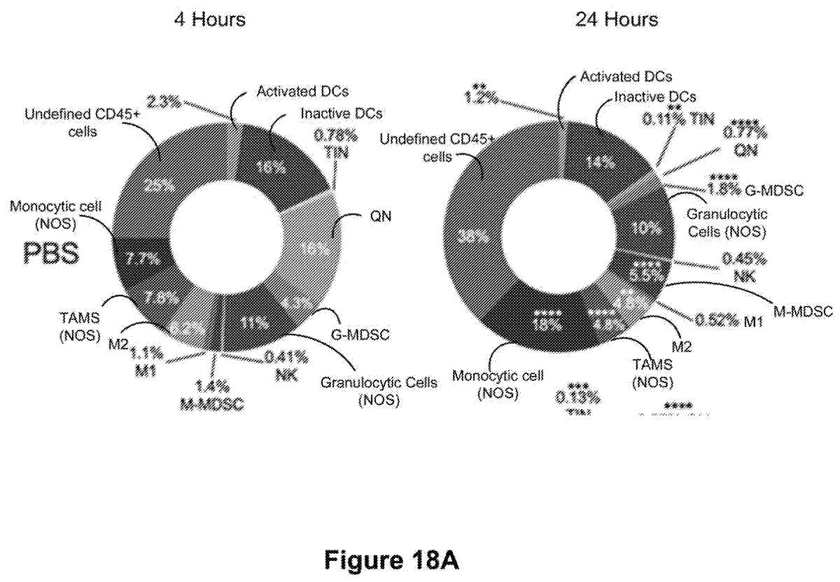

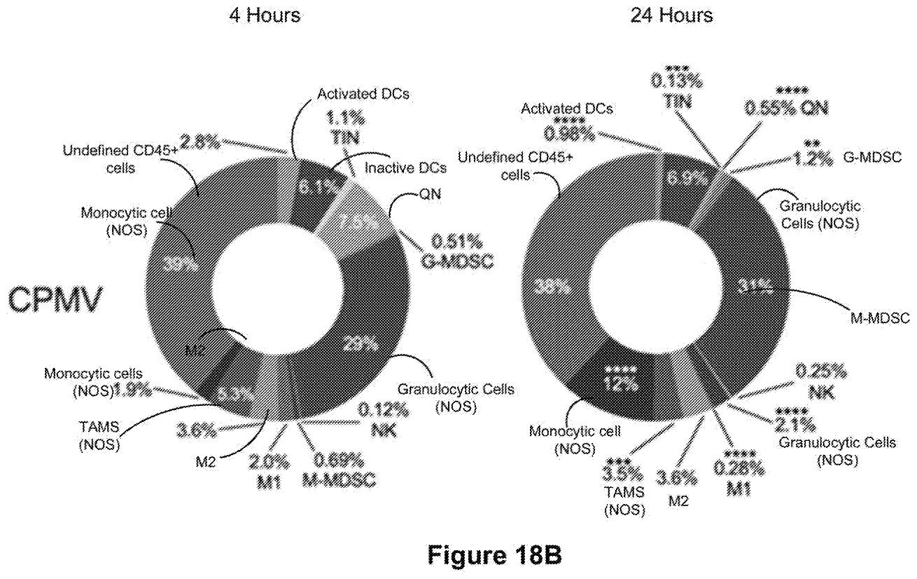

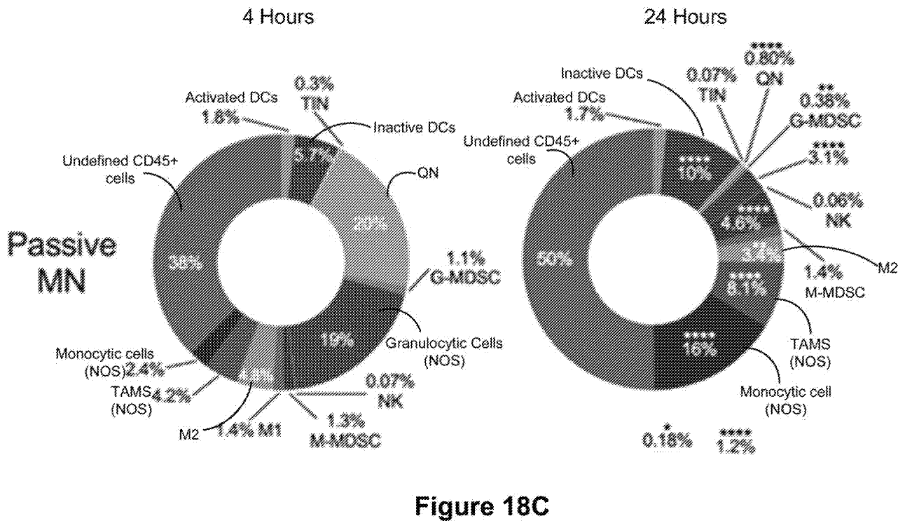

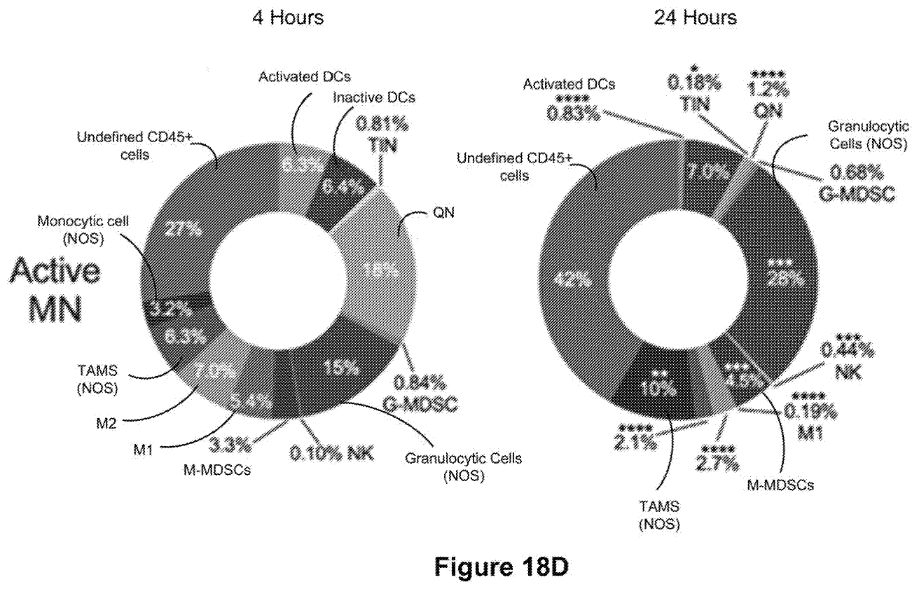

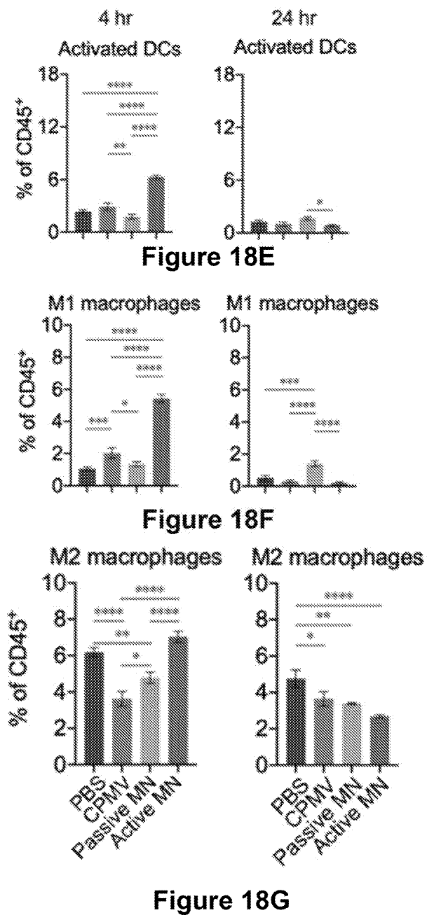

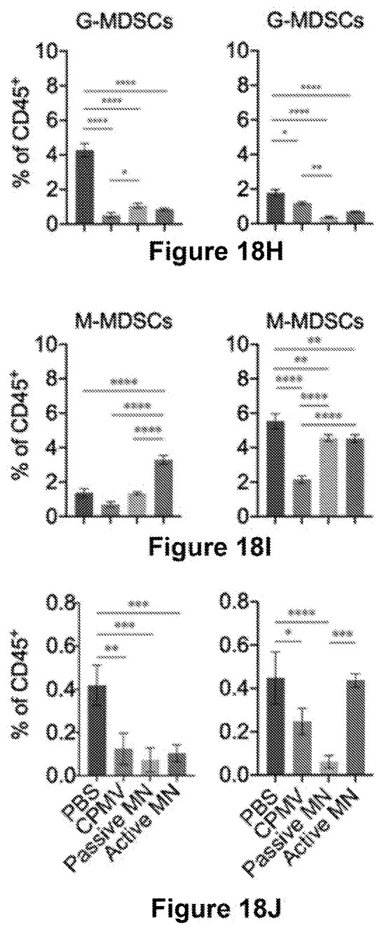

In some example implementations, the active MNs have an early cellular innate immune response. To investigate the early cellular innate immune response within the TME and its kinetics after treatment, the cellular immune infiltration of treated tumors was analyzed by flow cytometry at 4 and 24 h after either IT injection, passive MN, or active MN patch application (FIGS. 17A-17B and 18A-18J).

TABLE 1
Markers Investigated for Early Cellular Innate
Immune Response of the Active MNs

| Cell Types | Full Name | Phenotype |
|---|---|---|
| CD45 | Leukocytes | CD45⁺ |
| NOS | Not otherwise | CD45⁺CD11b⁺Ly6G⁻ |
| monocytic | specified | |
| cells | monocytic | |
| | cells | |
| NOS | Not otherwise | CD45⁺CD11b⁺Ly6G⁺ |
| granulocytic | specified | |
| cells | granulocytic | |
| | cells | |
| G-MDSCs | Granulocytic | CD45⁺CD11b⁺Ly6G⁺Ly6C⁻ |
| | myeloid- | MHCII⁻CD86⁻ |
| | derived | |
| | suppressive | |
| | cells | |
| M-MDSCs | Monocytic | CD45⁺CD11b⁺Ly6G⁻Ly6C⁺ |
| | myeloid- | MHCII⁻SSClow |
| | derived | |
| | suppressive | |
| | cells | |
| TINs | Tumor- | CD45⁺CD11b⁺Ly6G⁺MHCII⁺CD86⁺ |
| | infiltrating | |
| | neutrophils | |
| QNs | Quiescent | CD45⁺CD11b⁻Ly6G⁺ |
| | neutrophils | |
| Activated | Activated | CD45⁺CD11b⁺CD11c⁺MHCII⁺CD86⁺ |
| DCs | dendritic | |
| | cells | |
| Inactive DCs | Inactive | CD45⁺CD11b⁺CD11c⁺MHCII⁻CD86⁺, |
| | dendritic | CD45⁺CD11b⁺CD11c⁺MHCII⁺CD86⁻, or |
| | cells | CD45⁺CD11b⁺CD11c⁺MHCII⁻CD86⁻ |
| NK cells | Natural | CD45⁺CD11b⁺NK1.1⁺Ly6G⁻ |
| | killer cells | Ly6C⁻F4/80⁻ |
| M1 | Type 1 tumor- | CD45⁺CD11b⁺F4/80⁺Ly6G⁻ |
| macrophages | associated | Ly6C⁻MHCII⁺CD86⁺ |
| | macrophages | |
| M2 | Type 2 tumor- | CD45⁺CD11b⁺F4/80⁺Ly6G⁻ |
| macrophages | associated | Ly6C⁻MHCII⁻CD86⁻ |
| | macrophages | |
| NOS TAMs | Not otherwise | CD45⁺CD11b⁺F4/80⁺Ly6G⁻ |
| | specified | Ly6C⁻MHCII⁺CD86⁻, or |
| | tumor- | CD45⁺CD11b⁺F4/80⁺Ly6G⁻ |
| | associated | Ly6C⁻MHCII⁻CD86⁺ |
| | macrophages | |

FIGS. 17A-17B and 18A-18J show the intratumoral innate immune cell profile following administration of an example embodiment of a CPMV microneedle. C57BL/6 mice bearing dermal B16F10 tumors (60 mm³) were treated with CPMV by intratumoral injection, passive MN, or active MN. Four hours and 24 hours following treatment, the tumors were harvested to quantify innate immune cell infiltration by flow cytometry. FIGS. 17A-17B are graphs showing the percentage of total cells analyzed that were CD45+ at 4 hours (left) and 24 hours (right) after treatment with an example embodiment of a CPMV microneedle. FIGS. 18A-18D show pie graphs depicting the innate immune tumor infiltrate profiles (% of CD45+ cells) at 4 hours (left column) and 24 hours (right column) after treatment with an example embodiment of a CPMV microneedle. Data are mean percent (n=3). FIGS. 18E-18J show the percentages of intratumoral CD45+ cells consisting of activated dendritic cells (DCs, FIG. 18E), type 1 tumor associated macrophages (M1 macrophages, FIG. 18F), type 2 tumor associated macrophages (M2 macrophages, FIG. 18G), granulocytic myeloid-derived suppressive cells (G-MDSCs, FIG. 18H), monocytic-myeloid derived suppressive cells (M-MDSCs, FIG. 18I), and natural killer cells (NK cells, FIG. 18J). Data are means±SD (n=3). Statistical significance was calculated using two-way ANOVA (Treatment type vs. Time) with Sidak's multiple comparisons post-test: *P<0.05, **P<0.01, ***P<0.001, ****P<0.0001.

As shown in FIGS. 17A-17B, at 4 h following treatment, B16F10 melanomas treated with CPMV regardless of delivery method demonstrated increases in the percentage of IT CD45+ cells relative to that of PBS-treated tumors. The percent increase in tumors treated by CPMV injection was largest at 4 h, but only transient. By 24 h after treatment, the percentage of CD45+ cells in the CPMV injection-treated tumors had decreased below that of active MN- and passive MN-treated tumors, but was still significantly increased relative to the PBS-treated tumors. Active MN- and passive MN-treated tumors demonstrated a steady elevation in percentage of CD45+ cells at 4 h and 24 h relative to PBS-treated tumors (FIGS. 17A-17B).

Examination of the subpopulations of CD45+ cells demonstrated distinctive changes in the IT CD45+ immune cell profile at 4 h and 24 h after treatment with different delivery systems (FIGS. 18A-18D). As shown in FIGS. 18A-18D, activated dendritic cells (DCs, CD11b+CD11c+MHCII+CD86+) comprised a greater percentage of the CD45+ cell population in active MN-treated tumors than that of any other treatment group at 4 h.

FIGS. 18E-18J show that the passive MN patches and CPMV injection-treated tumors had similar percentages of activated DCs as the PBS injection-treated tumors (FIG. 18E). Notably, the percentage of activated DCs decreased more than 6-fold by 24 h after treatment in the active MN-treated tumors. Between 4 h and 24 h after treatment, the activated DC percentages decreased in the PBS-treated (1.9-fold) and CPMV injection-treated (2.9-fold) tumors (FIG. 18E, right panel). Activated DCs play a critical role in transporting antigens towards draining lymph nodes and lymphoid organs, as well as antigen presentation to prime subsequent adaptive immune responses. The active MN-treated tumors appeared to have greater recruitment of activated DCs at 4 h after treatment. The decrease at 24 h could represent the migration of these DCs to draining lymph nodes to present collected tumor antigens. Studies of DC migration from skin have demonstrated migration occurring over 1-3 days following immunogenic stimulation of the skin.

The monocytic component (CD11b+ Ly6G−) of the CD45+ cell population exhibited complex, dynamic changes after the different methods of CPMV administration. The monocytic component included Type 1 tumor-associated macrophages (M1s, CD11b+F4/80+Ly6G−Ly6C−MHCII+CD86+), Type 2 tumor-associated macrophages (M2s, CD11b+F4/80+Ly6G−Ly6C−MHCII−CD86−), natural killer cells (NK cells, CD11b+NK1.1+Ly6G−Ly6C− F4/80−), monocytic-myeloid derived suppressive cells (M-MDSCs, CD11b+Ly6G−Ly6C+MHCII−SSClow), tumor-associated macrophages, whose phenotype was not further specified (TAMs not otherwise specified (NOS), CD11b+F4/80+Ly6G−Ly6C−MHCII+CD86− and MHCII−CD86+), and monocytic cells NOS (CD11b+Ly6G−). This NOS designation refers to the remainder of cells within a lower level gate that were not found in higher level gates with the flow cytometry gating strategy. For example, monocytic cells NOS refers to a population of cells that were found to be CD11b+Ly6G−, but did not fall into higher level gates where they would have been designated as M1, M2, TAMs NOS, NK, or M-MDSCs. TAMs NOS are the remainder of CD11b+F4/80+Ly6G−Ly6C− cells that were not also in the higher level MHCII+CD86+ or MHCII−CD86− gates, in which the cells were further specified as M1 or M2, respectively.

The immune response observed in active MN-treated tumors maintained a similar level of the monocytic component (CD11b+Ly6G−) of the CD45+ cell population relative to the PBS-treated tumors at 4 h post-treatment, while the levels in CPMV-injected and passive MN-treated tumors were relatively suppressed. Interestingly, while PBS-treated tumors and active MN-treated tumors had similar percentages of monocytic cells comprising the CD45+ population, within this component, the active MN-treated tumors had greater percentages of M1s and M-MDSCs than the PBS-treated tumors. The M1 percentage of active MN-treated tumors was also significantly greater than that of CPMV injection-treated or passive MN-treated tumors (FIGS. 18A-18D, 18D, and 18I, left panels). The percentages of M2s were comparable between the PBS and active MN-treated tumors but were lower in the CPMV injection- and passive MN-treated tumors at 4 h (FIG. 18G, left panel). The NK cell percentage was suppressed at similar levels in all CPMV-treated tumors at 4 h, relative to the PBS-treated tumors (FIG. 18J, left panel). The early increase in the monocytic component of the immune response in the active MN-treated tumors seems to be driven by increases in the percentages of M1s, M-MDSCs, and monocytic cells NOS.

By the 24 h timepoint, the percentage of the monocytic component in PBS-injected tumors continued to increase. The monocytic component in the active MN-treated tumors decreased and CPMV injected-tumors increased until both reached comparable levels. The percentage in the passive MN-treated tumors increased to a similar level as that of the PBS-treated tumors (FIGS. 18A-18D, right panel). The M1 percentage decreased in all groups, except for the passive MN-treated group, which did not change from the 4 h level (FIG. 18H, right panel). The M2 percentage decreased, relative to PBS-treated tumors, in all CPMV-treated groups at 24 h (FIG. 18G, right panel). The percentage of M-MDSCs increased in all groups at 24 h after treatment, with the PBS-treated group having the largest percentage of M-MDSCs and the CPMV injection-treated group having the lowest M-MDSC percentage of all groups (FIG. 18I, right panel). The tumors treated with active MNs recovered the NK cell percentage to the level of the PBS-treated tumors at 24 h, while this level remained suppressed among the CPMV injection- and passive MN-treated tumors (FIG. 18J, right panel).

The monocytic component consists of a mix of cells, including macrophages, monocytes, and M-MDSCs. Macrophages can exist in different functional states depending upon their surrounding environment. M1 macrophages promote a pro-inflammatory state and have antitumor activity. M2 macrophages promote tumor growth and progression. Several, sometimes conflicting, roles have been attributed to M-MDSCs. They are immunosuppressive and support tumor progression. However, there is evidence that they can also differentiate into different types of macrophages and DCs. The fate of these cells is at least partially influenced by the state of the surrounding TME. The early increase in M-MDSC percentage in the active MN-treated tumor could represent an increased pool of potential macrophages and DCs, and would be consistent with the increased percentage of active DCs and M1s, as well as M2s. Although at 4 h, the PBS-treated tumors had similar levels of M2s as the active MN-treated tumors, they also contained a reduced percentage of M-MDSCs, so these could have arisen from a different source or developmental pathway in the PBS-treated mice. Further, the monocytic cells NOS component represents a heterogeneous mix of monocytic cells, which include monocytes that also can differentiate into M1s or M2s. Macrophages have long life spans and enhanced phagocytic capacity, especially compared to that of neutrophils. Macrophages have been implicated as critical mediators of tumor regression through direct tumoricidal activity. The increased percentages of differentiated M1s, potential macrophage progenitors, and greater recovery of NK cells in active MN-treated tumors suggest a greater portion of the CD45+ infiltrate may be comprised of cells with pronounced ability to destroy tumor cells. This could underlie the augmented tumor regression observed in the tumors treated with active MNs. Passive MN-treated tumors appeared to have a delayed expansion of the monocytic cells NOS and M-MDSCs, but not M1 macrophages, at the 24 h timepoint. CPMV injection-treated tumors exhibited a more modest increase in its M1 percentage and NK cell recovery. Both CPMV injection and passive MN administration demonstrated inferior suppression of tumor growth.

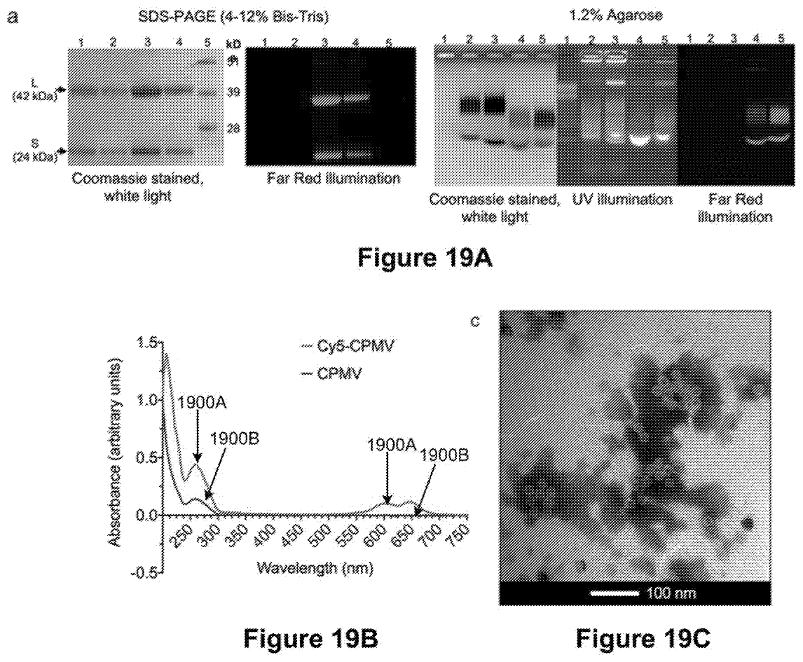

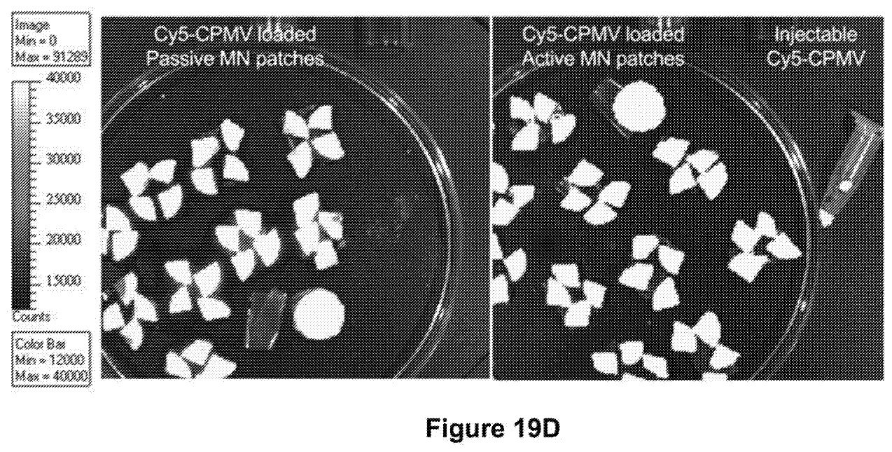

With respect to granulocytic cell (CD11b+Ly6G+) and quiescent neutrophil (QN, CD45+CD11b−Ly6G+) components of the CD45+ infiltrate, dynamic changes in these subsets were also observed. The percentage of broadly granulocytic cells within CPMV injection-treated tumors expanded, compared to that of the PBS-treated tumors, and remained consistent at 4 h and 24 h after treatment (FIG. 18A-18D). These cells included tumor infiltrating neutrophils (TINs, CD11b+Ly6G+MHCII+CD86+), granulocytic myeloid-derived suppressive cells (G-MDSCs, CD11b+Ly6G+Ly6C−MHCII−CD86), and those whose phenotype was not further specified (granulocytic cells not otherwise specified (NOS), CD11b+Ly6G+ cells that were not designated as TINs or G-MDSCs) (FIGS. 19A-19D).

FIGS. 19A-19D show an example embodiment of Cy5-CPMV loaded active and passive MN patch characterization. FIG. 19A shows a gel analysis of CPMV and Cy5-CPMV. denaturing (SDS-PAGE, left) gel: 1: CPMV (10 μg), 2: CPMV (5 μg), 3: Cy5-CPMV (10 μg), 4: Cy5-CPMV (5 μg), Lane 5: ladder. Native (1.2% agarose, right) gel: 1: 1 kb DNA ladder, 2: CPMV (5 μg), 3: CPMV (10 μg), 4: Cy5-CPMV (5 μg), 5: Cy5-CPMV (10 μg). FIG. 19B shows a UV-visual spectral analysis of CPMV and Cy5-CPMV, with absorbance peak at 647 nm in Cy5-CPMV. FIG. 19C shows a transmission electron microscopy of Cy5-CPMV (0.1 mg/ml). FIG. 19 D shows fluorescence images of example embodiments of Cy5-CPMV loaded active MN, passive MN, and injectate.

TIN percentage did not differ from PBS-treated tumor levels in any groups at 4 h and 24 h after treatment (FIG. 19B). FIG. 19B show as an absorbance of Cy5-CMPV and CPMV and includes a plot 1900A representing Cy5-CPMV and a plot 1900B representing CPMV. The quiescent neutrophil (QN, CD45+CD11b−Ly6G+) percentage was suppressed in CPMV injection-treated tumors relative to PBS, passive MN- and active MN-treated tumors at 4 h as demonstrated in the TEM of Cy5-CPMB (0.1 mg/ml) in FIG. 19C. FIG. 19D shows fluorescent images of Cy50CPMV loaded passive MN patches (left panel) and the Cy5-CPMB loaded active MN patches (right panel).

The granulocytic cell percentage in active MN-treated tumors increased to a level comparable to that in the CPMV injection-treated tumors by 24 h, while the granulocytic component decreased in the passive MN-treated tumors (FIGS. 18A-18D, right panel). This overall increase within the active MN-treated tumors was related to the higher percentage of granulocytic cells NOS within the tumors, while the G-MDSC percentage was decreased in all CPMV-treated tumors, relative to PBS-treated tumors at 4 h and 24 h after treatment (FIGS. 18A-18D and FIG. 18F). The percentage of quiescent neutrophils decreased to a similar level in all groups by 24 h (FIGS. 18A-18D). CPMV injection-treated tumors exhibited a more pronounced increase of the granulocytic cell NOS component. Granulocytic cells NOS is also a heterogenous mix of cell types, including activated neutrophils. Previous studies have demonstrated that injection of CPMV for in situ vaccination led to increases in the percentages of activated neutrophils and TINs, with suppression of QNs within the first 24 h of treatment. Thus, the observed increase in the CPMV injection-treated tumors is consistent with previous reports. The slower increase in the granulocytic component of the immune cell response over 24 h, with a greater percentage of monocytic cells, notably NK cells and M1s, which possess well-known tumoricidal functionality, following active MN treatment represents a difference from injection.

It is believed that this increased efficacy and immune responses using the active MN-based CPMV administration is related to the differences in the kinetics of CPMV delivery. It further contemplated that increased efficacy is a result of the potential actions of the Mg micromotors themselves. Mg²⁺ is an important second messenger and has been implicated in T cell stimulation in response to antigens, macrophage development, M1/M2 polarization, and DC migration. These in vitro studies examined substantially higher extracellular Mg²⁺ concentrations (approximately 6 times greater) than the maximum possible concentration of Mg released from the active MN patch into the smallest volume (25 mm³) tumor in this study. While Mg²⁺ at higher concentrations plays an important role in T cell activation, it is unlikely that Mg²⁺ would reach distant lymphoid structures at concentrations relevant to CD8+ T cell activation in vivo. The relatively low Mg²⁺ concentration within the tumor and washout over time, would likely limit Mg²⁺ contribution to the overall enhancement of immune responses beyond small, short-lived effects. Moreover, in a previous report, active MN patches devoid of any payload therapeutic (blank MNs) did not demonstrate antitumor efficacy, as durable tumor growth suppression and overall survival in blank MN-treated mice did not differ significantly from those treated with PBS injection.

Overall, these results demonstrated that CPMV in situ vaccination via active MNs promoted enhanced IT recruitment and activation of APCs, including DCs and macrophages. The enhanced infiltration of macrophages is also consistent with the pronounced early tumor regression. Passive MNs, however, seemed to lack enhancement of activated DCs and M1s, with a delayed infiltration of other monocytic cells.

### Ex Vivo Cytotoxic CD8+T Lymphocyte Activity

In some example implementations, the active MNs lead to an enhanced antitumor adaptive immune response. In situ vaccination optimally results in induction of a systemic antitumor response mediated by the adaptive immune system. Local innate immune activation is critical for priming these adaptive immune responses. To determine whether the remodeling of the TME and rapid infiltration of APCs by active MN treatment could effectively launch and improve the systemic antitumor response of CD8+ T cells, an interferon-γ (IFN-γ) release assay with splenocytes from treated mice was performed. CD8+ T cells producing IFN-γ indicates activation of the cells in response to recognition of their target antigen. B16F10 melanomas were treated when volumes reached 60 mm³. Splenocytes were isolated 10 days after treatment with PBS, CPMV injection, passive MN, or active MN as described above. The splenocytes were incubated with B16F10 melanoma cell lysate, CPMV, or culture media only in a suspension culture for 48 h. After this period, IFN-γ-producing effector CD8+ T cells (CD44hiIFN-γ+CD8+) frequency was evaluated using flow cytometry.

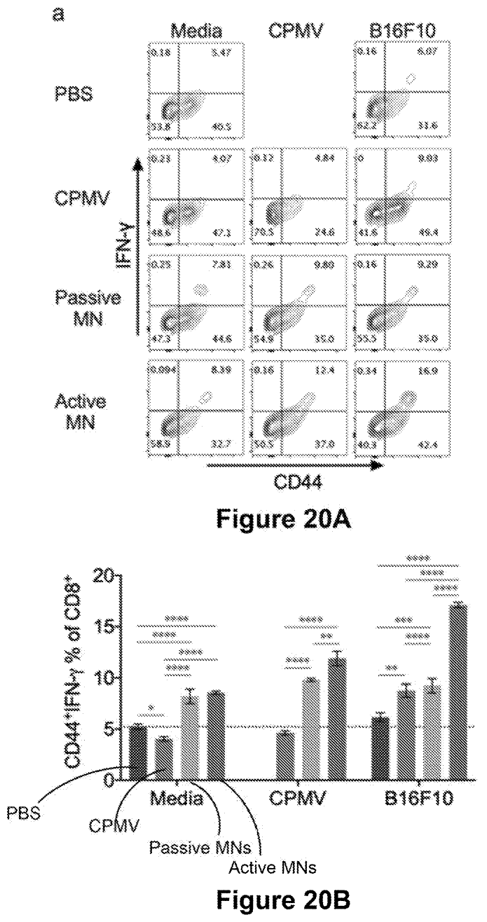

FIGS. 20A-20B show the systemic anti-tumor immune response following CPMV microneedle administration. C57BL/6 mice bearing dermal B16F10 tumors (60 mm³) were treated with CPMV by intratumoral injection, passive MN, or active MN 10 days after B16F10 cell inoculation. Ten days following the treatment, spleens were harvested and co-cultured with media, 10 μg CPMV, or B16F10 tumor cell lysate for 48 h. Intracellular IFN-γ was measured in CD8+ T cells by flow cytometry.

As shown in FIGS. 20A-20B, co-incubation of B16F10 melanoma cell lysate with splenocytes from mice treated with CPMV injection, passive MN, and active MN exhibited increased antigen-specific CTL activity. FIG. 20A is a representative flow cytometry plots of CD44^{hi}IFN-γ+CD8+ T cells in each re-stimulation group. FIG. 20B is a plot showing the percentage of CD44^{hi}IFN-γ+CD8+ T cells after gating CD8+ T cells (data are means±SD (n=3)). The dashed line indicates the background level activation. Splenocytes from PBS-treated animals were omitted from CPMV stimulation. Statistical significance was calculated using one-way ANOVA with Tukey's multiple comparisons post-test: **P<0.05, **P<0.01, ***P<0.001, ****P<0.0001).

FIGS. 20A-20B show that the active MN treatment group had the greatest percentage increase of IFN-γ-producing effector CD8+ T cell (p<0.0001 vs. other treatment groups) after B16F10 melanoma cell lysate exposure. Incubation of splenocytes from mice treated with CPMV via passive and active MNs with CPMV increased the frequency of activated effector CD8+ T cells by 2.11-fold (vs. PBS, p<0.0001) and 2.57-fold (vs. PBS, p<0.0001), respectively compared to CPMV injection-treated mice. Splenocytes from mice treated with CPMV injection incubated in media alone demonstrated no change in CD8+ T cell population compared to PBS-treated group. In contrast, a 1.5-fold increase of effector CD8+ T cell population was shown in both passive MN- and active MN-treated mice.

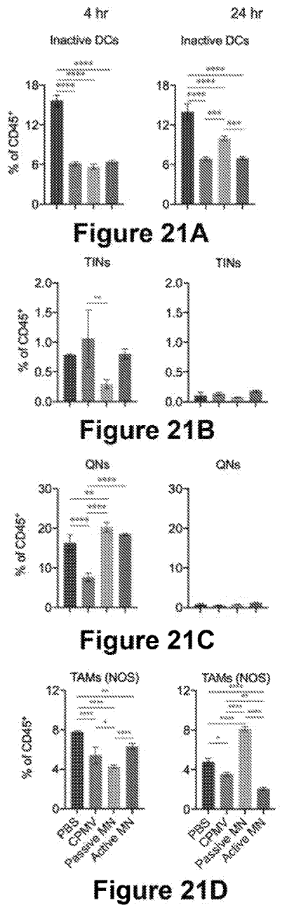

FIGS. 21A-21D show intratumoral innate immune cell profile following CPMV microneedle administration. Percentages of intratumoral CD45+ cells consisting of inactive dendritic cells (DCs, FIG. 12A), tumor-infiltrated neutrophils (TINs, FIG. 21B), quiescent neutrophils (QNs, FIG. 21C), and tumor-associated macrophages not otherwise specified (TAMs NOS, d) within the CD45+ leukocytes from samples harvested 4 h after treatment (left) and from samples harvested 24 h after treatment (right). Data are means±SD (n=3). Statistical significance was calculated using two-way ANOVA (Treatment type vs. Time) with Sidak's multiple comparisons post-test: *P<0.05, **P<0.01, ***P<0.001, ****P<0.0001. The data depicted in FIGS. 21A-21C further support the characterization provided in FIGS. 17A-17B and 18A-18J.

Larger percentage of CD8+ T cells producing IFN-γ is associated with enhanced antigen priming and presentation by APCs, as well as greater suppression of tumor growth in other cancer vaccination strategies. CPMV treatment with active MNs promotes activation of a larger percentage of the CD8+ splenocyte population by CPMV or the targeted tumor. Increased APC cross presentation of CPMV antigens and tumor antigens with active MN treatment may mediate the heightened CD8+ T cell activation. Potential broader distribution of CPMV within the heterogenous tumor, with active MN administration, may also lead to APC collection and cross presentation of a more diverse array of tumor antigens in the lymphoid organs. In turn, this may induce activation of a broader subset of antitumor CD8+ T cells This finding supports the previous hypothesis that rapid, augmented APC infiltration into the TME with active MN-mediated CPMV delivery (FIGS. 14C-14D), could lead to a more potent systemic antitumor response than that following CPMV injection. Treatment with active or passive MNs also led to an expansion in the percentage of activated CD8+ splenocytes, even in the absence of B16F10 cell or CPMV exposure. These cells could represent enrichment in the CD8+ T cell population recognizing antigens within the culture conditions alone. This suggests that the process of in situ vaccination with MNs may generally enhance CD8+ T cell activation.

In some embodiments, MNs containing Mg microparticles can be used to deliver nanoparticles, specifically the plant viral nanoparticles, CPMV, for in situ vaccination against the B16F10 model of melanoma. In some embodiments, the MNs provide rapid release of CPMV from the active MN platform in vitro. Administration of CPMV with active MNs can enhance tumor regression compared to conventional injection.

### EXAMPLE MATERIALS AND METHODS

### Example 1

In the example implementations described, fabrication of microneedles of different materials were performed. Microneedle arrays with a number of 225 tips had conical shape and presented dimensions of 1000 μm in height and 400 μm in base. Microneedles were made from polyvinylpyrrolidone with molecular weight of 360K. Microneedles were subjected to a variety of characterization methods, such as: imaging (Scanning Electron Microscopy), dissolution properties, and mechanical testing. Additionally, a micromolding process was performed to reproduce negative microneedle molds made from PDMS, and as well fabrication of phantom gel tissue samples.

### Materials

In some example implementations, polyvinylpyrrolidone (PVP) average Mw˜360,000, PDMS base curing agent kit SYLGARD® 184, and agarose where purchased from Sigma Aldrich. HRP-Goat anti-human IgG Antibody (peroxidase) from Vector Laboratories. Goat anti mouse IgG-Alexa Fluor 555 from Abcam. Magnesium microparticles with size >=45 m, catalog #FMW40, from TangShanWeiHao Magnesium Powder Co., Ltd China. 0.9 μm Nile red fluorescent particles from Sphero Tech. Porcine skin was obtained from a near market. 3D printing UV sensitive resin was obtained from AnyCubic.

### PDMS Microneedle Mold Fabrication

In some example implementations, the master microneedle mold was placed in a clean Petri Dish, Crystal Clear Borosillicate Glass with a double-sided tape to attach the mold properly. A mix ratio of 10:2 base/curing agent PDMS solution was later casted onto the microneedle patch and placed in vacuum within a desiccator for 5 min at 23 in of Hg. Bubbles were removed from the surface and PDMS was cured in an oven at 75 C for 30 min. Later sample was removed from the oven and cured PDMS was separated from petri dish gently to obtain the negative mold. PDMS mold was adjusted to desired size with the use of a blade cut.

Microneedle molds were washed with hand soap and rinsed with water twice, with further Ultrasonication bath for 15 min. Later, the mold was dried with air gun and cleaned by adding 0.25 mL of 2-propanol to each mold for 10 min. Molds were placed in the oven (75 C for 15 min) and not used until they reached room temperature.

### Microneedle Patch Fabrication

In some example implementations, a volume of 0.25 mL of polymer (PVP) was added onto the previously cleaned PDMS microneedle mold and further placed in a closed desiccator in vacuum for 5-10 min (23 in Hg). Molds were removed from desiccator with the further removal of bubbles generated at interface between microneedle pores and solution with the use of plastic 1 mL disposable transfer pipettes or the use of a tweezer. Later, bubbles in the surface of the solution were removed, or popped with the use of a pipette or needle tip. Furthermore, a second addition of 0.25 mL of polymer was carried out, turning on vacuum again. This process was performed until polymer solution was 1 mL.

The payload solution (50 μg of IgG-HRP, 50 μg of IgG-AlexaFluor555, 20 μg of Rhd6G, 20 μg of FITC, or 100 ug of anti-CTLA-4) was added to the mold and let it to dry for 24-72 hours. Once microneedle patches were ready, a 1 cm² scotch tape was applied on top of the needles and peeled off from the PDMS mold. Microneedle patches were stored at room temperature prior to use.

### Phantom Skin Mimicking Gel Fabrication

In some example implementations, 2% Agarose was weighted in a 20 mL Crystal Clear Borosilicate vial in DI water. Solution was heated a 175 C until solution turned transparent. Later, the temperature was lowered to 120 C and casted onto 1.5 mm, 3.0 mm or 4.5 mm Eco-Flex negative molds. Solution was let it dry for 2 min and further removed form mold with help of tweezers. Phantom skin mimicking gel were soaked in PBS pH 7.4 prior use.

### Scanning Electron Micrograph

In some example implementations, microneedle patches were characterized by Scanning Electron Microscopy in a FEI Quanta SEM System. The array was previously sputtered with an Iridium coating and placed on Quanta chamber to make it conductive. Microneedles were imaged at 2-5 KV spot n=3.

### Mechanical Testing

In some example implementations, a mechanical test was performed to polyvinylpyrrolidone microneedles by applying a constant load to a single tip. Microneedles show a force load capability (0.5 N/needle) by being good candidates for skin penetration. The mechanical strength of microneedles was measured by visualizing the displacement of the microneedle tip structure compared to the relative height of the cone vs the applied force with the use of a Force Gauge Model M4-20 system Mark0-10 Series 4.

### Release Kinetics

In some example implementations, microneedles were subjected to a test of piercing and further dissolved into a 3 mm thickness 2% Phantom Tissue Skin with a PBS pH 7.4 reservoir below. Both passive and active microneedles (n=5) were loaded with 50 μg of Rhodamine 6G or IgG-HRP. After piercing for different set times, the supernatant was collected and analyzed by a Shimatzu UV-vis spectrophotometer from 300 to 700 nm. For the electrochemical measurement of IgG-HRP, a reservoir with TMB+H₂O₂ was placed below the phantom tissue and repetitive runs of amperometry at a fixed potential of +0.1V for 50 s were employed to analyze the current change behavior of both microneedle controls.

### Skin Penetration and Diffusion Studies

In some example implementations, a 1.5 mm thickness porcine skin area of 2×2 cm was pierced by microneedles for both diffusion and active control studies. The microneedle patches were placed for different interval times, 5, 10, and 20 min and further cross sectioned for analysis at room temperature.

### Cell Line

In some example implementations, the B16F10 cell line was acquired from American Type Culture Collection (ATCC). B16F10 cells were cultured in Dulbecco's Modified Eagle's Medium (DMEM, Life Technologies), supplemented with 10% (v/v) fetal bovine serum (FBS, Atlanta Biologicals) and 1% (v/v) penicillin-streptomycin (Life Technologies). Cells were maintained at 37° C., 5% CO₂. The cell cultures were maintained below a 50% confluence and early passage culture were utilized for the experiments.

### In Vivo Efficacy Study in Mice

In some example implementations, the described in vivo efficacy experiments were conducted in accordance with UCSD's Institutional Animal Care and Use Committee. 6- to 8-week-old female C57BL/6 mice (The Jackson Laboratory) were used. 25,000 B16F10 cells were suspended in 50 μL PBS and were injected intradermally into the right flank of C57BL/6 mice on day 0. PBS, anti-CTLA4 antibody (100 μg, Clone, 9H10, BioXcell) were administrated into mice by intratumoral injection (30 μL) or by microneedle on day 10 and day 17. Tumor volumes were measured using a digital caliper. The tumor volume (mm³) was calculated as (long diameter×short diameter²)/2. Animals were sacrificed when tumor volume reached 1500 mm³.

### Stereolithography 3D Printed Microneedle Devices

In some example implementations, the prototyping of solid microneedles is currently supported by paid or free commercial software (SolidWorks, Fusion360). The 3D microneedle STL models were transferred to a slicing software (AnyCubic Photon slicer64), which sliced the 3d model into thousands of micron layers in a 30M file, later connected to the printer via USB.

The file was uploaded to an AnyCubic Photon UV LCD 3D printer for the prototyping and printing. Microneedles were fabricated within a 115×65 mm build plate, by using exposure times of 8 s and step size of 20 um. This instrument projects a 25 W UV light source that sits inside a stainless-steel snoot, through a photocurable material. The 3D printer has a 2K LCD masking screen. 2560*1440(2 k) HD masking LCD provides very fine printing details down to few micrometers.

After the fabrication, the build plate containing microneedle devices was gently removed from the printer, and microneedle devices were detached. Supports printed to build the microneedle devices were removed, microneedles were rinsed in IPA and placed under an ultrasonic bath for the removal excess of uncured material. Microneedle devices were subsequently placed in a UV nail machine to post cure for 30 min.

### Example 2

In the example implementations described, fabrication of microneedles of different materials were performed. Microneedle arrays with a number of 225 tips had conical shape and presented dimensions of 1000 μm in height and 400 μm in base. Microneedles were made from polyvinylpyrrolidone with molecular weight of 360K. Microneedles were subjected to a variety of characterization methods, such as: imaging (Scanning Electron Microscopy), dissolution properties, and mechanical testing. Additionally, a micromolding process was performed to reproduce negative microneedle molds made from PDMS, and as well fabrication of phantom gel tissue samples.

### PDMS Microneedle Mold Fabrication

In some example implementations, The fabrication of the PDMS negative MN molds was performed by casting a PDMS 8.6/1.4 (base/curing agent) solution (SYLGARD® 184) over a conical master MN mold made of acrylate resin (black-colored AnyCubic photon). Subsequently, PDMS was degassed for 15 minutes by placing the mold within a sealed desiccator connected to a vacuum pump running at 23 in Hg. Furthermore, the mold was left for 1 h at room temperature and later placed in an oven at 85° C. for 30 minutes. After the curing process, the negative mold was demolded from the master MN mold and resized with a blade cut. Prior use, each PDMS MN mold was cleaned/washed by triplicate with soap, ultrasonicated, temperature treated (80° C.), and stored in a sealed container.

### Fabrication of the Active MN Patch

In some example implementations, the active MN vaccination patches were fabricated by a micromolding technique with the use of negative PDMS MN molds. Briefly, 50 μL of a Mg microparticle (catalog #FMW40, from TangShanWeiHao Magnesium Powder Co., Ltd China) 2-propanol solution (50 mg/mL) was added to the negative MN mold to pack the cavities. Furthermore, a volume of 250 μL of a 10% w/v polyvinylpyrrolidone (PVP, MW=360 K, Sigma Aldrich) aqueous solution (pH 10.5 and pH 7.4) was casted over the negative molds in a closed desiccator at 23 in Hg for a total time of 10 minutes. Afterwards, bubbles were removed from the mold needle interface and repetitive additions of PVP solution were added to reach a total volume of 750 μL. The corresponding payload (100 μg of CPMV, Cy3-CPMV or Cy5-CPMV) was incorporated onto the mold and allowed to dry for 48 hours at room temperature in a sealed container. Upon drying, a circular 1.2 mm adhesive (3M scotch tape) was applied to the backing of the MN patches and demolded. Passive MNs were formulated by following identical preparation steps, however, the inclusion of Mg microparticles was not performed, respectively. Both active MN and passive MN patches were stored at room temperature for up to 2 weeks in a sealed container prior to use. For larger or more bulky tumors, MN patches were cut into 4 or 9 pieces to facilitate application.

### MN Patch Imaging Characterization and Dissolution Experiments

In some example implementations, the fluorescent microscopy images of the active MN platform were performed by the use of an EVOS FL microscope (2× and 4× objectives and RFP fluorescent filter) for the Cy3-CPMV imaging. Furthermore, the SEM images were obtained with the use of a FEI Quanta 250 ESEM instrument (Hillsboro, Oreg., USA). Samples were sputtered with Iridium (Emitech K575X Sputter Coater) to provide a fine grain metal deposition and imaged with acceleration voltages between 3-5 keV. For the dissolution experiments, arrays of only 3 conical active MNs were attached horizontally to a clear glass slide. To capture the dissolution in real time, PBS pH 6.5 was added to the MN array and images were taken with the use of an inverted optical microscope (Nikon Eclipse Instrument Inc. Ti-S/L100) coupled with a 4× microscope objective, a Hamamatsu digital camera C11440, and a NIS Elements AR 3.2 software.

### Release Kinetics Experiments

In some example implementations, after the MN patch fabrication, passive MN and active MN patches were used to pierce a phantom tissue. The synthetic phantom tissues were formulated with a 2% (w/v) Agarose (Sigma Aldrich) aqueous solution and further molded in custom made negative EcoFlex molds (1.5 mm diameter, 3 mm thickness). Phantom tissues were stored submerged in PBS (pH 6.5) and completely sealed prior to use. For testing, the passive MN and active MN patches loaded with Cy5-CPMV penetrated the phantom tissues for different durations: 1, 3, 5, 10, 20 and 30 minutes at 37.5° C. Following application, the patches were removed from the tissue and dissolved in 800 μL of PBS pH 6.5. The use of a UV-2450 Shimadzu spectrophotometer was used for the absorbance measurements from a 400-700 nm spectrum window and the release from patches was plotted vs time.

### Active MN Compression Test

In some example implementations, the mechanical compression test was performed by the use of a Force Gauge Model M4-20 system Mark0-10 Series 4. In brief, an active MN array was set under a constant load, and the displacement of the base plate in reference to each needle height was monitored and plotted. The fracture (failure) force was determined by a notorious drop in force.

### Cell Line

In some example implementations, the B16F10 cell line was acquired from American Type Culture Collection (ATCC). B16F10 cells were cultured in Dulbecco's Modified Eagle's Medium (DMEM, Life Technologies), supplemented with 10% (v/v) fetal bovine serum (FBS, Atlanta Biologicals) and 1% (v/v) penicillin-streptomycin (Life Technologies). Cells were maintained at 37° C., 5% C02. The cell cultures were maintained below a 50% confluence and early passage culture were utilized for the experiments.

### Expression and Purification of CPMV Nanoparticles

In some example implementations, CPMV was propagated in California Blackeye No. 5 cowpea plants and purified. Bioconjugation of Cy3 and Cy5 fluorophores to CPMV external lysine residues. The CPMV protein capsid consists of 180 coat proteins upon which 300 surface-exposed lysine side chains are displayed.61 CPMV nanoparticles were labeled with sulfo-Cy5-NHS (Abcam) using N-hydroxysuccinimide-activated esters that target the surface lysine residues. The reactions were carried out with a 1,200-fold CPMV molar excess of sulfo-Cy5-NHS in a 0.1 M KP buffer (pH 7.0) at room temperature overnight, with agitation. This yielded approximately 30 Cy5 fluorophores conjugated to each CPMV. For Cy3 the reactions were carried out with a 3,000-fold CPMV molar excess of sulfo-Cy3-NHS, which yielded approximately 50-70 Cy3 fluorophores per CPMV. Fluorophore-conjugated CPMV was characterized by UV-visual spectral analysis, transmission electron microscopy, gel (SDS-PAGE and 1.2% (w/v) agarose) analysis.

### In Vivo Efficacy Study in Mice

In some example implementations, experiments were conducted in accordance with UCSD's Institutional Animal Care and Use Committee. Six- to eight-week-old female C57BL/6 mice (The Jackson Laboratory) were used. For larger tumors, 250,000 B16F10 cells were suspended in 30 μL PBS and were injected intradermally into the right flank of each C57BL/6 mouse on day 0. PBS (30 μL) or CPMV (100 μg in 30 μL) were administered by IT injection into the base of the tumor or by MN patch on day 7. MNs were applied on the tumors for 5-10 minutes until the needles completely dissolved. A PBS solution of pH 5.1 was applied to the skin of the treated region, immediately following application of the active MN patch. For smaller tumors, 25,000 B16F10 cells were suspended in 50 μL PBS and were injected intradermally into the right flank of each C57BL/6 mouse on day 0. PBS (30 μL) or CPMV (100 μg in 30 μL) were administered into mice by IT injection or by microneedle on day 10. Tumor volumes were measured using a digital caliper. The tumor volume (mm³) was calculated as (long diameter×short diameter2)/2. Animals were sacrificed when tumor volume reached ≥1500 mm3.

### Tumor Immunofluorescence Imaging

In some example implementations, 250,000 B16F10 cells were injected intradermally into the left flank of each C57BL/6 mice on day 0 as described previously. When tumors reached a volume of 60-100 mm3, IT PBS (30 μL) injection, Cy5-CPMV (100 μg in 30 μL) injection, passive MN, or active MN patch Cy5-CPMV (100 μg) were administered. 24 h post treatment, tumors excised en bloc from flank with 2-3 mm margin of normal surrounding skin. Tissue was flash frozen in OCT media with isopentane (cooled by dry ice to −78.5° C.). Tumors were cryo-sectioned into 5 m transverse sections (orthogonal to the longest axis). Tumor sections were fixed with cooled 100% acetone (−20° C.), then washed with PBS and blocked (1×PBS/5% (v/v) normal goat serum (Cell Signaling Technology, 5425S)/0.3% (v/v) Triton X-100) for 1 h at room temperature. Primary antibody staining was subsequently performed overnight at 4° C. with rabbit anti-mouse CD31/PECAMI polyclonal antibody (Abcam, ab28364) at 1:50 dilution and rat anti-mouse CD45 (Cell Signaling Technology, clone 30-F11) at 1:800 dilution. Sections were then washed in PBS and stained with secondary antibodies (anti-rabbit Alexa Fluor 568 (Abcam, ab175471) at 1:1000 and anti-rat Alexa Fluor 488 (Cell Signaling Technology, 4416S) at 1:500) at room temperature for 2 h. After washing with PBS and drying, sections were counterstained and cover slipped with Prolong® Gold Antifade Reagent with DAPI (Cell Signaling Technology, 8961S). Sections were visualized on Keyence BZ-X710 all-in-one microscope (Keyence Corporation) with filter set (DAPI, TRITC, FITC, and Cy-5) and accompanying imaging analysis software.

### Tumor In Vivo Fluorescence Imaging

In some example implementations, the IVIS in vivo imaging system (IVIS Xenogen 200, Perkin Elmer) was used for non-invasive visualization and analysis of cutaneous distribution and retention of Cy5-CPMV administered via IT conventional injection, passive MN, and active MN in vivo. Mice were fed an alfalfa-free diet (PicoLab High Energy Mouse Diet, 5LJ5) starting at least lweek prior to imaging. 250,000 B16F10 cells were injected intradermally into the left flank of each C57BL/6 mouse on day 0 as described previously. When tumors reached a volume of 60-100 mm3, IT PBS injection, Cy5-CPMV injection, passive MN, or active MN patch Cy5-CPMV were administered, with doses as described previously. Mice were imaged with the Cy5.5 filter (excitation range 615-665 nm and emission range 695-770 nm, 0.5 s exposure) under anesthesia, before treatment (baseline or ‘BL’), and after treatment at specified timepoints (0 h, 4 h, 8 h, 12 h, 24 h, 36 h, 48 h, 72 h, and 96 h). Living Image Software (version 4.3.1, Perkin Elmer) was used to analyze all fluorescence data in this study. A region of interest (ROI) was drawn around each tumor and the measured fluorescence (in radiant efficiency, (p/sec/cm²/sr)/(μW/cm²)) was calculated and normalized to the baseline ROI fluorescence for each tumor.

### Flow Cytometry

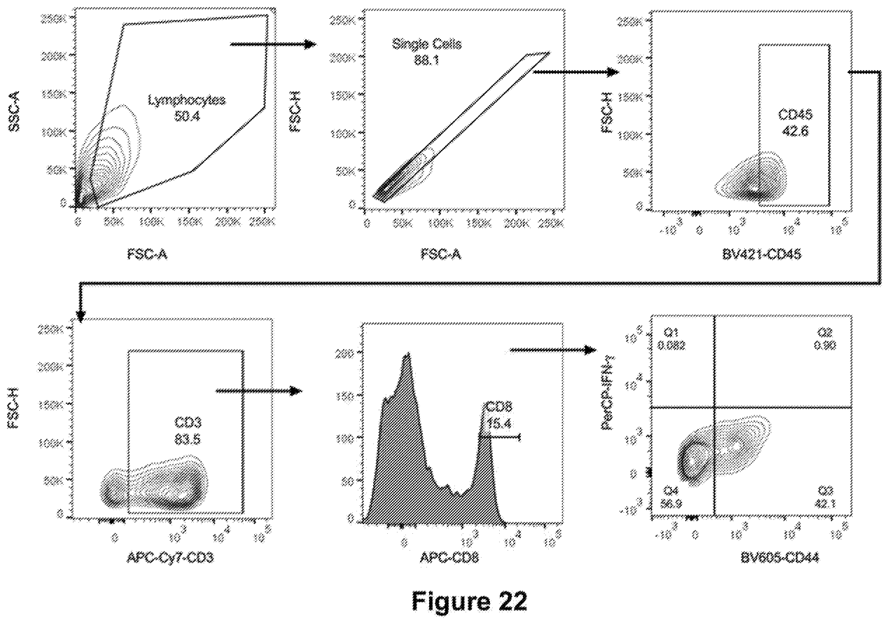

In some example implementations, for tumor immunoprofiling and splenocyte interferon gamma (IFN-γ) release assays, fresh, single-cell suspensions were made from excised B16F10 melanomas and spleens, respectively. Cells were washed in cold PBS containing 1 mM EDTA, and then resuspended in staining buffer (PBS containing 2% (v/v) FBS, 1 mM EDTA, 0.1% (w/v) sodium azide). Fc receptors were blocked using anti-mouse CD16/CD32 (BioLegend) for 15 min and then tested with the following fluorescence-labeled antibodies (BioLegend) for 30 min at 4° C.: CD45 (30-F11), CD11b (M1/70), CD86 (GL-1), major histocompatibility complex class II (MHCII, M5/114.15.2), Ly6G (1A8), CD11c (N418 A), F4/80 (BM8), Ly6C (HK1.4), NK1.1 (PK136), CD4 (GK1.5), CD3ε (145-2V11 A), CD8α (53-6.7), CD44 (IM7), CD62L (MEL-14), and isotype controls. The gating strategy used for tumor immunoprofiling analyses were as previously described. The gating strategy used for splenocyte IFN-γ release assays are presented in FIG. 22. For intracellular cytokine staining, splenocytes (106 cells/mL) were co-cultured with freeze-thawed B16F10 melanoma cell lysate (106 cells/mL) or CPMV (0.1 mg/mL) for 48 h and treated with brefeldin A (10 mg/mL) for the last 5 h at 37° C. Following staining for surface antibodies as described above, the cells were fixed in 3% (w/v) paraformaldehyde, permeabilized with 0.1% (w/v) saponin, then incubated with anti-IFN-γ (XMG1.2, BioLegend) for 30 min in 0.1% (w/v) saponin. Cells were washed twice and resuspended in staining buffer for data acquisition. Flow cytometry was carried out using a BD LSRII cytometer (BD Biosciences), and the data were analyzed using FlowJo software (Tree Star). OneComp eBeads (eBiosciences) were used as compensation controls.

In conclusion, disclosed is an effective microneedle delivery route that offers active payload delivery, without the use of external stimuli, towards improved outcome vs commonly used passive diffusive microneedle transport. Such active degradable and autonomous microneedle delivery has been realized through the incorporation of Mg microparticles loaded within the microneedle patch, that produce a built-in pump source for faster and deeper intradermal local delivery. Such autonomous pumping action obviates the need for expensive and bulky external device essential for generating external stimuli. The presently disclosed technology allows the fabrication of a microneedle patch as well for combinatorial delivery using spatially-resolved active and passive microneedle zones, for fast/deep and slow sustained release, respectively. Moreover, the new active microneedle delivery system is not limited to specific polymeric materials or microneedle geometry or dimensions. The new “built-in” active delivery strategy holds considerable promise for diverse practical biomedical applications for transdermal delivery, including drug delivery, immunotherapy, along with cosmetic treatment, offering an attractive alternative to patients in the clinic when compared to traditional over the counter medical devices. In addition, the new active/passive combination delivery patch offers tremendous promise for pain killing and cardiac treatment applications towards diverse future biomedical applications in centralized and decentralized settings.

The presently disclosed technology has tremendous commercial promises as it could be implemented for diverse practical biomedical applications for transdermal drug delivery, immunotherapy, pain killing, and more. More specifically, this technology can lead to translation to the clinic due to its fully biocompatibility and autonomous nature.

### EXAMPLES

In some embodiments in accordance with the present technology (example A1), an autonomous cargo delivery device comprising a microneedle patch that comprises one or more microneedles loaded with a therapeutic payload and an active microparticle.

Example A2 includes the device of any of examples A1-A5, wherein the one or more microneedles are degradable.

Example A3 includes the device of any of examples A1-A5, wherein the active microparticle reacts with subcutaneous biofluid upon insertion of the microneedle patch to enhance transport of the therapeutic payload.

Example A4 includes the device of any of examples A1-A5, wherein the active microparticle includes magnesium particles.

Example A5 includes the device of any of examples A1-A4, wherein the microneedle patch further comprises one or more passive microneedles.

In some embodiments in accordance with the present technology (example A6), a method of autonomous cargo delivery into a subject, comprising using a microneedle patch that comprises one or more microneedles loaded with a therapeutic payload and an active microparticle.

Example A7 includes the method of any of examples A6-A10, wherein the one or more microneedles are degradable.

Example A8 includes the method of any of examples A6-A10, wherein the active microparticle reacts with subcutaneous biofluid upon insertion of the microneedle patch to enhance transport of the therapeutic payload.

Example A9 includes the method of any of examples A6-A10, wherein the active microparticle is magnesium particles.

Example A10 includes the method of any of examples A6-A9, wherein the microneedle patch further comprises one or more passive microneedles.

In some embodiments in accordance with the present technology (example A11), a device for autonomous delivery of a molecular payload in tissue includes a substrate; and an array of microneedles on the substrate, the microneedles including a degradable polymeric microneedle body loaded with the molecular payload and a plurality of particles configured to react with surrounding subcutaneous biofluid when the microneedles are inserted in the tissue.

Example A12 includes the device of any of examples A11-A20, wherein the plurality of particles include magnesium microparticles capable to react with the surrounding subcutaneous biofluid to generate hydrogen bubbles that induce a vortex flow field to cause a dynamic transport of the molecular payload in the tissue.

Example A13 includes the device of any of examples A11-A20, wherein the dynamic transport of the molecular payload includes a pump-like action between the microneedles and the tissue.

Example A14 includes the device of any of examples A11-A20, wherein the plurality of particles includes a biocatalytic enzyme, an inorganic material, or a composite material configured to convert a local chemical fuel in the tissue or an external field applied at the tissue into leading forces that induce fluid transport.

Example A15 includes the device of any of examples A11-A20, wherein the biocatalytic enzyme includes glucose oxidase.

Example A16 includes the device of any of examples A11-A20, wherein the inorganic material includes a metal catalyst.

Example A17 includes the device of any of examples A11-A20, wherein the composite material includes a metal-organic framework composite.

Example A18 includes the device of any of examples A11-A20, wherein the external field includes a magnetic field, an ultrasonic field, or an optical field.

Example A19 includes the device of any of examples A11-A20, wherein the substrate includes a flexible substrate able to attach and conform to skin tissue.

Example A20 includes the device of any of examples A11-A19, wherein the device is configured to implement the method of any of examples A6-A10.

In some embodiments in accordance with the present technology (example B1), a microneedle therapeutic payload delivery device includes a substrate; an activation particle; and one or more degradable microneedle structures coupled to the substrate and including a polymeric matrix structured to embed the activation microparticle and one or more therapeutic payloads, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of a biofluid surrounding the microneedle structure to dissolve and allow the one or more therapeutic payloads and the activation particle to the surrounding biofluid.

Example B2 includes the device of any of examples B1-B15, wherein the activation particle is configured to react with biofluid to enhance transport of the one or more therapeutic payloads into a tissue by dispersion of the one or more therapeutic payloads away from the one or more degradable microneedle structures and penetration of the one or more therapeutic payloads deeper into the tissue.

Example B3 includes the device of any of examples B1-B15, wherein the activation particle is microparticle or nanoparticle.

Example B4 includes the device of any of examples B1-B15, wherein the activation particle includes magnesium microparticles.

Example B5 includes the device of any of examples B1-B15, wherein the activation particle is coated with an enteric polymer.

Example B6 includes the device of any of examples B1-B15, wherein the activation particle is a chemically modified microparticle or nanoparticle with at least one of biocatalytic enzymes, an inorganic material, or a microparticle or nanoparticle modified metal organic frameworks (MOF).

Example B7 includes the device of any of examples B1-B15, wherein the polymeric matrix is formed of a transient degradable material including one or more of polyvinylpyrrolidone (PVP), carboxymethylcellulose (CMC), hyaluronic acid (HA), sodium alginate (SA), or Pullulan.

Example B8 includes the device of any of examples B1-B15, further comprising one or more passive microneedle structures coupled to the substrate and including a nondegradable material, the one or more passive microneedle structures each including an external wall spanning outward from a base surface and forming an apex at a terminus point of the external wall.

Example B9 includes the device of any of examples B1-B15, wherein: the one or more therapeutic payloads includes a therapeutic agent selected from the group consisting of immune oncology agents, chemotherapeutic agents, chronic pain agents, cardiovascular agents, anti-aging agents, antiviral agents, vaccines, antibacterial agents, micronutrients, and gene editing effectors, and/or wherein the one or more therapeutic payloads includes a nanoparticle to which the therapeutic agent is attached; and/or the one or more therapeutic payloads includes a one or more of a drug, particle, molecule, genetic material, protein, virus-like particle, virus, enzyme, nanoparticle, or combination thereof.

Example B10 includes the device of any of examples B1-B15, wherein the one or more therapeutic payloads includes a first therapeutic agent embedded within a first microneedle structure of the one or more degradable microneedle structures, and a second therapeutic agent embedded within a second microneedle structure of the one or more degradable microneedle structures.

Example B11 includes the device of any of examples B1-B15, wherein the first therapeutic agent is releasable into the biofluid surrounding the microneedle structure before a release of the second therapeutic agent.

Example B12 includes the device of any of examples B1-B15, wherein the one or more therapeutic payloads includes a first therapeutic agent and a second embedded within at least one microneedle structure of the one or more degradable microneedle structures.

Example B13 includes the device of any of examples B1-B15, wherein the substrate includes an adhesive material on at least a side of the substrate interfaced with the one or more degradable microneedle structures.

Example B14 includes the device of any of examples B1-B15, wherein the environmental parameter includes a pH of less than 7.0.

Example B15 includes the device of any of examples B1-B14, wherein the biofluid includes interstitial fluid (ISF).

In some embodiments in accordance with the present technology (example B16), a method for autonomously delivering a payload into a biofluid via microneedles includes providing a microneedle patch device that includes a substrate and one or more degradable microneedle structures coupled to the substrate and including a polymeric matrix structured to embed a activation microparticle and one or more payload substances, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of the biofluid that would surround the microneedle structure; applying the microneedle patch device to skin of a subject such that the one or more degradable microneedle structures are inserted into a tissue; dissolving the one or more degradable microneedles in the fluid based on exposure of the one or more degradable microneedles to the environmental parameter; and reacting the activation particle to the biofluid to cause the one or more payload substances to disperse away from the one or more degradable microneedle structures, thereby enhancing penetration of the one or more payload substances into the tissue.

Example B17 includes the method of any of examples B16-B27, wherein the activation particle includes magnesium, and wherein the reacting includes: oxidizing the magnesium of the activation particle from Mg⁰ to Mg⁺² after reacting with H⁺ ions in the biofluid, resulting in production of gaseous H₂, and generating a vortex within the biofluid by the gaseous H₂, thereby mixing the one or more payload substances in the biofluid and driving the one or more payload substances deeper into the tissue.

Example B18 includes the method of any of examples B16-B27, wherein the activation particle includes an enteric coating.

Example B19 includes the method of any of examples B16-B27, wherein the activation particle is microparticle or nanoparticle.

Example B20 includes the method of any of examples B16-B27, wherein the activation particle is a chemically modified microparticle or nanoparticle with at least one of biocatalytic enzymes, an inorganic material, or a microparticle or nanoparticle modified metal organic frameworks (MOF).

Example B21 includes the method of any of examples B16-B27, wherein the polymeric matrix is formed of a transient degradable material including one or more of polyvinylpyrrolidone (PVP), carboxymethylcellulose (CMC), hyaluronic acid (HA), sodium alginate (SA), or Pullulan.

Example B22 includes the method of any of examples B16-B27, wherein the tissue is a tumor, and the applying the microneedle patch device to the skin includes inserting the one or more degradable microneedle structures into the tissue without inserting the one or more degradable microneedle structures into one or both of blood vessels or underlaying subcutaneous tissue.

Example B23 includes the method of any of examples B16-B27, wherein the one or more therapeutic loads is an immune oncology agent and the enhanced transport of the one or more therapeutic loads induces an early cellular innate immune response in the biofluid.

Example B24 includes the method of any of examples B16-B27, wherein the tissue includes subcutaneous tissue.

Example B25 includes the method of any of examples B16-B27, wherein the one or more payload substances includes a therapeutic agent selected from the group consisting of immune oncology agents, chemotherapeutic agents, chronic pain agents, cardiovascular agents, anti-aging agents, antiviral agents, vaccines, antibacterial agents, micronutrients and gene editing effectors; and/or the one or more payload substances includes a one or more of a drug, particle, molecule, genetic material, protein, virus-like particle, virus, enzyme, nanoparticle, or combination thereof.

Example B26 includes the method of any of examples B16-B27, further comprising prior to the providing, attaching a therapeutic agent to a nanoparticle; and loading the nanoparticle with attached therapeutic agent into the polymeric matrix of the microneedle structure.

Example B27 includes the method of any of examples B16-B26, wherein the one or more payload substances includes a cowpea mosaic virus nanoparticle complex, the method comprising: applying the microneedle patch device to a region of the skin having a melanoma, such that the one or more degradable microneedle structures are inserted into the melanoma, wherein the reacting the activation particle cause the cowpea mosaic virus nanoparticle complex to disperse away from the one or more degradable microneedle structures via a propulsive force generated by the reacting to drive the cowpea mosaic virus nanoparticle complex into the melanoma to cause restructuring of a tumor microenvironment of the melanoma.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described, and other implementations, enhancements and variations can be made based on what is described and illustrated in this patent document.

1. A microneedle therapeutic payload delivery device, comprising:
a substrate;
an activation particle; and
one or more degradable microneedle structures coupled to the substrate and including a polymeric matrix structured to embed the activation microparticle and one or more therapeutic payloads, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of a biofluid surrounding the microneedle structure to dissolve and allow the one or more therapeutic payloads and the activation particle to the surrounding biofluid.

2. The device of claim 1, wherein the activation particle is configured to react with biofluid to enhance transport of the one or more therapeutic payloads into a tissue by dispersion of the one or more therapeutic payloads away from the one or more degradable microneedle structures and penetration of the one or more therapeutic payloads deeper into the tissue.

3. The device of claim 1, wherein the activation particle is microparticle or nanoparticle, and wherein the activation particle is coated with an enteric polymer.

4. The device of claim 1, wherein the activation particle includes magnesium microparticles.

5. The device of claim 1, wherein the activation particle is coated with an enteric polymer.

6. The device of claim 1, wherein the activation particle is a chemically modified microparticle or nanoparticle with at least one of biocatalytic enzymes, an inorganic material, or a microparticle or nanoparticle modified metal organic frameworks (MOF).

7. The device of claim 1, wherein the polymeric matrix is formed of a transient degradable material including one or more of polyvinylpyrrolidone (PVP), carboxymethylcellulose (CMC), hyaluronic acid (HA), sodium alginate (SA), or Pullulan.

8. The device of claim 1, further comprising:
one or more passive microneedle structures coupled to the substrate and including a nondegradable material, the one or more passive microneedle structures each including an external wall spanning outward from a base surface and forming an apex at a terminus point of the external wall.

9. The device of claim 1, wherein the one or more therapeutic payloads includes a therapeutic agent selected from the group consisting of immune oncology agents, chemotherapeutic agents, chronic pain agents, cardiovascular agents, anti-aging agents, antiviral agents, vaccines, antibacterial agents, micronutrients, and gene editing effectors, and/or wherein the one or more therapeutic payloads includes a nanoparticle to which the therapeutic agent is attached.

10. The device of claim 1, wherein the one or more therapeutic payloads includes one or more of a drug, particle, molecule, genetic material, protein, virus-like particle, virus, enzyme, nanoparticle, or combination thereof.

11. The device of claim 1, wherein the one or more therapeutic payloads includes a first therapeutic agent embedded within a first microneedle structure of the one or more degradable microneedle structures, and a second therapeutic agent embedded within a second microneedle structure of the one or more degradable microneedle structures.

12. The device of claim 11, wherein the first therapeutic agent is releasable into the biofluid surrounding the microneedle structure before a release of the second therapeutic agent.

13. The device of claim 1, wherein the one or more therapeutic payloads includes a first therapeutic agent and a second embedded within at least one microneedle structure of the one or more degradable microneedle structures.

14. The device of claim 1, wherein the substrate includes an adhesive material on at least a side of the substrate interfaced with the one or more degradable microneedle structures.

15. The device of claim 1, wherein the environmental parameter includes a pH of less than 7.0.

16. (canceled)

17. A method for autonomously delivering a payload into a biofluid via microneedles, the method comprising:
providing a microneedle patch device that includes a substrate and one or more degradable microneedle structures coupled to the substrate and including a polymeric matrix structured to embed a activation microparticle and one or more payload substances, the one or more degradable microneedle structures each including an exterior wall spanning outward from a base surface and forming an apex at a terminus point of the exterior wall, wherein the polymeric matrix of a microneedle structure of the one or more degradable microneedle structures is degradable under an environmental parameter of the biofluid that would surround the microneedle structure;
applying the microneedle patch device to skin of a subject such that the one or more degradable microneedle structures are inserted into a tissue;
dissolving the one or more degradable microneedles in the fluid based on exposure of the one or more degradable microneedles to the environmental parameter; and
reacting the activation particle to the biofluid to cause the one or more payload substances to disperse away from the one or more degradable microneedle structures, thereby enhancing penetration of the one or more payload substances into the tissue.

18. The method of claim 17, wherein the activation particle includes magnesium, and wherein the reacting includes:
oxidizing the magnesium of the activation particle from Mg⁰ to Mg⁺² after reacting with H⁺ ions in the biofluid, resulting in production of gaseous H₂, and generating a vortex within the biofluid by the gaseous H₂, thereby mixing the one or more payload substances in the biofluid and driving the one or more payload substances deeper into the tissue.

19. (canceled)

20. (canceled)

21. (canceled)

22. (canceled)

23. The method of claim 17, wherein the tissue is a tumor, and the applying the microneedle patch device to the skin includes inserting the one or more degradable microneedle structures into the tissue without inserting the one or more degradable microneedle structures into one or both of blood vessels or underlaying subcutaneous tissue.

24. (canceled)

25. (canceled)

26. (canceled)

27. (canceled)

28. The method of claim 17, further comprising:
prior to the providing, attaching a therapeutic agent to a nanoparticle; and
loading the nanoparticle with attached therapeutic agent into the polymeric matrix of the microneedle structure.

29. The method of claim 28, wherein the one or more payload substances includes a cowpea mosaic virus nanoparticle complex, the method comprising:
applying the microneedle patch device to a region of the skin having a melanoma, such that the one or more degradable microneedle structures are inserted into the melanoma, wherein the reacting the activation particle cause the cowpea mosaic virus nanoparticle complex to disperse away from the one or more degradable microneedle structures via a propulsive force generated by the reacting to drive the cowpea mosaic virus nanoparticle complex into the melanoma to cause restructuring of a tumor microenvironment of the melanoma.

30. A device for autonomous delivery of a molecular payload in tissue, comprising:
a substrate; and
an array of microneedles on the substrate, the microneedles including a degradable polymeric microneedle body loaded with the molecular payload and a plurality of activation particles configured to react with surrounding biofluid when the microneedles of the array are inserted in a tissue.

31. The device of claim 30, wherein the plurality of activation particles includes magnesium microparticles capable to react with the surrounding subcutaneous biofluid to generate hydrogen bubbles that induce a vortex flow field to cause a dynamic transport of the molecular payload in the tissue.

32. The device of claim 30, wherein the dynamic transport of the molecular payload includes a pump-like action between the microneedles and the tissue.

33. The device of claim 30, wherein the plurality of activation particles includes a biocatalytic enzyme, an inorganic material, or a composite material configured to convert a local chemical fuel in the tissue, or an external field applied at the tissue into leading forces that induce fluid transport.

34. The device of claim 33, wherein:
the biocatalytic enzyme includes glucose oxidase;
the inorganic material includes a metal catalyst;
the composite material includes a metal-organic framework composite; and/or
the external field includes a magnetic field, an ultrasonic field, or an optical field.

35. The device of claim 30, wherein the substrate includes a flexible substrate able to attach and conform to skin tissue.
